(19) United States
(12) Patent Application Publication
(10) Pub. No.: US 2026/0053812 A1
(43) Pub. Date: Feb. 26, 2026

(54) METHODS OF TREATING HYPERTENSION WITH A COMBINATION OF AN ALDOSTERONE SYNTHASE INHIBITOR AND A DIURETIC

(71) Applicant: Mineralys Therapeutics, Inc., Radnor, PA (US)

(72) Inventors: David Rodman, Radnor, PA (US); Jon Congleton, Radnor, PA (US)

(73) Assignee: Mineralys Therapeutics, Inc., Radnor, PA (US)

(21) Appl. No.: 19/104,845

(22) PCT Filed: Aug. 22, 2023
(86) PCT No.: PCT/US2023/072661

§ 371 Date: Feb. 19, 2025

Related U.S. Application Data
(60) Provisional application No. 63/400,312, filed on Aug. 23, 2022.
(60) Provisional application No. 63/425,803, filed on Nov. 16, 2022.

(51) Int. Cl. A61K 31/53; A61K 31/549; A61P 9/12; G01N 33/74

(52) U.S. Cl. CPC A61K 31/53; A61K 31/549; A61P 9/12; G01N 33/74; G01N 33/743; G01N 2800/321; G01N 2800/52

(57) ABSTRACT

This invention provides a method of treating hypertension in a hypertensive subject, the method administering to the subject, an amount of a diuretic and an amount of a CYP 11β2 beta hydroxylase inhibitor once or twice per day, wherein the amounts taken together are sufficient to treat hypertension in the hypertensive subject when taken in combination with other antihypertensive agents. In particular, the method is sufficient to elicit a safe and robust reduction in hypertension (>10 mmHg reduction in systolic blood pressure) in the hypertensive subject.

This application claims priority of U.S. Provisional Application No. 63/425,803, filed Nov. 16, 2022 and U.S. Provisional Application No. 63/400,312, filed Aug. 23, 2022 the contents of each of which are hereby incorporated by reference in their entirety.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### FIELD OF THE INVENTION

This invention relates to methods of treating hypertension by inhibiting aldosterone synthase (CYP 11β2 beta hydroxylase).

### BACKGROUND OF THE INVENTION

Aldosterone is the principal mineralocorticoid in humans, produced in the zona glomerulosa of the adrenal cortex by aldosterone synthase (CYP 11β2 beta hydroxylase). Aldosterone is a key component of the renin-angiotensin-aldosterone system (RAAS), acting primarily as a regulator of electrolyte and fluid homeostasis.

Mineralocorticoid receptor-blocking agents (mineralocorticoid receptor antagonists, MRA), such as spironolactone and eplerenone, prevent aldosterone from binding with the mineralocorticoid receptor. Several clinical studies have demonstrated their benefit in treating hypertension. Given the role aldosterone plays in RAAS, inhibition of aldosterone synthase represents a possible alternative to mineralocorticoid receptor-blocking agents for the treatment of hypertension. However, previous studies suggest that some of the effects of aldosterone may occur independently of stimulation of the mineralocorticoid receptor/classic steroid-receptor complex modulation (Grossmann, C., & Gekle, M., 2009; Good, D. W., 2007; Mihailidou, A. S., & Funder, J. W., 2005). In addition, mineralocorticoid receptors are not selective for aldosterone, instead having similar affinity for the glucocorticoids cortisol and corticosterone.

Diuretics are medications designed to increase the amount of water and salt expelled from the body as urine. Thiazide diuretics are the most commonly prescribed diuretic, often used to treat hypertension. Thiazide diuretics not only decrease fluids, but also cause blood vessels to relax. Thiazide diuretics reduce potassium concentration in blood. This reduction in potassium occurs through two indirect mechanisms: (1) inhibition of sodium-chloride symporter at distal convoluted tubule of a nephron and (2) stimulation of aldosterone which activates Na+/K+-ATPase at the collecting duct. Sodium-chloride symporter inhibition increases the availability of chloride and sodium in urine. When urine reaches the collecting duct, the increase in chloride and sodium availability activates Na+/K+-ATPase, which in turn increases absorption of sodium and excretion of potassium into urine. Long term administration of thiazide diuretics reduces total body blood volume. This activates the renin-angiotensin system, stimulating the secretion of aldosterone, thus activating Na+/K+-ATPase and increasing potassium excretion in urine. Therefore, a combination of ACE inhibitors and thiazide is used to prevent hypokalemia.

The administration of two drugs to treat a given condition, such as the combination of an aldosterone synthase inhibitor and a thiazide to treat hypertension, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Food and Drug Administration, 2020). Thus, when two drugs are administered to treat the same condition, it is unpredictable whether each will complement, have no effect on, or interfere with, the therapeutic activity of the other in a human subject. Interactions between two drugs may also heighten or lessen the side effects of each drug. Hence, upon administration of two drugs to treat a disease, it is unpredictable what change will occur in the negative side profile of each drug.

The renin-angiotensin-aldosterone system (RAAS) is a complex and multi-faceted system, with many different pathways, enzymes, and hormones interacting in positive and negative feedback loops. Given the complexities of the renin-angiotensin-aldosterone system, use of aldosterone synthase inhibitors to treat hypertension in combination with other anti-hypertensive therapies carries many uncertainties. In particular, the effect of a combination of an aldosterone synthase inhibitor and a diuretic such as thiazide on hypertension has not been previously studied. Further, identification of specific patient populations who benefit most from inhibition of aldosterone synthase remains an important goal in the field of cardiovascular health.

### SUMMARY OF THE INVENTION

This invention provides a method of treating hypertension in a hypertensive subject, the method administering to the subject, an amount of a diuretic and an amount of a CYP 11β2 beta hydroxylase inhibitor once or twice per day, wherein the amounts taken together are sufficient to treat hypertension in the hypertensive subject, preferably when taken in combination with other antihypertensive agents. In particular, the method is sufficient to elicit a safe and robust reduction in hypertension (>10 mmHg reduction in systolic blood pressure) in the hypertensive subject.

This invention also provides a method of treating hypertension in a hypertensive subject who is taking at least one diuretic, the method comprising administering to the subject a CYP 11β2 beta hydroxylase inhibitor once or twice per day in an amount sufficient to thereby treat hypertension in the hypertensive subject.

This invention also provides a method of identifying a subject for hypertension treatment with a CYP 11β2 beta hydroxylase inhibitor, the method comprising:
(a) measuring:
   (i) a systolic blood pressure of greater than 130 mmHg in said subject; and
   (ii) a diastolic BP of greater than 80 mmHg in said subject; and
(b) selecting a subject who is taking at least one diuretic;
thereby identifying said subject for hypertension treatment with a CYP 11β2 beta hydroxylase inhibitor.

This invention also provides pharmaceutical compositions, packages, and unit dosage forms for use in any one of the methods described herein.

### BRIEF DESCRIPTION OF THE DRAWINGS

FIG. 1: Compound A HBr Study Schema. ABPM=ambulatory blood pressure monitoring; BP=blood pressure; BID=twice daily; EOT=end of treatment; FU=follow up; PRA=plasma renin activity; QD=once daily. ^{a}=If Screening results were available, inclusion/exclusion evaluation was performed. If subject was not eligible based on Screening results, they did not continue to Visit 4. If Screening results were not available, subject proceeded to Visit 4. If Screening results were not available at Visit 4, subject should attend Visit 5 to determine final eligibility. If eligible based on Screening results, ABPM assessment begins at Visit 5. ^{b}=The ABPM procedure initiated at home approximately 24 hours before Randomization (Study Day 1). Alternatively, sites were permitted choose to schedule an office visit on Study Day 0 (Visit 5) to initiate the ABPM procedure. Training for the ABPM procedure was done either at an office visit or via phone.

FIG. 2: Waterfall plots showing the AOBP change in systolic blood pressure at week 8. The figure shows waterfall plots of the full analysis and safety set (FAS) analysis of placebo, 50 mg QD and 100 mg QD groups and the per-protocol (PP) analysis of the 100 mg group. A modeled mean and per-protocol observed mean values are also shown for each group.

FIG. 3: Waterfall plots showing the AOBP change in systolic blood pressure at week 8. The figure shows waterfall plots of the FAS analysis of 12.5 mg QD, 12.5 mg BID, and 25 mg BID using all subjects with week 8 measurement. Modeled mean and per-protocol observed mean values are also shown for each group.

FIG. 4: A bar graph showing the mean change in systolic blood pressure from baseline. The figure provides a final analysis including both full analysis set (FAS, all evaluable subjects receiving at least one dose of Compound A HBr) and per-protocol (PP, only those receiving ≥75% of study drug with week 8 visit). Part 2 data shows the interim average of last visit week 5-6.

FIG. 5: Graph of mean observed automated office blood-pressure change from baseline at week 8 for QD dosing regimens, showing a dose-response to Compound A HBr. BID per-protocol cohorts are shown on the far right of the graph.

FIG. 6: Graph showing the systolic blood pressure change from baseline at week 8 for a 50 mg QD, 100 mg QD, 12.5 mg BID, and 25 mg BID pooled cohort, the lowest response quartile of the pooled cohorts, the highest response quartile of the pooled cohort, and placebo.

FIG. 7: A waterfall plot showing change in systolic blood pressure from placebo and 100 mg QD groups pooled from both Parts 1 and 2. Part 2 data from interim snapshot with all subjects randomized and average last visit week 5-6, minimum week 2.

FIG. 8: A graph showing the change in estimated glomerular filtration rate (eGFR) in different dosing cohorts.

FIG. 9: A graph showing an example of ambulatory 24-hour blood pressure monitoring. The graph shows the 24-hour ambulatory blood pressure (systolic) of a subject receiving Compound A HBr 100 mg QD versus baseline, showing an average 24-hour blood pressure reduction and restoration of normal nocturnal dipping pattern.

FIG. 10: A graph showing the change in systolic blood pressure at week 8 relative to baseline as measured using the ABPM full analysis set.

FIG. 11: Waterfall plots showing the 24-hour average and overnight average ABPM change at 8 weeks relative to baseline. 100 mg QD dose levels provide excellent 24-hour blood pressure reduction. Overnight blood pressure reduction from the 100 mg QD dose level appears to be superior to 25 mg BID.

FIG. 12: Modelled (MMRM) Least Square Mean (SEM) Change from Baseline at Week 8 in Seated Automated Office-Measured Systolic Blood Pressure (mmHg), Part 1 (Full Analysis Set). Error bars represent SEM. Numbers inside the bars represent the number of subjects with non-missing data. P-values represent statistically significant difference compared with placebo. *p=0.0114. **p=0.042. Abbreviations: BID, twice daily; mg, milligrams; mmHg, millimeters of mercury; QD, once daily; SBP, systolic blood pressure; SEM, standard error of the mean.

FIG. 13: Modelled (MMRM) Least Square Mean (SEM) Change from Baseline at Week 8 in Seated Automated Office-Measured Systolic Blood Pressure (mmHg), Parts 1 & 2 (Full Analysis Set). Error bars represent SEM. Numbers inside the bars represent the number of subjects with non-missing data. Abbreviations: mg, milligrams; mmHg, millimeters of mercury; MMRM, mixed model repeated measures; QD, once daily; SBP, systolic blood pressure; SEM, standard error of the mean.

FIG. 14: Boxplot of Measured Change from Baseline at Week 8 in Seated Automated Office-Measured Systolic Blood Pressure (mmHg), Part 1 (Full Analysis Set). ⋄, mean; —, median; • outlier. Upper and lower boundaries of each box represent 1st and 3rd quartiles. Abbreviations: BID, twice daily; mg, milligrams; mmHg, millimeters of mercury; QD, once daily; SBP, systolic blood pressure.

FIG. 15: Boxplot of Measured Change from Baseline at Week 8 in Seated Automated Office-measured Systolic Blood Pressure (mmHg) in Subjects Randomized to 100 mg QD, Parts 1 & 2 (Full Analysis Set). ⋄, mean; —, median; 1, • outlier. Upper and lower boundaries of each box represent 1st and 3rd quartiles. Abbreviations: mg, milligrams; mmHg, millimeters of mercury; QD, once daily; SBP, systolic blood pressure.

FIG. 16: Proportion of Subjects with Seated Automated Office-Measured SBP/DBP ≤130/80 mmHg at Week 8, Part 1 (Full Analysis Set). Subjects missing an assessment at Week 8 or who had received rescue medications prior to Week 8 were considered as failures. Numbers inside bars represent the number of subjects achieving AOBP ≤130/80 mmHg.

FIG. 17: Proportion of Subjects with Seated Automated Office-Measured SBP/DBP ≤130/80 mmHg at Study Week 8 in Subjects Randomized to 100 mg QD, Parts 1 & 2 (Full Analysis Set). Subjects missing an assessment at Week 8 or who had received rescue medications prior to Week 8 were considered as failures. Numbers inside bars represent the number of subjects achieving AOBP ≤130/80 mmHg.

FIG. 18: Time (Weeks) to First Occurrence of Seated Automated Office-Measured SBP/DBP ≤130/80 mmHg, Part 1 (Full Analysis Set). Subjects who failed to achieve a BP of ≤130/80 mmHg prior to Week 8/EoT were censored at the Week 8/EoT date. Subjects who were lost to follow-up prior to Week 8/EoT were censored at the date of the last known BP assessment. Subjects who took rescue medications were censored on the date of rescue medication initiation. Missing events correspond to subjects who didn't have post-baseline assessments for BP. For subject 146-040, the study duration (>27 weeks) and treatment duration (>18 weeks) were longer than planned due to AEs leading to temporary discontinuation.

FIG. 19: Time (Weeks) to First Occurrence of Seated Automated Office-Measured SBP/DBP ≤130/80 mmHg in Subjects Randomized to 100 mg QD, Parts 1 & 2 (Full Analysis Set). Subjects who failed to achieve a BP of ≤130/80 mmHg prior to Week 8/EoT were censored at the Week 8/EoT date. Subjects who were lost to follow-up prior to Week 8/EoT were censored at the date of the last known BP assessment. Subjects who took rescue medications were censored on the date of rescue medication initiation. Missing events correspond to subjects who didn't have post-baseline assessments for BP.

FIG. 20: Mean (SEM) Change in Serum Potassium from Baseline over Time in QD Dose Cohorts, Part 1 (Safety Analysis Set).

FIG. 21: Mean (SEM) Change in Serum Potassium from Baseline over Time in BID Dose Cohorts, Part 1 (Safety Analysis Set).

FIG. 22: Mean (SEM) Change in Serum Potassium from Baseline over Time, Part 2 (Safety Analysis Set).

FIG. 23: Mean (SEM) Change in Serum Sodium from Baseline over Time in QD Dose Cohorts, Part 1 (Safety Analysis Set)

FIG. 24: Mean (SEM) Change in Serum Sodium from Baseline over Time in BID Dose Cohorts, Part 1 (Safety Analysis Set)

FIG. 25: Mean (SEM) Change in Serum Sodium from Baseline over Time, Part 2 (Safety Analysis Set)

FIG. 26: Relationship between median baseline body mass index (BMI, kg/m2) and serum leptin (ng/dl). Data was derived from pooled data from low renin subjects (Part-1) from the lorundrostat 25 mg BID, 50 mg QD and 100 mg QD cohorts. Each of these cohorts demonstrated significant reduction in median serum aldosterone at week 4 compared to baseline (9.6%, 65.2%, 70.0%, respectively).

FIG. 27: Relationship between BMI at baseline and mean change in systolic BP (mmHg), measured by AOBP, at week 8 compared to baseline. Data was derived from pooled data from low renin subjects (Part-1) from the lorundrostat 25 mg BID, 50 mg QD and 100 mg QD cohorts.

### DETAILED DESCRIPTION OF THE INVENTION

### Methods of Treating Hypertension

This invention provides a method of treating hypertension in a hypertensive subject, the method administering to the subject, an amount of a diuretic and an amount of a CYP 11β2 beta hydroxylase inhibitor once or twice per day, wherein the amounts taken together are sufficient to treat hypertension in the hypertensive subject, preferably when taken in combination with other antihypertensive agents. In particular, the method is sufficient to elicit a safe and robust reduction in hypertension (>10 mmHg reduction in systolic blood pressure) in the hypertensive subject.

In embodiments of the invention, the diuretic and CYP 11β2 beta hydroxylase inhibitor are administered in a combined pharmaceutical composition.

In embodiments of the invention, the diuretic and CYP 11β2 beta hydroxylase inhibitor are administered concomitantly.

In embodiments of the invention, the amounts when taken together are effective to achieve a greater than additive therapeutic result in treating the subject.

In embodiments of the invention, the amount of CYP 11β2 beta hydroxylase inhibitor and the amount of diuretic when administered together is more effective to treat the subject than when each agent at the same amount is administered alone

This invention also provides a method of treating hypertension in a hypertensive subject who is taking at least one diuretic, the method comprising administering to the subject a CYP 11β2 beta hydroxylase inhibitor once or twice per day in an amount sufficient to thereby treat hypertension in the hypertensive subject.

In embodiments of the invention the diuretic is a thiazide diuretic.

In an embodiment, the hypertensive subject has a body mass index of at least 30, preferably more than 30, preferably 30-50, more preferably 30-40.

In an embodiment, the hypertensive subject is a male hypertensive subject having a waist-hip ratio above 0.90, or a female hypertensive subject having a waist-hip ratio above 0.85.

In an embodiment, the hypertensive subject has a serum leptin concentration of at least 30 ng/dL, preferably at least 35 ng/dL, more preferably at least 40 ng/dL, or more preferably 30-50 ng/dL, 30-45 ng/dL, or 35-50 ng/dL.

In embodiments, the hypertensive subject has:
(a) a plasma aldosterone concentration of greater than or equal to 6 ng/dL by immunoassay in said subject; and/or
(b) a plasma aldosterone concentration of greater than or equal to 1 ng/dL by LC-MS in said subject.

Plasma aldosterone concentration can be measured by standard, commercially available tests known in the art. Such measurements can be conducted by FDA-approved laboratories. See, e.g., Stowasser et al, Clin Biochem Rev, 31(2):39-56 (2010), citing Schirpenbach, et al. Clinical chemistry 52, no. 9 (2006): 1749-1755. Notably, the assays for measuring aldosterone reported in Schirpenbach, et al. are immunoassays. As reported in Guo et al. The Journal of Clinical Endocrinology & Metabolism 103, no. 11 (2018): 3965-3973, LC-MS assays have been shown to have a higher specificity. In an embodiment, a plasma aldosterone concentration of greater than or equal to 6 ng/dL as measured by an immunoassay such as ELISA corresponds to a plasma aldosterone concentration of greater than or equal to about 1 ng/dL as measured by LC-MS.

This invention provides a method of treating hypertension in a hypertensive subject in need thereof, the method comprising
(a) measuring
   (i) a systolic blood pressure of greater than 130 mmHg in said subject;
   (ii) a diastolic BP of greater than 80 mmHg in said subject; and
(b) selecting a subject who is taking at least one diuretic; and
(c) administering to said subject an effective amount of a CYP 11β2 beta hydroxylase inhibitor.

This invention provides a method of treating hypertension in a hypertensive subject in need thereof, the method comprising
(a) receiving an identification of the hypertensive subject as
   (i) having a systolic blood pressure of greater than 130 mmHg;
   (ii) having a diastolic BP of greater than 80 mmHg; and
   (iii) taking at least one diuretic; and
administering to said subject an effective amount of a CYP 11β2 beta hydroxylase inhibitor.

In an embodiment, the diuretic is a thiazide diuretic.

In an embodiment, step (a) further comprises measuring in said subject or receiving an identification of said subject having a body mass index (BMI) of at least 30, preferably more than 30, preferably 30-50, more preferably 30-40, or a waist-hip ratio above 0.90 if the hypertensive subject is male, or a waist-hip ratio above 0.85 if the hypertensive subject is female.

In an embodiment, step a) further comprises measuring in said subject or receiving an identification of said subject having a serum leptin concentration of at least 30 ng/dL, preferably at least 35 ng/dL, more preferably at least 40 ng/dL, or more preferably 30-50 ng/dL, 30-45 ng/dL, or 35-50 ng/dL.

In embodiments of this invention, step (a) further comprises:
(a) measuring a plasma aldosterone concentration of greater than or equal to 6 ng/dL in said subject by immunoassay; or
(b) measuring a plasma aldosterone concentration of greater than or equal to 1 ng/dL in said subject by LC-MS.

In embodiments of the invention, the hypertensive subject is taking or has taken a hypertension medication selected from an ACE inhibitor, an angiotensin receptor blocker, a calcium channel blocker, or a combination of two or more thereof. In embodiments of the invention, the hypertensive subject is taking or has taken at least two of said hypertension medications.

In embodiments of the invention, 50% or more of CYP 11β2 beta hydroxylase's activity is inhibited for 40-60% of a 24-hour period.

In embodiments of the invention, 50% or more of CYP 11β2 beta hydroxylase's activity is inhibited for between 10 to 14 hours of a 24-hour period.

In embodiments of the invention, the CYP 11β2 beta hydroxylase inhibitor reduces the serum aldosterone level of the subject by 50-90% relative to the subject's pre-drug level of serum aldosterone for a period not less than eight hours and not greater than 16 hours.

In embodiments of the invention, the CYP 11β2 beta hydroxylase inhibitor reduces the serum aldosterone level of the subject by 60-80% relative to the subject's pre-drug level of serum aldosterone for a period not less than eight hours and not greater than 16 hours.

In embodiments of the invention, the CYP 11β2 beta hydroxylase inhibitor allows serum aldosterone of the subject to return to the subject's pre-drug level of serum aldosterone or greater during the period between 16 and 24 hours after the dose is administered.

In embodiments of the invention, 50% or more of CYP 11β2 beta hydroxylase's activity is inhibited for between 1 and 16 hours, or preferably for between 3 and 8 hours, of a 24-hour period.

In embodiments, the CYP 11β2 beta hydroxylase inhibitor is administered once per day in an amount sufficient to:
(a) inhibit 50% or more of CYP 11β2 beta hydroxylase's activity for between 1 and 16 hours, preferably for between 3 and 8 hours;
(b) inhibit 60% or more of CYP 11β2 beta hydroxylase's activity for between 1 and 13 hours, preferably for between 2 and 6 hours;
(c) inhibit 70% or more of CYP 11β2 beta hydroxylase's activity for between 1 and 9 hours, preferably for between 2 and 5 hours;
(d) inhibit 80% or more of CYP 11β2 beta hydroxylase's activity for between 1 and 6 hours, preferably for between 1 and 3 hours; and/or
(e) inhibit 90% or more of CYP 11β2 beta hydroxylase's activity for between 0 and 3 hours, preferably for between 0 and 1 hour;
to thereby treat hypertension in the hypertensive subject.

In embodiments, the CYP 11β2 beta hydroxylase inhibitor is administered once per day in an amount sufficient to:
(a) inhibit 50% or more of CYP 11β2 beta hydroxylase's activity for between 1 and 20 hours, preferably for between 4 and 11 hours;
(b) inhibit 60% or more of CYP 11β2 beta hydroxylase's activity for between 1 and 17 hours, preferably for between 3 and 9 hours;
(c) inhibit 70% or more of CYP 11β2 beta hydroxylase's activity for between 1 and 15 hours, preferably for between 2.5 and 7 hours;
(d) inhibit 80% or more of CYP 11β2 beta hydroxylase's activity for between 1 and 10 hours, preferably for between 2 and 5 hours; and/or
(e) inhibit 90% or more of CYP 11β2 beta hydroxylase's activity for between 1 and 5 hours, preferably for between 0.5 and 2.5 hours;
to thereby treat hypertension in the hypertensive subject.

In embodiments of the invention, the CYP 11β2 beta hydroxylase inhibitor is administered to the subject once per day. In embodiments of the invention, the CYP 11β2 beta hydroxylase inhibitor is administered in the morning. In embodiments of the invention, the CYP 11β2 beta hydroxylase inhibitor is administered to the subject twice per day. In a preferred embodiment of the invention, the CYP 11β2 beta hydroxylase inhibitor is administered to the subject once per day in the morning.

In embodiments of the invention, the CYP 11β2 beta hydroxylase inhibitor:
(a) is administered daily for at least one week;
(b) is administered daily for at least two weeks;
(c) is administered daily for at least four weeks; or
(d) is administered daily for at least eight weeks.

In embodiments of the invention, the hypertensive subject's ambulatory systolic blood pressure is reduced by at least 10 mmHg, by 10-55 mmHg, by 10-50 mmHg, by 10-45 mmHg, by 10-40 mmHg, by 10-35 mmHg, by 10-30 mmHg, by 10-25 mmHg, by 10-20 mmHg, or by 10-15 mmHg, relative to the hypertensive subject's ambulatory systolic blood pressure prior to administration of the CYP 11β2 beta hydroxylase inhibitor, preferably relative to the hypertensive subject's ambulatory systolic blood pressure prior to administration of the CYP 11β2 beta hydroxylase inhibitor for a period of at least eight weeks.

In embodiments of the invention, the hypertensive subject's ambulatory diastolic blood pressure is reduced by at least 5 mmHg, by 5-25 mmHg, by 5-20 mmHg, or by 5-15 mmHg relative to the hypertensive subject's ambulatory diastolic blood pressure prior to administration of the CYP 11β2 beta hydroxylase inhibitor, preferably relative to the hypertensive subject's ambulatory diastolic blood pressure prior to administration of the CYP 11β2 beta hydroxylase inhibitor for a period of at least eight weeks.

In embodiments of the invention:
(a) the subject's office-measured systolic blood pressure is lowered relative to the subject's office-measured systolic blood pressure prior to administration of the CYP 11β2 beta hydroxylase inhibitor; and/or
(b) the subject's 24-hour ambulatory systolic blood pressure is lowered relative to the subject's ambulatory systolic blood pressure prior to administration of the CYP 11β2 beta hydroxylase inhibitor.

In embodiments of the invention:
(a) the subject's office-measured systolic blood pressure is lowered by at least 10 mmHg relative to the subject's office-measured systolic blood pressure prior to administration of the CYP 11β2 beta hydroxylase inhibitor; and/or
(b) the subject's ambulatory systolic blood pressure is lowered by at least 10 mmHg relative to the subject's ambulatory systolic blood pressure prior to administration of the CYP 11β2 beta hydroxylase inhibitor.

In embodiments of the invention:
(a) the subject's office-measured diastolic blood pressure is lowered relative to the subject's office-measured diastolic blood pressure prior to administration of the CYP 11β2 beta hydroxylase inhibitor;
(b) the subject's office-measured systolic and diastolic blood pressure is lowered relative to the subject's office-measured systolic and diastolic blood pressure prior to administration of the CYP 11β2 beta hydroxylase inhibitor;
(c) the subject's ambulatory systolic and diastolic blood pressure is lowered relative to the subject's ambulatory systolic and diastolic blood pressure prior to administration of the CYP 11β2 beta hydroxylase inhibitor; and/or
(d) the subject's systolic blood pressure is reduced to less than 130 mmHg and/or the subject's diastolic blood pressure is reduced to less than 80 mmHg.

In embodiments of the invention:
(a) the subject's ambulatory systolic blood pressure is lowered by at least 10 mmHg, and the subject's ambulatory diastolic blood pressure is lowered by at least 5 mmHg each relative to the subject's ambulatory systolic and diastolic blood pressure, respectively, prior to administration of the CYP 11β2 beta hydroxylase inhibitor;
(b) the subject's office-measured systolic blood pressure is lowered by at least 10 mmHg and the subject's office-measured diastolic blood pressure is lowered by at least 5 mmHg, each relative to the subject's office-measured systolic and diastolic blood pressure, respectively, prior to administration of the CYP 11β2 beta hydroxylase inhibitor; and/or
(c) the subject's systolic blood pressure is reduced to less than 130 mmHg and/or the subject's diastolic blood pressure is reduced to less than 80 mmHg.

In embodiments of the invention, the duration of inhibition of CYP 11β2 beta hydroxylase activity is sufficient to maintain a state of sodium and volume depletion in the hypertensive subject.

In embodiments of the invention, the method does not produce a persistent state of hyperkalemia or mild non-anion gap metabolic acidosis in the hypertensive subject.

In embodiments of the invention, the CYP 11β2 beta hydroxylase inhibitor does not substantially accumulate in the hypertensive subject, preferably wherein the lack of substantial accumulation of the CYP 11β2 beta hydroxylase inhibitor in the hypertensive subject allows for the hypertensive subject's aldosterone levels to return to pre-drug baseline within 24-48 hours of the CYP 11β2 beta hydroxylase inhibitor being administered, more preferably within 16-24 hours of the CYP 11β2 beta hydroxylase inhibitor being administered.

In embodiments of the invention, the hypertensive subject's potassium levels are generally maintained in a clinically normal range, preferably wherein the hypertensive subject's potassium levels are mildly elevated relative to the hypertensive subject's potassium levels prior to administration of the CYP 11β2 beta hydroxylase inhibitor, more preferably wherein the hypertensive subject's potassium levels are elevated by 0.35 mmol/L or less, more preferably wherein the hypertensive subject's potassium levels are maintained below a level of 5.5 mmol/L, more preferably wherein the hypertensive subject's potassium levels are maintained between 3.5 mEq/1 to 5.1 mEq/1.

In embodiments of the invention, the CYP 11β2 beta hydroxylase inhibitor is administered to the hypertensive subject in an amount which:
(a) suppresses aldosterone production in the subject;
(b) increases serum and/or plasma potassium levels in the subject; and/or
(c) increases plasma renin activity (PRA) in the subject.

In embodiments of the invention:
(a) serum and/or plasma aldosterone AUC-24 is reduced in the subject by at least 25% relative to the aldosterone levels in the subject prior to administration of the CYP 11β2 beta hydroxylase inhibitor;
(b) serum and/or plasma potassium levels in the subject are increased by at least 0.2 mMol/L relative to the serum and/or plasma potassium levels in the subject prior to administration of the CYP 11β2 beta hydroxylase inhibitor; and/or
(c) PRA in the subject is increased by at least 5 ng/ml/hr relative to the PRA in the subject prior to administration of the CYP 11β2 beta hydroxylase inhibitor.

In embodiments of the invention, the hypertensive subject's aldosterone level follows a substantially normal circadian rhythm.

In embodiments of the invention, the hypertensive subject's average systolic blood pressure during sleep is reduced (a) relative to the hypertensive subject's average systolic blood pressure during sleep prior to receiving the CYP 11β2 beta hydroxylase inhibitor, and/or (b) relative to the hypertensive subject's average daytime systolic blood pressure. In embodiments, the hypertensive subject's average systolic blood pressure during sleep is reduced:
(a) by at least 10%, by between 10% and 40%, by between 10% and 30%, or by between 10% and 20% relative to the hypertensive subject's average daytime systolic blood pressure; and/or
(b) by at least 8 mmHg, by at least 10 mmHg, by between 8 and 55 mmHg, by between 10 and 45 mmHg, or by between 10 and 25 mmHg, relative to the hypertensive sub-ject's average systolic blood pressure during sleep prior to receiving the CYP 11β2 beta hydroxylase inhibitor.

In embodiments of the invention, said CYP 11β2 beta hydroxylase inhibitor is selective for inhibition of CYP 11β2 beta hydroxylase activity relative to inhibition of CYP 11 β1 beta hydroxylase activity, preferably wherein the inhibition constant (Ki) for CYP 11 β1 beta hydroxylase divided by the Ki for CYP 11 β2 beta hydroxylase is greater than 100.

In embodiments of the invention, the CYP 11β2 beta hydroxylase inhibitor is administered to the hypertensive subject in an amount below the amount which causes the subject's serum and/or plasma 11-deoxycortisterone (11-DOC) levels to exceed 600 pmol/L, preferably below the amount which causes the subject's serum and/or plasma 11-DOC levels to exceed 400 pmol/L.

In embodiments of the invention, the CYP 11β2 beta hydroxylase inhibitor is administered to the hypertensive subject in an amount below the amount which causes an accumulation of 11-DOC above 0.1 ng/ml in the subject.

In embodiments of the invention, the CYP 11β2 beta hydroxylase inhibitor is administered to the hypertensive subject in an amount which does not cause a clinically meaningful upregulation of the subject's adrenocortical hormone synthesis.

In embodiments of the invention, the CYP 11β2 beta hydroxylase inhibitor is administered to the hypertensive subject in an amount which:
(a) does not cause a clinically meaningful reduction of the subject's serum and/or plasma cortisol levels, relative to the subject's serum and/or plasma cortisol levels prior to administration of the CYP 11β2 beta hydroxylase inhibitor;
(b) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or
(c) does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor.

In embodiments of the invention, the CYP 11β2 beta hydroxylase inhibitor is administered to the hypertensive subject in an amount:
(a) which does not cause a reduction of more than 20% in the subject's serum and/or plasma cortisol levels, relative to the subject's serum and/or plasma cortisol levels prior to administration of the CYP 11β2 beta hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
(b) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
(c) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

In embodiments of the invention, the CYP 11β2 beta hydroxylase inhibitor is a compound described in U.S. Pat. No. 10,029,993, the disclosure of which is incorporated by reference herein. In embodiments, the CYP11β2 beta hydroxylase inhibitor is a compound described in U.S. Pat. No. 10,329,263, the disclosure of which is incorporated by reference herein. In embodiments, the CYP11B2 beta hydroxylase inhibitor is a 1,2,4-triazine compound or a pharmaceutically acceptable salt thereof.

In embodiments, the CYP11β2 beta hydroxylase inhibitor is a compound of formula (I) or a pharmaceutically acceptable salt thereof:

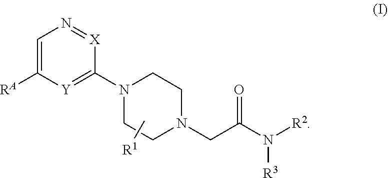

1) wherein X and Y represent any of the following (i) to (iii):
   (i) X is N, and Y is CH or CRY,
   (ii) X is CH, and Y is N, or
   (iii) X is CH, and Y is CH;
2) R^{Y} represents an alkyl group;
3) R^{A} represents a cycloalkyl group which may be substituted, a cycloalkenyl group which may be substituted, an aryl group which may be substituted, or a 6- to 10-membered monocyclic or bicyclic heteroaryl group which may be partially hydrogenated and may be substituted;
4) R¹ represents a hydrogen atom, or an alkyl group;
5) R2 represents an alkyl group which may be substituted, a cycloalkyl group which may be substituted, an aliphatic heterocyclic group which may be substituted, or a heteroaryl group which may be partially hydrogenated and may be substituted; and
6) R³ represents a hydrogen atom, or an alkyl group, or a pharmaceutically acceptable salt thereof.

In embodiments, said CYP 11β2 beta hydroxylase inhibitor is a compound of Formula (A) (also referred to herein as “Compound A”) or a pharmaceutically acceptable salt thereof:

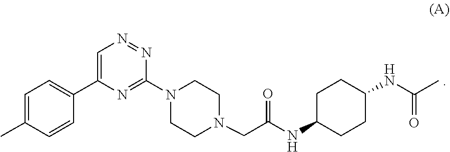 [SMILES: CC(=O)N[C@H]1CC[C@H](NC(=O)CN2CCN(c3nncc(-c4ccc(C)cc4)n3)CC2)CC1]

In embodiments, the CYP11β2 beta hydroxylase inhibitor is a pharmaceutically acceptable salt of the compound of Formula (A).

In embodiments, the CYP11β2 beta hydroxylase inhibitor is a monohydrobromide salt of the compound of Formula (A), i.e. Compound A HBr.

In embodiments, the CYP11β2 beta hydroxylase inhibitor is the free base form of the compound of Formula (A).

In embodiments, preferably wherein the CYP11β2 beta hydroxylase inhibitor is Compound A, more preferably Compound A HBr:
(a) between 5 mg and 100 mg of the CYP 11β2 beta hydroxylase inhibitor is administered orally twice a day, 12 hours apart;
(b) between 10 mg and 50 mg of the CYP 11β2 beta hydroxylase inhibitor is administered orally twice a day, 12 hours apart;
(c) between 5 mg and 100 mg of the CYP 11β2 beta hydroxylase inhibitor is administered orally once a day; or
(d) between 10 mg and 50 mg of the CYP 11β2 beta hydroxylase inhibitor is administered orally once a day.

In embodiments, preferably wherein the CYP11β2 beta hydroxylase inhibitor is Compound A, more preferably Compound A HBr:
(a) 12.5 mg of the CYP 11β2 beta hydroxylase inhibitor is administered orally twice a day, 12 hours apart;
(b) 25 mg of the CYP 11β2 beta hydroxylase inhibitor is administered orally twice a day, 12 hours apart;
(c) 12.5 mg of the CYP 11β2 beta hydroxylase inhibitor is administered orally once a day;
(d) 50 mg of the CYP 11β2 beta hydroxylase inhibitor is administered orally once a day; or
(e) 100 mg of the CYP 11β2 beta hydroxylase inhibitor is administered orally once a day.

In an embodiment, wherein the CYP11β2 beta hydroxylase inhibitor is Compound A and the diuretic is a thiazide diuretic:
(a) 12.5 mg of the CYP 11β2 beta hydroxylase inhibitor is administered orally once a day and the hypertensive subject experiences a placebo-adjusted reduction in systolic blood pressure of at least 5 mmHg, preferably by between 5 and 10 mmHg;
(b) 50 mg of the CYP 11β2 beta hydroxylase inhibitor is administered orally once a day and the hypertensive subject experiences a placebo-adjusted reduction in systolic blood pressure of at least 10 mmHg, preferably by between 10 and 15 mmHg; or
(c) 100 mg of the CYP 11β2 beta hydroxylase inhibitor is administered orally once a day and the hypertensive subject experiences a placebo-adjusted reduction in systolic blood pressure of at least 9 mmHg, preferably by between 9 and 15 mmHg.

In an embodiment, the CYP 11β2 beta hydroxylase inhibitor is a compound having the following structure, or a pharmaceutically acceptable salt thereof.

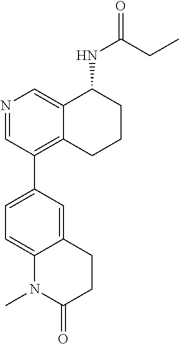 [SMILES: CCC(=O)N[C@@H]1CCCc2c(-c3ccc4c(c3)CCC(=O)N4C)cncc21]

In an embodiment, the CYP 11β2 beta hydroxylase inhibitor is a compound having the following structure:

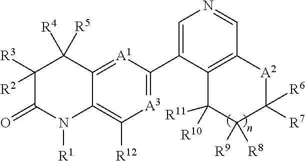

or a pharmaceutically acceptable salt thereof, wherein:
(a) R¹ is C₁-C₇-alkyl;
(b) R², R³, R⁴, and R⁵ are H;
(c) R⁶ is H, halogen or C₁-C₇-alkyl;
(d) R⁷, R⁸, R⁹, R¹⁰, and R¹¹ are H;
(e) R¹² is H or halogen;
(f) A¹ is CR;
(g) A² is NR¹⁴ or CR¹⁵R¹⁶;
(h) A³ is CR¹⁷;
(i) R¹³ is H or halogen;
(j) R¹⁴ is —(CR²⁰R²¹)_{q}—(CR²²R²³)_{f}—(CR²⁴R²⁵)ₚ—NR²⁶R²⁷, wherein the sum of q, r and p is at least 2;
(k) R¹⁵ is —(CR²⁰R²¹)_{q}—(CR²²R²³)_{f}—(CR²⁴R²⁵)ₚ—NR²⁶R²⁷;
(1) R¹⁶ is H;
(m) or R⁶ and R¹⁶ together with the carbon atoms to which they are attached form a double bond;
(n) R¹⁷, R², R²², R²³, R²⁴, R²⁵, and R²⁶ are H;
(o) R²⁷ is H, —S(O)₂R³¹, —C(O)R³¹ or —C(O)OR³¹, wherein in case R²⁶ is H and R²⁷ is H, then the sum of q, r and p is at least 1;
(p) R³¹ is C₁-C₇-alkyl, chloropyridinyl, hydroxyl-C₁-C₇-alkyl or C₃-C₈-cycloalkyl;
(q) n is zero or 1;
(r) p is zero or 1;
(s) q is zero or 1; and
(t) r is zero or 1.

Synthesis of these compounds is described in U.S. Pat. No. 9,353,081 B2, the entire contents of which are hereby incorporated by reference. In an embodiment, the CYP 11β2 beta hydroxylase inhibitor is a compound described in U.S. Pat. No. 9,353,081 B2, preferably a compound selective for inhibition of CYP 11β2 beta hydroxylase activity relative to inhibition of CYP 11 β1 beta hydroxylase activity, more preferably wherein the inhibition constant (Ki) for CYP 11 β1 beta hydroxylase divided by the Ki for CYP 11 β2 beta hydroxylase is greater than 100.

In an embodiment, the CYP 11β2 beta hydroxylase inhibitor is a compound having the following structure:

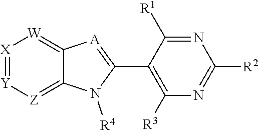

or a pharmaceutically acceptable salt thereof, wherein:
(a) A is N;
(b) W is CR6;
(c) X is CR6;
(d) Y is CR6;
(e) Z is CR6;
(f) R1 is hydrogen, halogen, cyano, acyl, alkyl, alkenyl, alkynyl, haloalkyl, alkoxy, haloalkoxy, cycloalkyl, cycloalkoxy, aryl, arylalkyl, heteroaryl, heteroarylalkyl, heterocycloalkyl, heterocycloalkylalkyl, NRaRb, NHSO2Rc, (CH2)nNRaRb, (CH2)nNHSO2Rd, (CH2)nNHCO2Rd, CO2Re, CORf, (CH2)nORf, or CReRfOH;
(g) R2 is hydrogen, cyano, acyl, alkyl, alkenyl, alkynyl, haloalkyl, alkoxy, haloalkoxy, cycloalkyl, cycloalkoxy, aryl, arylalkyl, heteroaryl, heteroarylalkyl, heterocycloalkyl, heterocycloalkylalkyl, NHSO2Rc, CH2NRaRb, CH2NHSO2Rd, CO2Re, CORf, CH2ORf, or CReRfOH;
(h) R3 is hydrogen, halogen, cyano, acyl, alkyl, alkenyl, alkynyl, haloalkyl, alkoxy, haloalkoxy, cycloalkyl, cycloalkoxy, aryl, arylalkyl, heteroaryl, heteroarylalkyl, heterocycloalkyl, heterocycloalkylalkyl, NRaRb, NHSO2Rc, (CH2)nNRaRb, (CH2)nNHSO2Rd, CO2Re, CORf, (CH2)nORf, or CReRfOH;
(i) each n is independently 1, 2, 3, 4, 5, 6, 7, 8, 9, or 10;
(j) R4 is alkyl, cycloalkyl, haloalkyl, or heteroalkyl;
(k) each occurrence of R6 is, independently, hydrogen, halogen, cyano, haloalkyl, alkyl, cycloalkyl, haloalkyl, alkoxy, haloalkoxy, alkylsulfonyl, or carboxyl; and
(l) each occurrence of Ra, Rb, Rc, Rd, Re, and Rf are, independently, hydrogen, acyl, alkyl, alkenyl, alkynyl, heteroalkyl, haloalkyl, cycloalkyl, heterocycloalkyl, aryl, heteroaryl, alkoxyalkyl, C(O)OC1-6 alkyl, C(O)C1-6 alkyl, a nitrogen protecting group when attached to a nitrogen atom, or an oxygen protecting group when attached to an oxygen atom; or Ra and Rb together with the atoms to which they are attached form a heterocycloalkyl ring; or Re and Rf together with the atoms to which they are attached form a cycloalkyl ring.

Synthesis of this compound is described in U.S. Pat. No. 10,538,511 the entire contents of which are hereby incorporated by reference. In an embodiment, the CYP 11β2 beta hydroxylase inhibitor is a compound described in U.S. Pat. No. 10,538,511, preferably a compound selective for inhibition of CYP 11β2 beta hydroxylase activity relative to inhibition of CYP 11 β1 beta hydroxylase activity, more preferably wherein the inhibition constant (Ki) for CYP 11 β1 beta hydroxylase divided by the Ki for CYP 11 β2 beta hydroxylase is greater than 100.

In an embodiment, the CYP 11β2 beta hydroxylase inhibitor is a compound having the following structure:

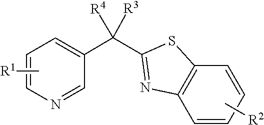

or a salt or enantiomer thereof, wherein:
(a) R¹ is one, two, or three independent halogen, haloalkyl, NO₂, CN, COOR⁵, SO₂R⁵, CONR⁵R⁶, SO₂NR⁵R⁶, NR⁵R⁶, OR⁵, alkyl, alkenyl, alkynyl, cycloalkyl, heteroalkyl or aryl;
(b) R² is one or more independent halogen, haloalkyl, NO₂, CN, COOR⁵, SO₂R⁵, CONR⁵R⁶, SO₂NR⁵R⁶, NR⁵R⁶, OR⁵, alkyl, alkenyl, alkynyl, cycloalkyl, heteroalkyl, or aryl;
(c) R³ is alkyl, alkenyl or alkynyl, any of which is further substituted by one or more R⁷;
(d) R⁴ is NR⁵R⁶, SR⁵, or OR⁵;
(e) R⁵ and R⁶ are independently hydrogen, alkyl, aryl or haloalkyl;
(f) R⁷ is H, halogen, alkyl, haloalkyl, NO₂, CN, COOR⁸, SO₂R⁸, CONR⁸R⁹, SO₂NR⁸R⁹, NR⁸R⁹, or OR⁸; and
(g) R⁸ and R⁹ are independently hydrogen, alkyl or haloalkyl,
(h) and wherein the compound is not benzo[d]thiazol-2-yl(pyridin-3-yl)methanol.

Synthesis of this compound is described in U.S. Pat. No. 10,287,282 the entire contents of which are hereby incorporated by reference. In an embodiment, the CYP 11β2 beta hydroxylase inhibitor is a compound described in U.S. Pat. No. 10,287,282, preferably a compound selective for inhibition of CYP 11β2 beta hydroxylase activity relative to inhibition of CYP 11 β1 beta hydroxylase activity, more preferably wherein the inhibition constant (Ki) for CYP 11 β1 beta hydroxylase divided by the Ki for CYP 11 β2 beta hydroxylase is greater than 100.

In embodiments of the invention, the hypertensive subject has a plasma renin activity less than or equal to 1 ng/mL/hour.

In embodiments of the invention, the hypertensive subject has a plasma renin activity less than or equal to 0.6 ng/mL/hour.

In embodiments of the invention, the hypertensive subject has a plasma renin activity less than or equal to 4 ng/mL/hour.

In embodiments of the invention, the hypertensive subject has a plasma renin activity less than or equal to 3 ng/mL/hour.

In embodiments of the invention, the hypertensive subject has a plasma renin activity less than or equal to 2 ng/mL/hour.

In embodiments of the invention, the hypertensive subject has a plasma aldosterone concentration of greater than or equal to 6 ng/dL as measured by an immunoassay.

In embodiments of the invention, the hypertensive subject has a plasma aldosterone concentration of greater than or equal to 1 ng/dL as measured by LC-MS.

In a preferred embodiment of the invention, the hypertensive subject has a plasma renin activity less than or equal to 1 ng/mL/hour and a plasma aldosterone concentration of greater than or equal to 6 ng/dL as measured by an immunoassay. In a preferred embodiment of the invention, the hypertensive subject has a plasma renin activity less than or equal to 1 ng/mL/hour and a plasma aldosterone concentration of greater than or equal to 1 ng/dL as measured by LC-MS. In a further preferred embodiment, this hypertensive subject is taking or has taken a hypertension medication selected from a diuretic, an ACE inhibitor, an angiotensin receptor blocker, a calcium channel blocker, or a combination of two or more thereof.

Alternatively, in embodiments wherein the hypertensive subject is not taking a hypertension medication selected from a diuretic, an ACE inhibitor, an angiotensin receptor blocker, a calcium channel blocker, in an embodiment the hypertensive subject has a plasma renin activity less than or equal to 0.6 ng/mL/hour and has a plasma aldosterone concentration of greater than or equal to 6 ng/dL as measured by an immunoassay or greater than or equal to 1 ng/dL as measured by LC-MS.

In embodiments of the invention, the hypertensive subject has secondary hypertension, preferably primary aldosteronism. In other embodiments of the invention, the hypertensive subject does not have primary aldosteronism, preferably wherein the hypertensive subject has primary hypertension.

This invention also provides a method of identifying a subject for hypertension treatment with a CYP 11β2 beta hydroxylase inhibitor, the method comprising:
(a) measuring:
   (i) a systolic blood pressure of greater than 130 mmHg in said subject; and
   (ii) a diastolic BP of greater than 80 mmHg in said subject; and
(b) selecting a subject who is taking at least one diuretic;
thereby identifying said subject for hypertension treatment with a CYP 11β2 beta hydroxylase inhibitor.

In embodiments of the invention, the diuretic is a thiazide diuretic.

In embodiments of the invention, step (a) further comprises measuring a body mass index (BMI) of at least 30, preferably more than 30, preferably 30-50, more preferably 30-40, in said subject, or measuring a waist-hip ratio above 0.90 in said subject if the subject is male, or measuring a waist-hip ratio above 0.85 in said subject if the subject is female.

In embodiments of the invention, step (a) further comprises measuring in said subject a serum leptin concentration of at least 30 ng/dL, preferably at least 35 ng/dL, or more preferably at least 40 ng/dL.

In embodiments of the invention, step a) further comprises:
(a) measuring a plasma aldosterone concentration of greater than or equal to 6 ng/dL by immunoassay in said subject; or
(b) measuring a plasma aldosterone concentration of greater than or equal to 1 ng/dL by LC-MS in said subject.

In preferred embodiments of the invention, the subject identified for treatment with a CYP 11β2 beta hydroxylase inhibitor experiences an average drop of at least 10 mmHg, 10-55 mmHg, 10-50 mmHg, 10-45 mmHg, 10-40 mmHg, 10-35 mmHg, 10-30 mmHg, 10-25 mmHg, 10-20 mmHg, or 10-15 mmHg in systolic blood pressure when treated with the CYP 11β2 beta hydroxylase inhibitor relative to hypertensive subject's average systolic blood pressure prior to treatment with the CYP 11β2 beta hydroxylase inhibitor. In a particularly preferred embodiment the ambulatory systolic blood pressure of the subject identified for treatment with a CYP 11β2 beta hydroxylase inhibitor is reduced to less than 130 mmHg when treated with the CYP 11β2 beta hydroxylase inhibitor and/or the average ambulatory diastolic blood pressure of the subject identified for treatment with a CYP 11β2 beta hydroxylase inhibitor is reduced to less than 80 mmHg when treated with the CYP 11β2 beta hydroxylase inhibitor.

### Compositions

This invention also provides pharmaceutical compositions, packages, and unit dosage forms for use in any one of the methods described herein.

Accordingly, this invention provides a package comprising:
(a) a first pharmaceutical composition comprising an amount of a compound of Formula (A) or a pharmaceutically acceptable salt thereof:

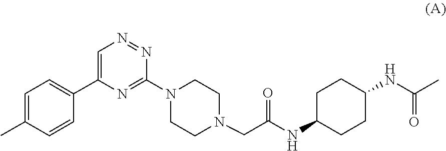 [SMILES: CC(=O)N[C@H]1CC[C@H](NC(=O)CN2CCN(c3nncc(-c4ccc(C)cc4)n3)CC2)CC1]

and a pharmaceutically acceptable carrier;
(b) a second pharmaceutical composition comprising an amount of a thiazide diuretic and a pharmaceutically acceptable carrier; and
(c) instructions for use of the first and second pharmaceutical compositions together to treat a subject afflicted with hypertension.

This invention also provides a compound of Formula (A) or a pharmaceutically acceptable salt thereof:

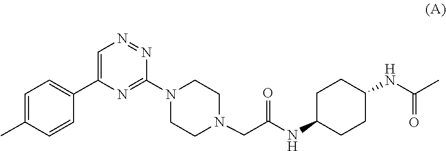 [SMILES: CC(=O)N[C@H]1CC[C@H](NC(=O)CN2CCN(c3nncc(-c4ccc(C)cc4)n3)CC2)CC1]

for use as an add-on therapy or in combination with a thiazide diuretic in treating a subject afflicted with hypertension.

This invention also provides a pharmaceutical composition comprising
(a) an amount of the compound of Formula (A) or a pharmaceutically acceptable salt thereof:

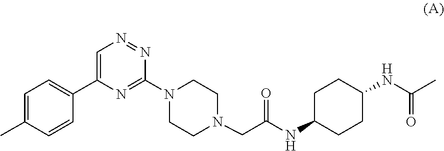 [SMILES: CC(=O)N[C@H]1CC[C@H](NC(=O)CN2CCN(c3nncc(-c4ccc(C)cc4)n3)CC2)CC1]

(b) and an amount of a thiazide diuretic for use in treating a subject afflicted with hypertension, wherein the compound of Formula (A) and the thiazide diuretic are administered simultaneously, contemporaneously or concomitantly.

This invention also provides a pharmaceutical composition comprising
(a) an amount of the compound of Formula (A) or a pharmaceutically acceptable salt thereof:

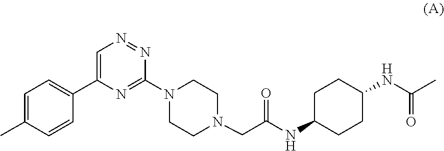 [SMILES: CC(=O)N[C@H]1CC[C@H](NC(=O)CN2CCN(c3nncc(-c4ccc(C)cc4)n3)CC2)CC1]

(b) and an amount of a thiazide diuretic
wherein the compound of Formula (A) and thiazide diuretic are each present in amounts which together are effective to treat hypertension in a hypertensive subject.

This invention also provides a pharmaceutical composition comprising an amount of the compound of Formula (A) or a pharmaceutically acceptable salt thereof:

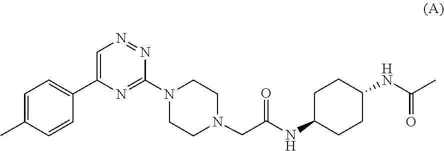 [SMILES: CC(=O)N[C@H]1CC[C@H](NC(=O)CN2CCN(c3nncc(-c4ccc(C)cc4)n3)CC2)CC1]

for use in treating a subject afflicted with hypertension as an add-on therapy or in combination with a thiazide diuretic.

This invention also provides a therapeutic package for dispensing to, or for use in dispensing to, a subject afflicted with hypertension, which comprises:
(a) one or more unit doses, each such unit dose comprising:
   (i) an amount of the compound of Formula (A) or a pharmaceutically acceptable salt thereof:

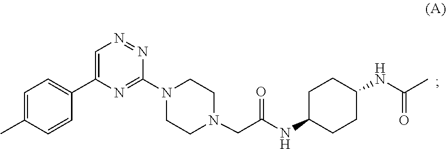 [SMILES: CC(=O)N[C@H]1CC[C@H](NC(=O)CN2CCN(c3nncc(-c4ccc(C)cc4)n3)CC2)CC1]

(ii) an amount of a thiazide diuretic,
wherein the respective amounts of said compound of Formula (A) and said thiazide diuretic in said unit dose are effective, upon concomitant administration to said subject, to treat the subject, and
(b) a finished pharmaceutical container therefor, said container containing said unit dose or unit doses, said container further containing or comprising labeling directing the use of said package in the treatment of said subject.

This invention also provides a pharmaceutical composition in unit dosage form, useful in treating a subject afflicted with hypertension, which comprises:
(a) an amount of the compound of Formula (A) or a pharmaceutically acceptable salt thereof:

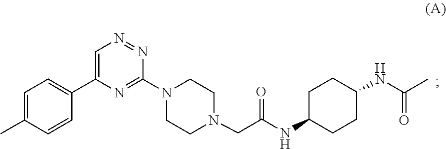 [SMILES: CC(=O)N[C@H]1CC[C@H](NC(=O)CN2CCN(c3nncc(-c4ccc(C)cc4)n3)CC2)CC1]

(b) an amount of a thiazide diuretic,
wherein the respective amounts of said compound of Formula (A) and said thiazide diuretic in said composition are effective, upon concomitant administration to said subject of one or more of said unit dosage forms of said composition, to treat the subject.

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

“Hypertension”, also called high blood pressure, is blood pressure that is higher than normal. In 2017, the American College of Cardiology and the American Heart Association published guidelines for hypertension management and defined hypertension as a blood pressure at or above 130 mmHg systolic blood pressure, 80 mmHg diastolic blood pressure. Stage 1 hypertension is defined as a blood pressure of 130-139 mmHg systolic blood pressure, 80-89 mmHg diastolic blood pressure, while stage 2 hypertension is defined as a blood pressure of greater than 140 mmHg systolic blood pressure, 90 mmHg diastolic blood pressure. As used herein, “hypertension” includes both stages 1 and 2 of hypertension unless indicated to the contrary. In an embodiment, the hypertensive subject has stage 1 hypertension. In another embodiment, the hypertensive subject has stage 2 hypertension. Hypertension includes high blood pressure that is multi-factorial and doesn't have one distinct cause (primary hypertension), and high blood pressure that has a direct cause (secondary hypertension). As used herein, “hypertension” includes both primary and secondary hypertension unless indicated to the contrary. In embodiments, the hypertensive subject has primary hypertension. In other embodiments, the hypertensive subject has secondary hypertension. Primary aldosteronism (hyperaldosteronism), the most common form of secondary hypertension, is a condition that occurs when the adrenal glands produce too much aldosterone. In embodiments where the hypertensive subject has secondary hypertension, the subject has primary aldosteronism.

“CYP11β2”, “Cyp11β2” or “CYP11β2 beta hydroxylase” is a cytochrome P450 enzyme, encoded by the CYP11B2 gene in humans, which catalyzes a series of reactions leading from 11-deoxycorticosterone (i.e., an aldosterone precursor) to aldosterone. Thus, it is referred to in the art as “aldosterone synthase.” Cyp11B2 is mainly expressed in an adrenal cortex spherical layer and a level of plasma aldosterone is regulated by enzymatic activity of Cyp11B2 present in the adrenal gland. Aldosterone is expressed in other tissues, such as cardiovascular, kidney, adipose, and brain.

“CYP111”, “Cyp11B1” or “CYP11β1 beta hydroxylase” is a cytochrome P450 enzyme, encoded by the CYP11B1 gene in humans, which is involved in the biosynthesis of adrenal corticosteroids. It is referred to in the art as “steroid 11β-hydroxylase.”

An “inhibitor” refers to a compound (e.g. compounds described herein) that reduces activity when compared to a control, such as absence of the compound or a compound with known inactivity. An inhibitor can be a small molecule inhibitor, an antibody inhibitor, a protein inhibitor, a biomolecule inhibitor, a natural ligand, and the like. An “inhibitor” may be in the form of a pharmaceutically acceptable salt, e.g. of the compounds described herein.

As used herein “Compound A” refers to the disubstituted 1,2,4-Triazine compound which is represented by Formula (A):

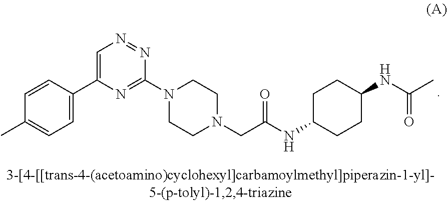 [SMILES: CC(=O)N[C@H]1CC[C@H](NC(=O)CN2CCN(c3nncc(-c4ccc(C)cc4)n3)CC2)CC1]

“Compound A HBr” refers to the hydrobromide (HBr) salt of Compound A. Weights and/or strengths of “Compound A HBr,” and “the compound” of the invention refer to the weight of the free base (i.e. Compound A) in the HBr salt and not the weight of the HBr salt.

Compound A and pharmaceutically acceptable salts thereof can be made by processes described, for example, in U.S. Pat. No. 10,029,993 and European Publication No. 3549935, the disclosures of which are incorporated by reference herein in their entirety.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may be a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is not prophylactic treatment.

A “diuretic” refers to a hypertension medication that increases the production of urine, thereby increasing the amount of water and salt eliminated from the body. The diuretic can be a carbonic anhydrase inhibitor, a loop diuretic, a potassium-sparing diuretic, a thiazide diuretic, or any other diuretic known in the art. Exemplary carbonic anhydrase inhibitors include acetazolamide, brinzolamide, dorzolamide, dichlorphenamide, ethoxaolamide, zoniamide, indisulam, and methazolamide. Exemplary loop diuretics include bumatenide, ethacrynic acid, torsemide, and furosemide. Exemplary potassium-sparing diuretics include epelerenone, triamterene, spironolactone, and amiloride. Exemplary thiazide diuretics include indapamide, hydrochlorothiazide, chlorthalidone, metolazone, methyclothiazide, chlorothiazide, methylclothiazide, metolazone, bendroflumethiazide, polythiazide, and hydroflumethiazide. Other diuretics include pamabrom and mannitol.

An “angiotensin-converting enzyme inhibitor” or “ACE inhibitor” refers to a hypertension medication that block angiotensin I from being converted to angiotensin II, thereby dilating blood vessels and lowering blood pressure. Exemplary ACE inhibitors include benazepril, zofenopril, perindopril, trandolapril, captopril, enalapril, lisinopril, and ramipril.

An “angiotensin receptor blocker” or “angiotensin II inhibitor” refers to a hypertension medication that blocks the receptor binding of angiotensin II, thereby dilating blood vessels and lowering blood pressure. Exemplary angiotensin receptor blockers include eprosartan, olmesartan, valsartan, candesartan, losartan, telmisartan, irbesartan, valsartan, and azilsartan medoxomil.

A “calcium channel blocker” refers to hypertension medication that can block calcium from entering the cells of the heart and arteries via a calcium channel, thereby lowering blood pressure. A calcium channel blocker can be a dihydropyridine calcium channel blocker, a phenylalkylamine calcium channel blocker, a benzothiazepine calcium channel blocker, a nonselective calcium channel blocker, or any other calcium channel blocker known in the art. Dihydropyridine calcium channel blockers include amoldipine, aranidipine, azelnidipine, bamidipine, benidipine, cilnidine, clevidipine, efonidipine, felodipine, isradipine, lacidipine, lercanidipine, manidipine, nicardipine, nifedipine, nilvadipine, nimodipine, nisoldipine, nitrendipine, and pranidipine. Phenylalkylamine calcium channel blockers include fendiline, gallipamil, and verapamil. Benzothiazepine calcium channel blockers include diltiazem. Nonselective calcium channel blockers include mibefradil, bepridil, flunarizine, fluspirilene, and fendiline. Other calcium channel blockers include gabapentin, pregabalin, and ziconotide.

A “normal circadian rhythm” for aldosterone level follows a diumal pattern wherein the nadir is in the late evening and the peak is in the early morning, pre-arousal. In an embodiment, the hypertensive subject's aldosterone level follows a substantially normal circadian rhythm. In one example of such an embodiment, the CYP 11β2 beta hydroxylase inhibitor of the invention, when dosed once daily in the morning after waking, suppresses the abnormally elevated production of Aldosterone during waking hours. In the early evening suppression of Aldosterone production starts to wane and the normal increase in serum Aldosterone returns towards normal by dawn, as it would under normal circumstances. The circadian rhythm of aldosterone in normal subjects and subjects with primary aldosteronism is described in Kem, David C., et al. “Circadian rhythm of plasma aldosterone concentration in patients with primary aldosteronism.” The Journal of clinical investigation 52.9 (1973): 2272-2277, the contents of which are specifically incorporated by reference herein.

This invention provides methods that reduce a hypertensive subject's systolic blood pressure “during sleep”. In this context, “during sleep” refers to the period of sleep in the hypertensive subject's normal sleep/wake cycle. In other words, “during sleep” refers to the hypertensive subject's approximately seven to nine hours of sleep (typically at night) that occurs daily in between their approximately 15 to 17 hours of wakefulness and not to any short period of sleep that may occur outside of the sleep phase of the subject's normal sleep/wake cycle (i.e. naps). The blood pressure of non-hypertensive individuals typically dips during sleep, with approximately 10% to 15% lower blood pressure values during sleep relative to during wakefulness. In contrast, hypertensive subjects may experience a smaller dip in blood pressure during sleep or may not experience any dip in blood pressure at all. Thus, methods of the invention help hypertensive subjects restore the dip in blood pressure that normal, non-hypertensive subjects experience during sleep.

A subject's “pre-drug level” of serum aldosterone refers to the subject's level of serum aldosterone, at the same time of day, in the absence of being treated with the CYP 11β2 beta hydroxylase inhibitor. As discussed above, aldosterone level follows a diurnal pattern where the nadir is in the late evening and the peak is in the early morning, pre-arousal. Therefore, in embodiments where the dose of CYP 11β2 beta hydroxylase inhibitor reduces the serum aldosterone level of the subject by a certain percentage relative to their “pre-drug level”, the reduction in serum aldosterone is measured relative to the same subject's serum aldosterone level in the absence of administration of the CYP 11β2 beta hydroxylase inhibitor at the same time of day. For example, the subject's serum aldosterone level at 11 AM when administered a CYP 11β2 beta hydroxylase inhibitor would be measured relative to that same subject's serum aldosterone level at 11 AM prior to any administration of the CYP 11β2 beta hydroxylase inhibitor.

### General

For the foregoing embodiments, each embodiment disclosed herein is contemplated as being applicable to each of the other disclosed embodiment.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1

A randomized, double-blinded, placebo-controlled, dose-ranging, multicenter study was conducted to evaluate the effect of orally administered Compound A HBr on blood pressure for the treatment of hypertension in male and female subjects ≥18 years of age.

### Study Design

The study consists of two parts. For enrollment into Part 1 of the study, a subject's value of plasma renin activity (PRA) must be ≤1 ng/mL/h based on morning measurement. If the value of PRA >1 ng/mL/h based on morning measurement, then subjects may be eligible to enter Part 2 of the study.

For Part 1, 163 enrolled subjects ≥18 years of age were randomized into 6 equal treatment groups (1:1:1:1:1:1) to 12.5 mg BID, 25 mg BID, 12.5 mg QD, 50 mg QD, 100 mg QD, or placebo. After a review of interim clinical data, the 2 lowest dose levels (12.5 mg QD and 12.5 mg BID) were stopped for future randomization due to a lack of consistent meaningful reduction of blood pressure, but the patients randomized to that point remained in the study through completion. Thus, after the review of interim clinical data, subjects were randomized into 4 equal treatment groups (1:1:1:1) to 25 mg BID, 50 mg QD, 100 mg QD, or placebo.

For Part 2, 36 enrolled subjects ≥18 years of age were randomized (5:1) to either 100 mg QD Compound A HBr or placebo such that the Compound A HBr treatment group consisted of approximately 30 subjects and the placebo treatment group will consist of approximately 6 subjects.

Subjects orally administered the assigned study drug (Compound A HBr or placebo) according to the assigned dosing regimen for 8 weeks beginning on Study Day 1. All subjects in Part 1 (regardless of dosing group) received BID dosing to preserve the integrity of the blind; active drug is administered as the morning dose for all QD dose groups. Subjects returned to the research facility or were seen by the clinical investigator or approved home health care professional at the end of Study Weeks 1, 2, 3, 4, 5, 6, 7, and 8 (±2 days) for protocol-defined efficacy and safety assessments and procedures, assessment of adverse events (AEs), and confirmation of compliance with study drug usage. Subjects also completed a telephone visit and blood pressure (BP) check at home approximately 3 days post last dose of study drug. Subjects attended up to 14 full clinic visits, including a pre-Screening visit, a Screening/start of Placebo Run-in visit, a second visit during Placebo Run-in, a clinic visit to initiate the ABPM procedure, a Randomization visit, 8 weekly visits during double-blind treatment, and an end-of-study visit scheduled 4 weeks after the last study treatment for final efficacy and safety assessments.

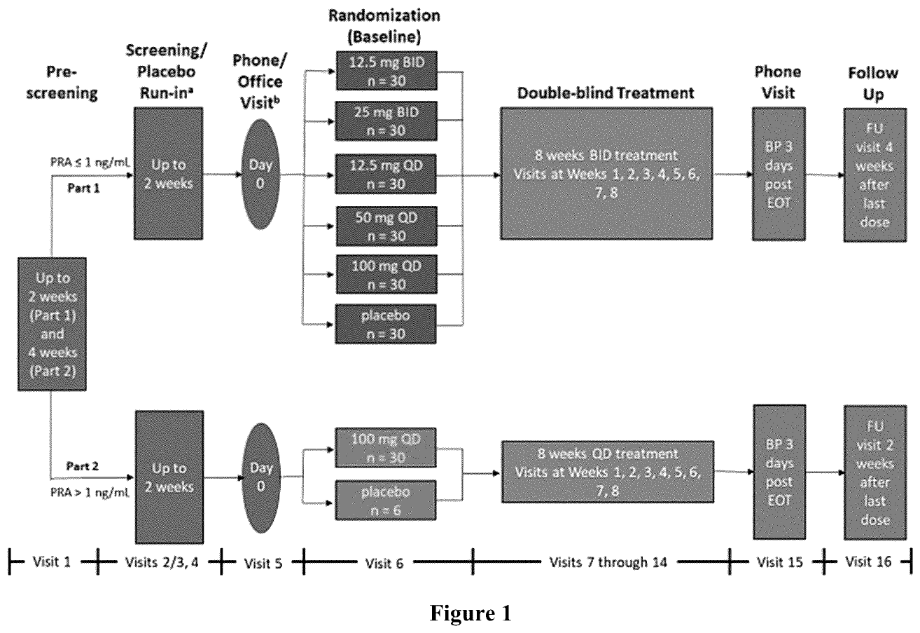

A schematic of the study design is shown in FIG. 1.

### Automated Office Blood Pressure (AOBP) Procedure

An automated oscillometric sphygmomanometer device was used to measure the subjects' systolic and diastolic blood pressure in office after approximately 5 minutes of rest in the seated position.

### 24-Hour Ambulatory Blood Pressure Monitoring (ABPM) Procedure

Ambulatory blood pressure monitoring was accomplished with an ambulatory blood pressure monitoring device that consists of a blood pressure cuff worn on a subject's arm attached to a small recording device that is typically attached to the subject's belt or waistband.

The ABPM device is worn for 24 hours. Throughout that period, the device records the subject's blood pressure at regular intervals, during the subject's routine daily activities and while they are sleeping. The ABPM thus provides a complete record of the subject's blood pressure over a 24-hour period.

24-hour ABPM was measured in the clinic at baseline and Study Week 7. If, for any reason, the ABPM procedure was deemed a failure at the end of Study Week 7, it may have been repeated at Study Week 8 and therefore no imputation was employed regardless of use of rescue medications. Additionally, ABPM was also collected at the end of Study Week 4 in Part 2. If a repeat test was performed, it supersedes the original test results for that visit.

Specific derived variables based on ABPM measurements include mean 24-hour, mean Daytime, and mean Nighttime of SBP, DBP, and heart rate.

Change from baseline to Week 7 in 24-hour mean SBP (and DBP) based on ABPM will be analyzed using an ANCOVA with a term for treatment group and a baseline mean 24-hour value as a covariate.

The nighttime dip is defined as

100% x(mean daytime SBP−mean nighttime SBP)/mean daytime SBP

It is expressed as a percentage and is summarized by treatment group and visit using descriptive statistics. In addition, the number and percentage of subjects with nighttime dip in each of the dipper categories (Bloomfield & Park, 2015) is presented by treatment group and visit. The categories are:
(a) <10%
(b) 10-20% inclusive
(c) >20%

### Eligibility Criteria

### Inclusion Criteria

The study was conducted using subjects meeting the following inclusion criteria:
(a) Male and nonpregnant, nonlactating female subjects ≥18 years of age.
(b) Automated office blood pressure (AOBP) with systolic blood pressure (SBP) ≥130 mm Hg
(c) Background antihypertensive treatment of ≥2 drugs
(d) Serum cortisol ≥18 mcg/dL

### Exclusion Criteria

Subjects were excluded from the study if they met any of the following exclusion criteria:
(a) Concomitant use of epithelial sodium channel inhibitors or mineralocorticoid receptor antagonists
(b) Subjects with hypokalemia
(c) Subjects with hyperkalemia
(d) Subjects with serum cortisol <3 mcg/dL
(e) Subjects with serum sodium <135 mEq/L
(f) Subjects with estimated glomerular filtration rate <60 mL/min/1.73 m2
(g) Subjects with type 1 or uncontrolled (hemoglobin A1c ≥9%) type 2 diabetes mellitus
(h) Subjects with body mass index >40 kg/m²
(i) Subjects with unstable angina
(j) Subjects with SBP ≥175 mm Hg or diastolic blood pressure (DBP) ≥100 mm Hg for Part 1 and SBP ≥160 mm Hg or DBP ≥100 mm Hg for Part 2 at Pre-Screening, Screening/Start of Placebo Run-in, or Randomization
(k) Subjects with a decrease in SBP ≥20 mm Hg or DBP ≥10 mm Hg from sitting to standing position at screening
(l) Subjects who, in the opinion of the investigator, have suspected nonadherence to antihypertensive treatment
(m) Subjects who, in the opinion of the investigator, have any major medical illness or symptoms
(n) Subjects who, in the opinion of the investigator, have any acute or chronic medical or psychiatric condition
(o) Subjects undergoing treatment with any of the following medications:
   (i) Topical corticoids
   (ii) Sympathomimetic decongestants
   (iii) Theophylline
   (iv) Phosphodiesterase type 5 inhibitors
   (v) NSAIDs
   (vi) Intramuscular steroids
   (vii) Estrogen
   (viii) Cytochromes
   (ix) Strong CYP3A and CYP3A4 inducers
(p) Subjects with known hypersensitivity to Compound A HBr or any of the excipients
(q) Subjects who are night-shift workers

### Arms and Interventions

The study contained the following arms and corresponding interventions:

TABLE 1

| Arm | Intervention* |
|---|---|
| Placebo Comparator: Placebo | Other: Placebo (Part I) |
| (Part I) | |
| Placebo tablet(s) by mouth | Placebo tablet(s) by mouth once |
| once or twice daily. | or twice daily. |
| Experimental: Dose 1 (Part I) | Drug: Compound A HBr (Part I) |
| Compound A HBr tablet(s) by | Tablet(s) containing 12.5 mg of |
| mouth once daily. | Compound A HBr by mouth once daily. |
| Experimental: Dose 2 (Part I) | Drug: Compound A HBr (Part I) |
| Compound A HBr tablet(s) | Tablet(s) containing 12.5 mg of |
| by mouth twice daily. | Compound A HBr by mouth twice daily. |
| Experimental: Dose 3 (Part I) | Drug: Compound A HBr (Part I) |
| Compound A HBr tablet(s) by | Tablet(s) containing 25 mg of |
| mouth twice daily. | Compound A HBr by mouth twice daily. |
| Experimental: Dose 4 (Part I) | Drug: Compound A HBr (Part I) |
| Compound A HBr tablet(s) by | Tablet(s) containing 50 mg of |
| mouth once daily. | Compound A HBr once daily. |
| Experimental: Dose 5 (Part I) | Drug: Compound A HBr (Part I) |
| Compound A HBr tablet(s) by | Tablet(s) containing 100 mg of |
| mouth once daily. | Compound A HBr once daily. |
| Placebo Comparator: Placebo | Other: Placebo (Part II) |
| (Part II) | |
| Placebo tablet(s) by mouth | Placebo tablet(s) by mouth once daily. |
| once daily. | |
| Experimental: Dose (Part II) | Drug: Compound A HBr (Part II) |
| Compound A HBr tablet(s) by | Tablet(s) containing 100 mg of |
| mouth once daily. | Compound A HBr by mouth once daily. |

### Study Endpoints

### Primary Endpoint

The primary endpoint is the change in office-measured (mean of last 2 of 5 unattended measurements using an automated oscillometric sphygmomanometer device after approximately 5 minutes of rest in the seated position) systolic blood pressure (SBP) from baseline to the end of Study Week 8.

### Secondary Endpoint

Secondary endpoints of this study were:
(a) Change in 24-hour ambulatory blood pressure monitoring (ABPM) parameters (systolic and diastolic) from baseline to the end of Study Week 7.
(b) Change in office-measured SBP from baseline to the end of Study Weeks 1, 2, 3, 4, 5, 6, and 7.
(c) Change in office-measured diastolic blood pressure (DBP) from baseline to the end of Study Weeks 1, 2, 3, 4, 5, 6, 7, and 8.
(d) Proportion of subjects who achieve office-measured BP of ≤130/80 mm Hg by the end of Study Week 8.

### Pharmacodynamic Endpoints

Pharmacodynamics endpoints of this study were:
(a) Change in plasma 11-deoxycortisol and PRA from baseline to the end of Study Week 4 and to end of follow up (i.e. end of Study Week 12 for Part 1 and end of Study 10 for Part 2).
(b) Change in serum aldosterone, cortisol, and 11-deoxycorticosterone concentration from baseline to the end of Study Week 4 and to end of follow-up.

### Pharmacokinetic Endpoints

Pharmacokinetic endpoints of this study are PK parameters, including, of area under the plasma concentration versus time curve (AUC), maximum plasma concentration (Cmax), time to maximum concentration (Tmax), and half-life (t_{1/2}) will be summarized descriptively for Randomization (baseline) and Study Weeks 1, 4, and 8

### Safety Endpoints

Safety endpoints of this study are:
(a) Incidence and severity of all spontaneously reported adverse events (AEs)
(b) Changes in vital signs (standing SBP, standing DBP, body temperature, heart rate, and respiratory rate)
(c) Changes in electrocardiogram parameters (including cardiac intervals: PR, QRS, QT, and corrected QT interval using Fridericia's formula)
(d) Changes in clinical laboratory assessments (hematology, chemistry, coagulation, and urinalysis)
(e) Change in office-measured SBP from Study Week 8 (end-of-treatment period to end of follow up (i.e. end of Study Week 12 for Part 1 and end of Study 10 for Part 2).

### Analysis Methodology

The following analysis sets are defined in this study:

### Full Analysis Set (FAS)

The FAS includes all randomized subjects who have received at least 1 dose of randomized study treatment (MLS-101 or placebo). The FAS will be the primary set for efficacy analyses. In analyses performed on the FAS, unless otherwise specified, subjects will be analyzed according to the randomized study treatment group.

### Per Protocol Set (PPS or PP)

The Per Protocol Set includes all subjects in the FAS who have completed the Study Week 8 visit without any major protocol violations that could influence the validity of the data for the primary efficacy evaluations. In the analyses based on PPS, subjects will be analyzed according to the randomized study treatment group. All criteria to exclude subjects from the PPS will be made based on a blinded review of the data prior to the unblinding of the study.

A subject may be excluded from the Per Protocol Analysis Set if any of the following criteria are met:
(a) Not meeting Inclusion/Exclusion criteria
(b) Use of prohibited medications. Subjects using rescue medications will not be excluded from the PPS unless subjects have met other criteria excluding them from the PPS
(c) Not compliant with the study drug
(d) Out of window efficacy assessment at study Week 8 visit

Alternate criteria for exclusion from the Per Protocol Analysis Set were also applied to accommodate unforeseen events that occurred during the conduct of the study.

Analyses on Per Protocol Analysis Set will be of supportive purpose and limited to primary endpoint (i.e., “product Estimand”).

### Safety Analysis Set (SAF)

The Safety Analysis Set includes all enrolled subjects who received at least one dose of study treatment (MLS-101 or placebo). In analyses performed on the Safety Analysis Set, subjects will be analyzed according to their actual treatment received.

### PK/PD Analysis Set (PKPD)

PK/PD Analysis Set includes all subjects in the SAF who have sufficient data available for the analysis of pharmacokinetic and pharmacodynamic measurements. In the analyses based on PKPD, subjects will be analyzed according to the actual treatment received.

### Definition of Baseline

Baseline is defined as the last available observed value of the parameter of interest prior to the first administration of the investigational medicinal product (IMP) for the double-blinded treatment period.

For AOBP measurements and any other clinical or laboratory variable for which there are replicate evaluations at the screening and baseline visits, baseline is defined as the mean of the last two non-missing values prior to first administration of the IMP for the double-blinded treatment period.

Change from baseline is calculated as: post-baseline result−baseline result. Percentage change from baseline is calculated as: (change from baseline/baseline result)×100%.

### Summary of Results

There was a dose-response relationship across the QD dose range, with doses of 50 mg and 100 mg QD associated with a mean reduction in AOBP measured systolic BP of −11 to −13 mmHg (per-protocol, placebo-corrected, analysis of Part-1 100 mg cohort=−10.3 mmHg, Full-analysis set combining Part-1 and Part-2 (interim)=−9.9 mmHg, N=58 active).

Once daily dosing was as effective as twice daily dosing, with results in the two BID cohorts no better than those in the 50 mg and 100 mg QD cohorts.

In a pooled analysis of 50 mg QD, 100 mg QD, 12.5 mg BID and 25 mg BID cohorts (N=103) 25% of subjects demonstrated a change in BPsys >−25 mmHg and 41% a change >−15 mmHg.

Using automated office blook pressure (AOBP), there was little difference in treatment response between individuals in Part 1 and available results from Part 2, suggesting PRA does not appear to be a strong determinant of response (serum and urine aldosterone were also uninformative).

24 h ambulatory blood pressure measurements demonstrated overnight reduction of systolic BP of −11.5+/−2.9 mmHg in the 100 mg QD cohort, with an associated increase in nigh-time “BP-dipping”, consistent with persistent nocturnal benefit after morning dosing,

The pooled (Part 1 and Part 2) 100 mg QD safety set (n=60) showed good safety and tolerability, with no effects on serum cortisol, few episodes of mild or moderate hyperkalemia and no episodes of severe hyperkalemia.

### Automated Office Blood Pressure Results

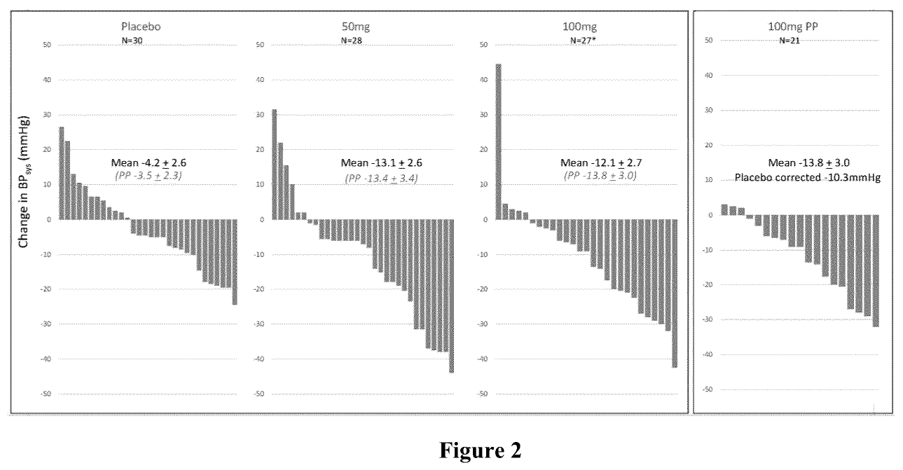

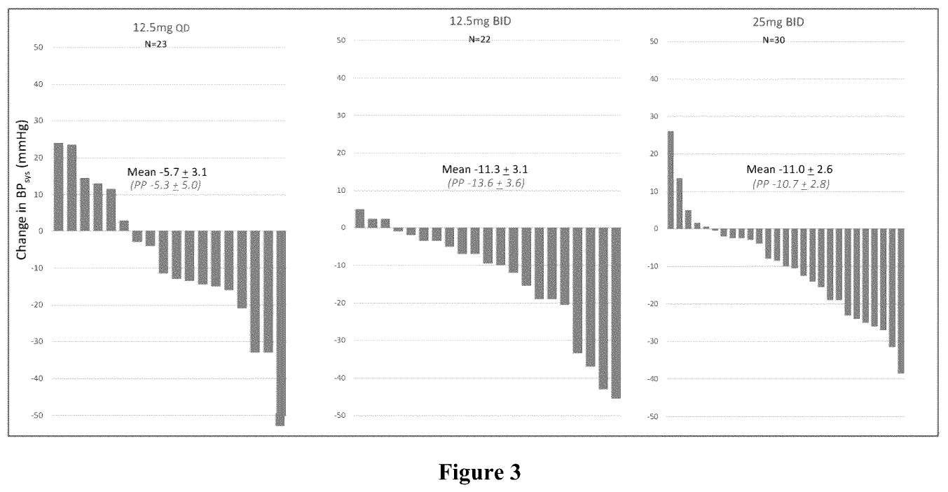

A full analysis and safety set (FAS) analysis was conducted using all subjects with week 8 measurement. A per-protocol (PP) analysis was also conducted using all subjects completing treatment through the eighth week visit. Waterfall plots showing the AOBP change in systolic blood pressure at week 8 from the FAS analysis of placebo, 50 mg QD and 100 mg QD groups and the PP analysis of the 100 mg group is provided in FIG. 2. Waterfall plots showing the AOBP change in systolic blood pressure at week 8 from the FAS analysis of 12.5 mg QD, 12.5 mg BID, and 25 mg BID groups is provided in FIG. 3. A modeled mean and per-protocol observed mean are also shown for each group.

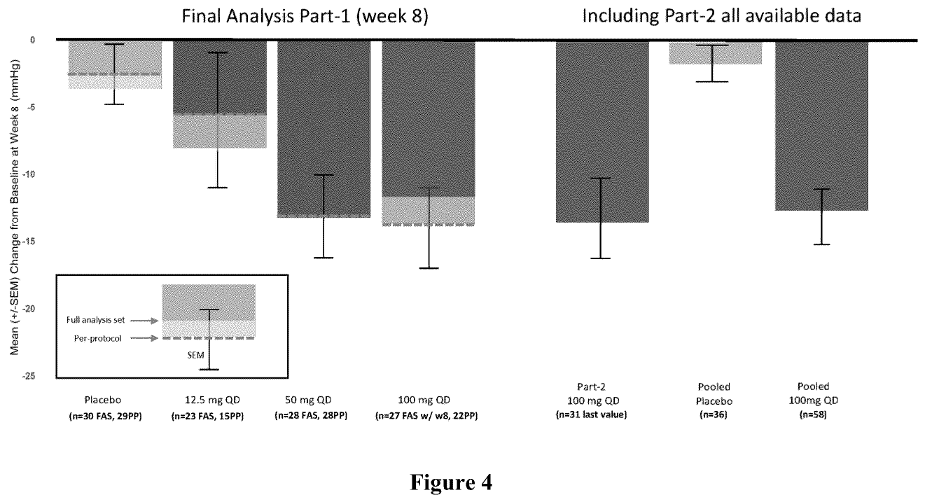

Mean change in systolic blood pressure from baseline is shown in FIG. 4. The figure provides a final analysis including both full analysis set (FAS, all evaluable subjects receiving at least one dose of Compound A HBr) and per-protocol (PP, only those receiving ≥75% of study drug with week 8 visit). Part 2 data shows the interim average of last visit week 5-6.

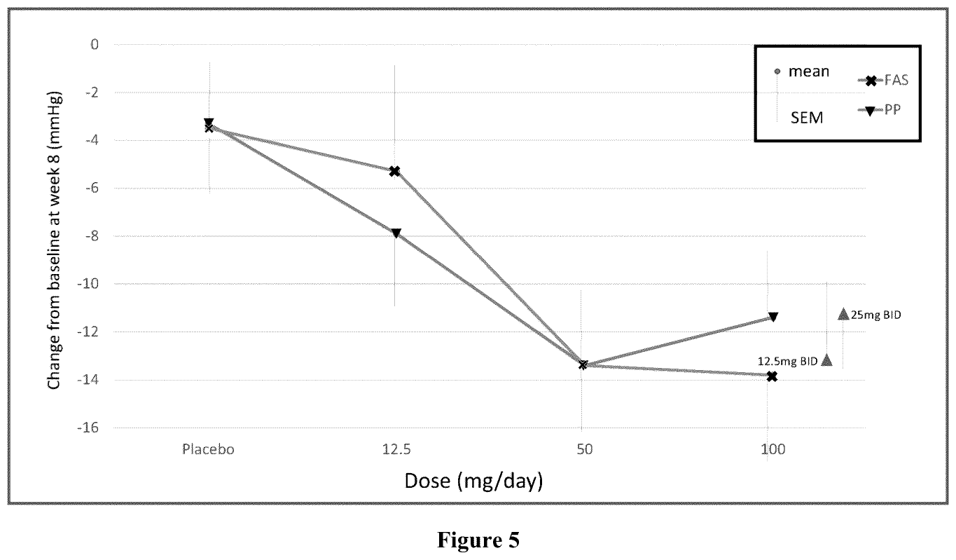

A Compound A HBr dose-response was observed based on analysis of systolic AOBP change from baseline among the QD regimens. FIG. 5 shows the mean observed automated office blood-pressure change from baseline at week 8 for QD dosing regimens. BID per-protocol cohorts are shown on the far right of this graph.

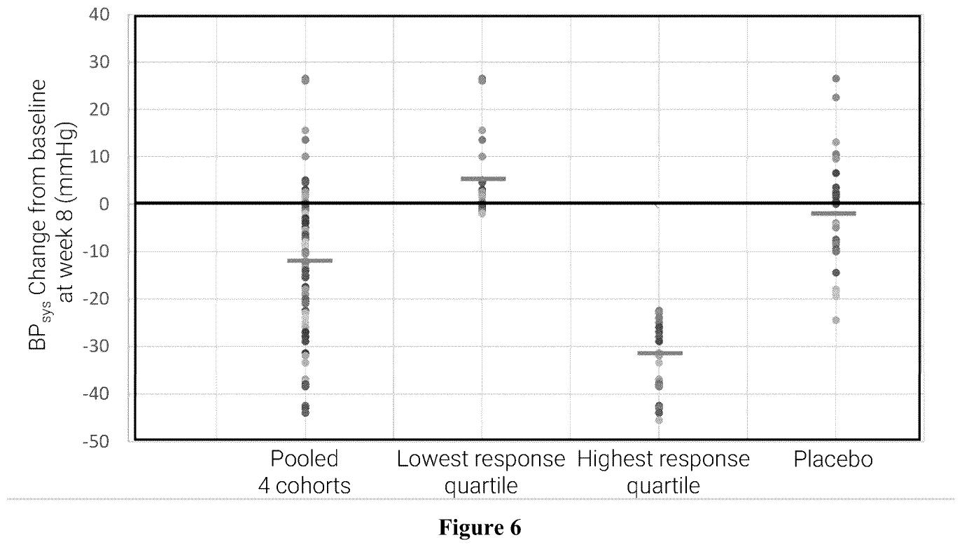

An analysis was conducted in which the change in systolic blood pressure from baseline at week 8 were pooled for the 50 mg QD, 100 mg QD, 12.5 mg BID, and 25 mg BID cohorts and then separated into quartiles based on degree of systolic blood pressure response. FIG. 6 is a graph showing the systolic blood pressure change from baseline at week 8 for the pooled cohorts, the lowest response quartile, the highest response quartile, and placebo. 25% of subjects achieved >−23 mmHg fall in systolic blood pressure, with mean reduction of −33.4+1.5 mmgHg. 41% of subjects achieved a ≥15 mmHg fall in systolic blood pressure.

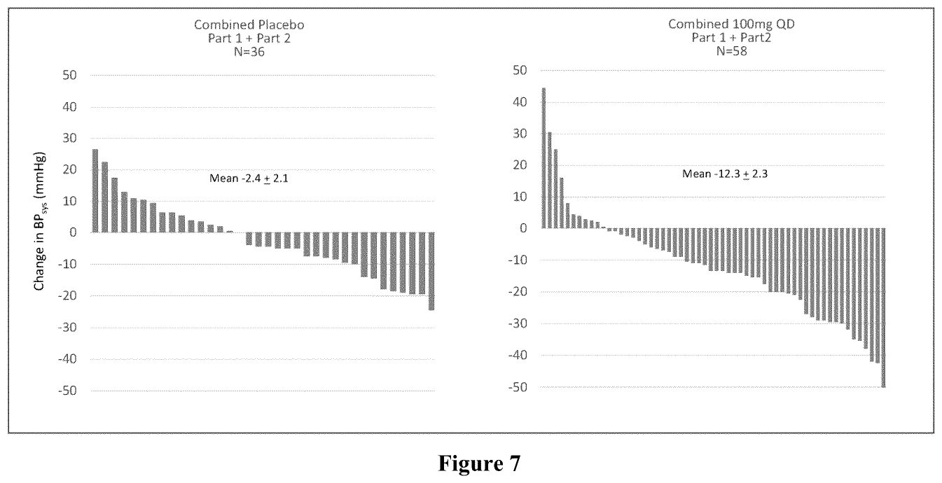

FIG. 7 is a waterfall plot showing change in systolic blood pressure from placebo and 100 mg QD groups pooled from both Parts 1 and 2. Part 2 data from interim snapshot with all subjects randomized and average last visit week 5-6, minimum week 2.

### Analysis of Factors Affecting Change in Blood Pressure

An analysis was conducted to identify factors affecting the degree of blood pressure reduction in hypertensive subjects. As summarized in the table below, it was discovered that Compound A HBr is particularly effective at reducing the blood pressure of individuals with a body mass index (BMI) of greater than 30. This effect was observed across dosing cohorts. A BMI of above 30 is considered to be in the obesity range. Similarly, a waist-hip ratio of greater than 0.90 in males and 0.85 in females is considered to be in the obesity range. Thus, it is inferred that Compound A HBr is particularly effective at reducing the blood pressure of males with a waist-hip ratio of greater than 0.90 and females with a waist-hip ratio of greater than 0.85.

Further, as summarized in the table below, it was discovered that Compound A HBr is particularly effective at reducing the blood pressure of individuals who were receiving a thiazide diuretic as one of their background hypertension medications. This effect was observed across dosing cohorts.

All values determined using least squares analysis of modeled means using all available information. **Difference in effect between 2 and 3+ background due to an imbalance in placebo response.

### Ambulatory Blood Pressure (ABPM) Results

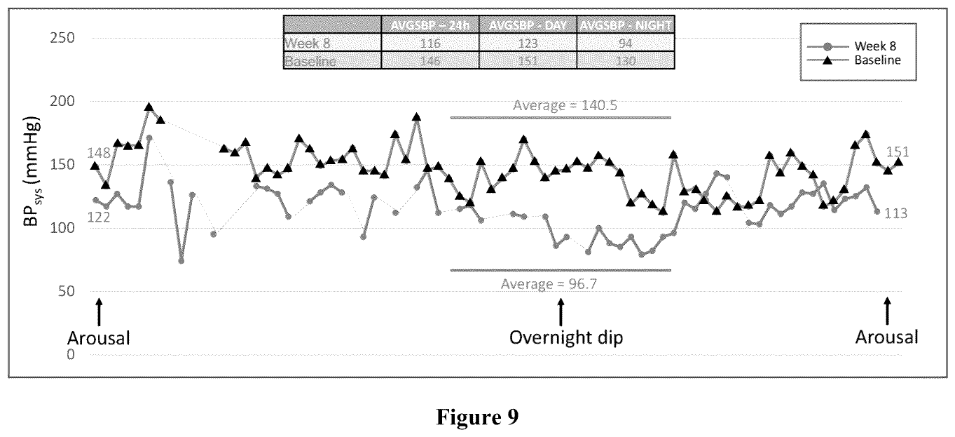

A graph showing an example of ambulatory 24-hour blood pressure monitoring is providing in FIG. 9. The graph shows the 24-hour ambulatory blood pressure (systolic) of a subject receiving Compound A HBr 100 mg QD versus baseline, showing an average 24-hour blood pressure reduction and restoration of normal nocturnal dipping pattern.

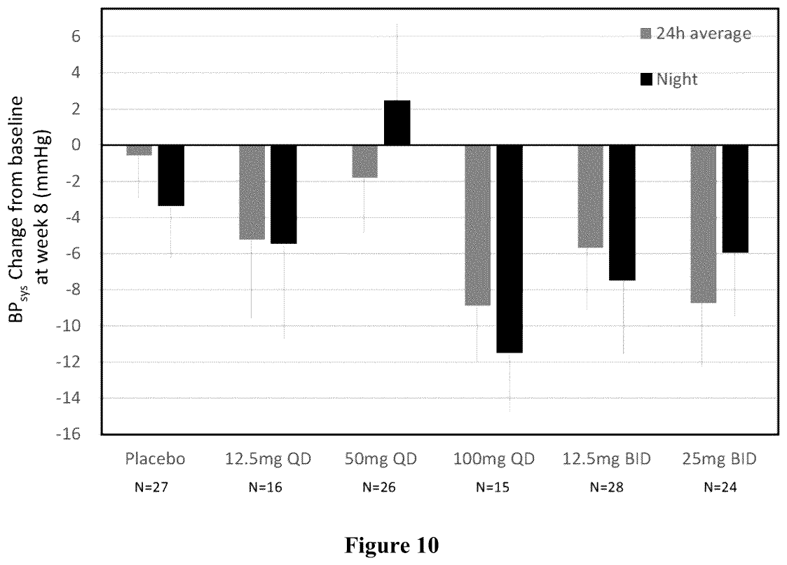

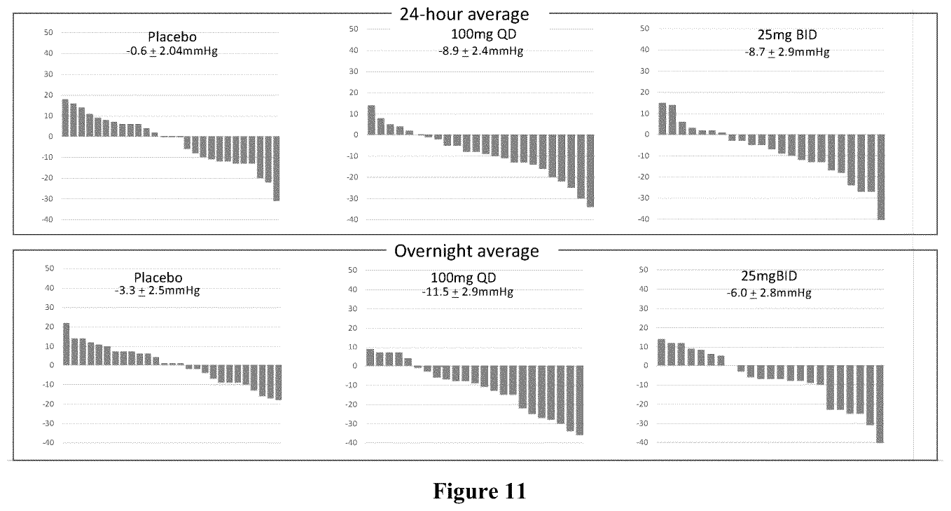

A graph showing the change in systolic blood pressure at week 8 relative to baseline as measured using the ABPM full analysis set is provided in FIG. 10. Waterfall plots showing the 24-hour average and overnight average ABPM change at 8 weeks relative to baseline is provided in FIG. 11. 100 mg QD dose levels provide excellent 24-hour blood pressure reduction. Overnight blood pressure reduction from the 100 mg QD dose level appears to be superior to 25 mg BID.

As shown in the summary table below, the most consistent benefit across all metrics was observed in the 100 mg QD cohort.

Night-time dip defined as 100%× (24-hour ambulatory Daytime SBP−24-hour ambulatory monitoring Night-time SBP)/24-hour ambulatory monitoring Daytime

### Safety

No serious adverse events (SAEs) related to the study medication were observed in the trial. Adverse events requiring drug discontinuation or dose reduction, showing incidence in Part 1 versus incidence Part 2 is provided in Table 4 below.

TABLE 4

| | | Total | Total | Part 1 | Part 1 | Part 2 | 100 mg QD |
|---|---|---|---|---|---|---|---|
| | | Placebo | active | All active | 100 mg QD | 100 mg QD | Pooled |
| | N | 35 | 190 | 159 | 29 | 31 | 60 |
| Study | Hypotension | 0 | 3 (1.6%) | 2 (1.2%) | 1 (3.4%) | 1 (2.8%) | 2 (3.3%) |
| drug | Hyponatremia | 0 | 1 (0.5%) | 0 | 0 | 1 (3.4%) | 1 (1.7%) |
| discontinuation | Hyperkalemia | 0 | 6 (3.2%) | 6 (3.8%) | 2 (6.9%) | 0 | 2 (3.3%) |
| | Other possibly | 0 | 1 (0.5%) | 1 (0.6%) | 1 (3.4%) | 0 | 1 (1.7%) |
| | related | | | | | | |
| | Other unrelated | 0 | 12 (6.3%) | 11 (6.9%) | 3 (6.0%) | 1 (2.8%) | 4 (6.7%) |
| Dose | Hyperkalemia | 0 | 7 (4.1%) | 6 (3.8%) | 1 (3.4%) | 1 (3.2%) | 2 (3.3%) |
| titration | Hyponatremia | 0 | 1 (0.5%) | 0 | 0 | 1 (3.2%) | 1 (1.7%) |
| | Other possibly | 0 | 0 | 0 | 0 | 0 | 0 |
| | related | | | | | | |
| | Other unrelated | 1 (1.4%) | 1 (0.5%) | 1 (0.6%) | 0 | 0 | 0 |

### Serum Potassium

Change in group mean serum potassium (K+) is shown in the table below.

TABLE 5

| | Part 1 | Part 2 | Pooled |
|---|---|---|---|
| Mean (mMol/L) | +0.50 | +0.23 | +0.35 |
| SD | 0.96 | 0.51 | 0.75 |
| N | 26* | 31 | 57 |
| SEM | 0.19 | 0.09 | 0.10 |

Number of individuals in the 100 mg QD cohorts with verified or multiple increased serum potassium above normal range during treatment is shown in the table below.

TABLE 6

| Serum | 5.2-5.5 | | 5.6-6.0 | | 6.1-6.5 | >6.5 |
|---|---|---|---|---|---|---|
| K+ | mMol/L | | mMol/L | | mMol/L | mMol/L |
| Part 1 | 5 | (18.5%) | 3 | (10.7%) | 0 | 1* |
| Part 2 | 1 | (4%) | 0 | | 0 | 0 |
| Pooled | 6 | (10.3%) | 3 | (5.2%) | 0 | 0 |

*Measure was an isolated incident not verified by repeat measurement with study drug discontinuation (protocol deviation).

Change in Estimated Glomerular Filtration Rate (eGFR)

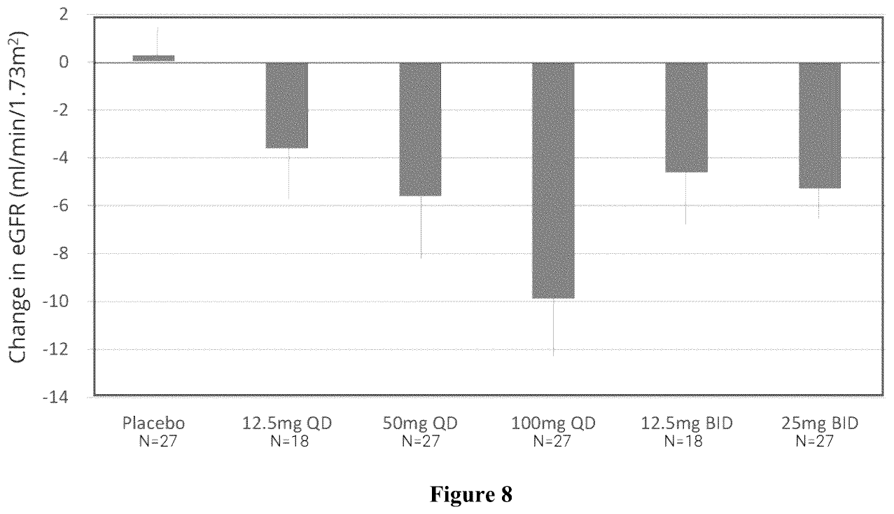

A dose-dependent and reversible reduction in eGFR was observed. This phenomenon has been reported with ACE/ARB and more recently SGLT2 inhibition due to reduced intra-glomerular pressure and felt to attenuate progression of hypertensive nephropathy. A graph showing the change in estimated glomerular filtration rate (eGFR) in different dosing cohorts is provided in FIG. 8.

### Example 2

A complete analysis of the study described in Example 1 was performed. This complete analysis confirmed the findings described above in Example 1 as follows.

### Summary of Results

Lorundrostat was effective at lowering BP in individuals with inadequately treated or treatment resistant hypertension.

There was a dose- and exposure-response relationship, with mean placebo-adjusted reduction in SBP of 9.58 mmHg and 7.81 mmHg seen at doses of 50 mg QD and 100 mg QD, respectively.

24-hour ABPM, central BP and nighttime SBP values confirmed the reduction in SBP seen with AOBP, most prominently in the 100 mg QD dose cohort.

The safety dataset lorundrostat was safe and well tolerated, with no effects on serum cortisol and an expected modest increase in serum potassium with relatively few episodes of clinically meaningful hyperkalemia.

The results demonstrate a clinically meaningful and statistically significant placebo-adjusted reduction in AOBP SBP of 9.58 mmHg (p=0.0114) and 7.81 mmHg (p=0.0422) in the 50 mg and 100 mg QD cohorts, respectively, in Part 1. The reduction in AOBP SBP observed in Part 2 for the 100 mg QD cohort was similar to that observed for the same dose in Part 1 (−11.5 mmHg, p=0.8426).

The reduction in AOBP SBP was validated and confirmed by comparable reductions in 24-hour ABPM SBP. The ABPM data further demonstrated the benefits of lorundrostat in reducing both central and nighttime SBP.

Sub-group analyses highlighted statistically and clinically significant reductions in AOBP SBP in subjects with a BMI >30 kg/m², those in the upper tertile of baseline AOBP SBP, and those taking thiazide diuretics.

Pharmacodynamic responses demonstrated dose-dependent reduction in serum aldosterone levels and a commensurate increase in plasma renin activity. 11-deoxycorticosterone levels were unaffected. Morning serum cortisol levels were modestly increased. No adrenocortical insufficiency occurred during the trial. Due to supply chain disruption, the ability to perform ACTH stimulation testing was limited during the first part of the trial. However, the majority of subjects in Part 2 dosed with 100 mg QD, the maximum dose tested, had ACTH-stimulation testing performed at Baseline and after 8 weeks of treatment. There were no individuals with abnormal ACTH-stimulation results.

Decreases in eGFR were observed in all treatment groups with greater decreases apparent for lorundrostat treatment groups compared with placebo.

Primary Efficacy Endpoint: Change from Baseline at Week 8 in Seated Automated Office-Measured Systolic Blood Pressure

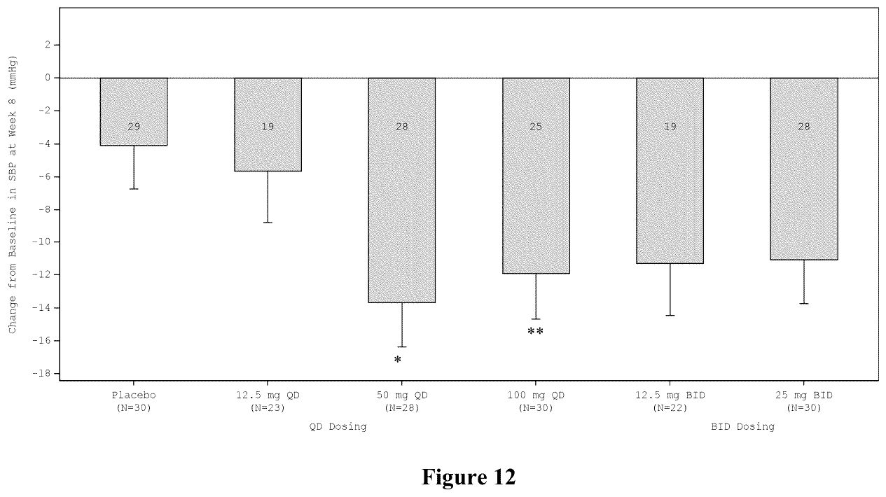

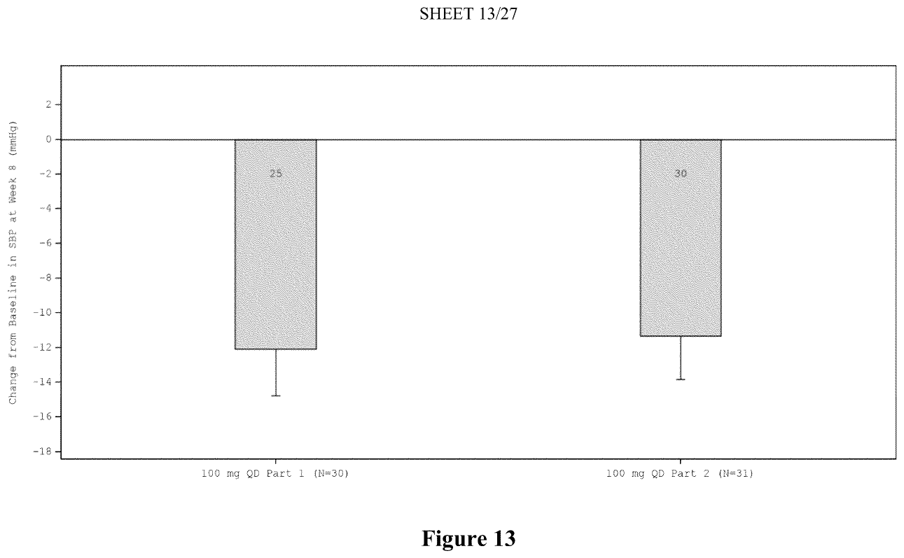

The results of a Mixed Model Repeated Measures (MMRM) analysis of the primary efficacy endpoint, change in seated AOBP SBP from Baseline to Week 8, is presented in FIG. 12 and Table 7 for Part 1 and in FIG. 13 and Table 8 for the 100 mg QD dose cohorts from Part 1 and Part 2.

At Week 8, a statistically significant reduction in modelled AOBP SBP from baseline was detected in the 50 mg QD cohort (least square mean [LSM] difference: −9.58 mmHg; p=0.0114) and 100 mg QD cohort (LSM difference: −7.81 mmHg; p=0.0422) in Part 1 (FIG. 12 and Table 7). While the 12.5 mg BID, 25 mg BID and 12.5 mg QD cohorts exhibited reductions in BP that were greater than placebo (placebo adjusted reductions of −7.2 mmHg, −6.97 mmHg, and −1.53 mmHg, respectively), none of these demonstrated statistical significance. The results were similar for the sensitivity analysis using the FAS, the analysis using the PPS, and the analysis using the additional estimand.

The change in SBP from baseline to Week 8 was similar for the 100 mg QD cohorts in Part 1 and Part 2 (LSM difference: 0.74 mmHg; p=0.8426) (FIG. 13 and Table 8). Further, no statistically significant appreciable difference in AOBP SBP change from baseline was seen between subjects who received lorundrostat 100 mg QD in Parts 1 and 2 combined who had a baseline PRA of <1 ng/mL/h versus those with a baseline PRA of ≥1 ng/mL/h (Table 9).

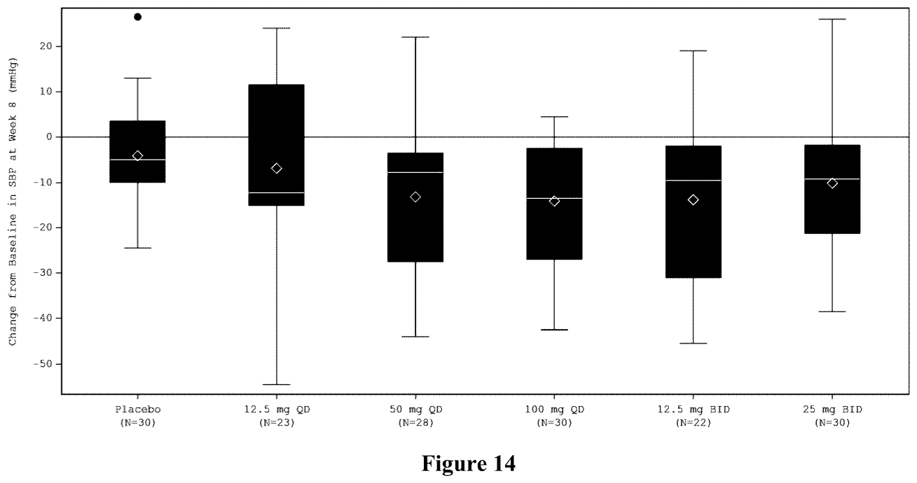

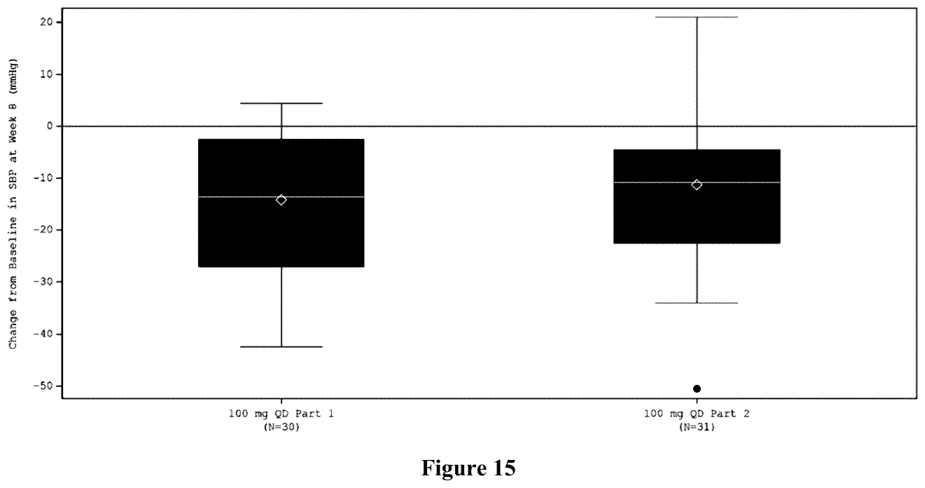

Boxplots of the mean changes in measured AOBP SBP from baseline to Week 8 are presented in FIG. 14 for Part 1 and in FIG. 15 for Part 2.

TABLE 7
Modelled (MMRM) Least Square Mean (SEM) Change from Baseline at Week 8 in Seated Automated
Office-Measured Systolic Blood Pressure (mmHg), Part 1 (Full Analysis Set)

| | Placebo | 12.5 mg BID | 25 mg BID | 12.5 mg QD | 50 mg QD | 100 mg QD |
|---|---|---|---|---|---|---|
| | (N = 30) | (N = 22) | (N = 30) | (N = 23) | (N = 28) | (N = 30) |
| Week 8 | | | | | | |
| n | 29 | 19 | 28 | 19 | 28 | 25 |
| LSM (SEM) | −4.10 (2.62) | −11.30 (3.15) | −11.07 (2.65) | −5.63 (3.16) | −13.69 (2.67) | −11.92 (2.77) |
| 90% CI | −8.44; 0.23 | −16.52; −6.08 | −15.46; −6.69 | −10.86; −0.41 | −18.11; −9.27 | −16.50; −7.33 |
| LSM Δ from | | −7.2 | −6.97 | −1.53 | −9.58 | −7.81 |
| Placebo | | | | | | |
| 90% CI | | −13.98; −0.41 | −13.13; −0.80 | −8.32; 5.27 | −15.77; −3.39 | −14.12; −1.50 |
| p-value | | 0.0813 | 0.0634 | 0.7103 | 0.0114 | 0.0422 |

Analysis used a MMRM approach with fixed effects for categorical terms for treatment, week, and treatment by week interaction, and baseline SBP as a fixed continuous covariate. No adjustment for multiplicity was implemented.

Abbreviations: BID, twice daily; CI, confidence interval; A, difference; LSM, least square mean; mg, milligrams; mmHg, millimeters of mercury; MMRM, mixed model repeated measures; QD, once daily; SBP, systolic blood pressure; SEM, standard error of the mean.

TABLE 8
Modelled (MMRM) Least Square Mean (SEM) Change from Baseline
at Week 8 in Seated Automated Office-Measured Systolic Blood
Pressure (mmHg), Parts 1 & 2 (Full Analysis Set)

| | 100 mg QD Part 1 | 100 mg QD Part 2 |
|---|---|---|
| Week 8 | (N = 30) | (N = 31) |
| n | 25 | 30 |
| LSM (SEM) | −12.08 (2.68) | −11.35 (2.48) |
| 90% CI | −16.56; −7.61 | −15.49; −7.20 |
| LSM Δ with | | 0.74 |
| Placebo | | |
| 90% CI | | −5.43; 6.90 |
| p-value | | 0.8426 |

Analysis used a MMRM approach with fixed effects for categorical terms for treatment, week, and treatment by week interaction, and baseline SBP as a fixed continuous covariate. No adjustment for multiplicity was implemented.

Abbreviations: CI, confidence interval; A, difference; LSM, least square mean; mg, milligrams; mmHg, millimeters of mercury; MMRM, mixed model repeated measures; QD, once daily; SBP, systolic blood pressure; SEM, standard error of the mean.

TABLE 9
Modelled (ANCOVA) Least Square Mean (SEM) Change from
Baseline at Week 4 and Week 8 in Seated Automated
Office-Measured Systolic Blood Pressure (mmHg) in
Subjects Randomized to 100 mg QD in Parts 1 & 2,
according to PRA level at Baseline (Full Analysis Set)

| | PRA Baseline < | PRA Baseline >= |
|---|---|---|
| FAS Randomized to 100 mg QD, | 1 ng/mL/h | 1 ng/mL/h |
| Part 1 and Part 2 Combined | (N = 27) | (N = 34) |
| Week 4 | | |
| n | 22 | 28 |
| LSM (SEM) | −15.42 (3.12) | −15.24 (2.75) |
| 90% CI | −20.66; −10.18 | −19.86; −10.62 |
| LSM Δ with PRA Baseline < 1 | | 0.18 |
| ng/mL/h | | |
| 90% CI | | −6.97; 7.33 |
| p-value | | 0.9663 |
| Week 8 | | |
| n | 22 | 33 |
| LSM (SEM) | −11.27 (3.13) | −13.34 (2.52) |
| 90% CI | −16.51; −6.02 | −17.55; −9.12 |
| LSM Δ with PRA Baseline < 1 | | −2.07 |
| ng/mL/h | | |
| 90% CI | | −9.04; 4.90 |
| p-value | | 0.6213 |

Analysis used an ANCOVA model with binary baseline PRA (<1 ng/mL/h versus ≥1 ng/mL/h) as a factor and baseline SBP as a covariate, in the subset of subjects randomized to receive 100 mg QD. The two ANCOVA models (Week 4 and Week 8) were run separately.

Abbreviations: ANCOVA, analysis of covariance; CI, confidence interval; Δ, difference; FAS, Full Analysis Set; h, hour; LSM, least square mean; mg, milligrams; mL, milliliter; mmHg, millimeters of mercury; ng, nanograms; PRA, plasma renin activity; QD, once daily; SBP, systolic blood pressure; SEM, standard error mean.

### Secondary Efficacy Endpoints

Change from Baseline at Week 8 in Seated Automated Office-Measured Diastolic Blood Pressure

At Week 4, statistically significant reductions in modelled AOBP DBP from baseline were detected across all cohorts in Part 1 except 12.5 mg QD. At Week 8, a statistically significant reduction in modelled AOBP DBP from baseline was detected in the 50 mg QD cohort only (LSM difference: −5.46 mmHg; p=0.0224), although all lorundrostat dose groups exhibited greater numerical declines than placebo (Table 10). The change in DBP from baseline to Week 8 was not statistically different between the 100 mg QD cohorts in Part 1 and Part 2 (LSM difference: 0.98 mmHg; p=0.6406) (Table 11).

TABLE 10
Modelled (MMRM) Least Square Mean (SEM) Change from Baseline at Weeks 4 and 8 in Seated Automated
Office-Measured Diastolic Blood Pressure (mmHg) by Dose, Part 1 (Full Analysis Set)

| | Placebo | 12.5 mg BID | 25 mg BID | 12.5 mg QD | 50 mg QD | 100 mg QD |
|---|---|---|---|---|---|---|
| | (N = 30) | (N = 22) | (N = 30) | (N = 23) | (N = 28) | (N = 30) |
| Week 4 | | | | | | |
| n | 28 | 19 | 27 | 21 | 28 | 25 |
| LSM (SEM) | −0.54 (1.40) | −5.02 (1.67) | −6.47 (1.41) | −2.50 (1.60) | −4.49 (1.41) | −6.96 (1.48) |
| 90% CI | −2.85; 1.78 | −7.78; −2.25 | −8.81; −4.14 | −5.15; 0.15 | −6.82; −2.15 | −9.40; −4.51 |
| LSM Δ with Placebo | | −4.48 | −5.94 | −1.96 | −3.95 | −6.42 |
| 90% CI | | −8.09; −0.87 | −9.24; −2.63 | −5.48; 1.55 | −7.22; −0.68 | −9.80; −3.04 |
| p-value | | 0.0418 | 0.0034 | 0.3569 | 0.0476 | 0.002 |
| Week 8 | | | | | | |
| n | 29 | 19 | 28 | 19 | 28 | 25 |
| LSM (SEM) | −1.63 (1.66) | −5.47 (2.02) | −4.12 (1.68) | −3.76 (2.02) | −7.09 (1.70) | −5.75 (1.77) |
| 90% CI | −4.38; 1.11 | −8.81; −2.14 | −6.91; −1.33 | −7.10; −0.42 | −9.90; −4.29 | −8.68; −2.82 |
| LSM Δ with Placebo | | −3.84 | −2.49 | −2.12 | −5.46 | −4.11 |
| 90% CI | | −8.16; 0.49 | −6.41; 1.44 | −6.45; 2.20 | −9.37; −1.54 | −8.14; −0.08 |
| p-value | | 0.1440 | 0.2964 | 0.4172 | 0.0224 | 0.0934 |

Analysis used a MMRM approach with fixed effects for categorical terms for treatment, week, and treatment by week interaction, and baseline DBP as a fixed continuous covariate. P-values ≤0.05 are bolded. Abbreviations: BID, twice daily; CI, confidence interval; Δ, difference; DBP, diastolic blood pressure; LSM, least square mean; mmHg, millimeters of mercury; QD, once daily; SEM, standard error mean.

TABLE 11
Modelled (MMRM) Least Square Mean (SEM) Change from Baseline
at Weeks 4 and 8 in Seated Automated Office-Measured Diastolic
Blood Pressure (mmHg) by Dose in Subjects Randomized to
100 mg QD, Parts 1 & 2 (Full Analysis Set)

| | 100 mg QD | 100 mg QD |
|---|---|---|
| FAS Randomized to 100 mg QD, | Part 1 | Part 2 |
| Part 1 and Part 2 Combined | (N = 30) | (N = 31) |
| Week 4 | | |
| n | 25 | 26 |
| LSM (SEM) | −5.88 (1.49) | −6.30 (1.44) |
| 90% CI | −8.38; −3.38 | −8.71; −3.89 |
| LSM Δ with 100 mg QD Part 1 | | −0.43 |
| 90% CI | | −3.90; 3.05 |
| p-value | | 0.8384 |
| Week 8 | | |
| n | 25 | 30 |
| LSM (SEM) | −4.57 (1.53) | −5.55 (1.42) |
| 90% CI | −7.13; −2.01 | −7.93; −3.18 |
| LSM Δ with 100 mg QD Part 1 | | −0.98 |
| 90% CI | | −4.47; 2.51 |
| p-value | | 0.6406 |

Analysis used a MMRM approach with fixed effects for categorical terms for treatment, week, and treatment by week interaction, and baseline DBP as a fixed continuous covariate. Abbreviations: CI, confidence interval; A, difference; DBP, diastolic blood pressure; FAS, Full Analysis Set; LSM, least square mean; mg, milligrams; mmHg, millimeters of mercury; MMRM, mixed model repeated measures; QD, once daily; SEM, standard error mean.

Change from Baseline to Week 8 in Seated Automated Office-Measured Blood Pressure Parameters

Mean SBP decreased over the first 4 to 5 weeks of treatment, including in the placebo arm (Table 12 and Table 13). The reductions in mean DBP (Table 14 and Table 15) were smaller than those observed for SBP, and the change from baseline in the placebo group was marginal. In general, the reductions in BP reached a plateau at around 4 weeks of treatment.

TABLE 12
Summary of Mean (SD) Change from Baseline in Seated Automated Office-Measured
Systolic Blood Pressure (mmHg) by Study Visit, Part 1 (Full Analysis Set)

| | Placebo | 12.5 mg BID | 25 mg BID | 12.5 mg QD | 50 mg QD | 100 mg QD |
|---|---|---|---|---|---|---|
| Visit | (N = 30) | (N = 22) | (N = 30) | (N = 23) | (N = 28) | (N = 30) |
| BL | 142.70 (10.24) | 144.57 (10.53) | 140.93 (9.61) | 147.09 (11.63) | 141.82 (10.86) | 143.80 (11.98) |
| Week 1 | 139.83 (10.84) | 135.68 (14.48) | 134.37 (14.17) | 141.43 (15.98) | 133.50 (12.23) | 144.41 (14.54) |
| CFB | −2.87 (10.24) | −8.89 (11.51) | −6.57 (10.78) | −5.65 (13.96) | −8.32 (14.27) | 0.06 (12.66) |
| Week 2 | 143.04 (11.33) | 132.59 (14.70) | 134.57 (17.75) | 143.73 (17.32) | 132.29 (10.55) | 139.04 (18.22) |
| CFB | −0.21 (11.65) | −11.98 (13.02) | −6.37 (13.43) | −1.86 (16.36) | −9.54 (13.78) | −5.64 (16.38) |
| Week 3 | 142.04 (13.94) | 137.70 (15.37) | 136.34 (19.78) | 142.80 (18.14) | 132.32 (13.45) | 136.56 (14.68) |
| CFB | −0.46 (15.13) | −5.80 (9.60) | −5.34 (18.35) | −4.15 (13.14) | −9.50 (16.67) | −9.20 (11.37) |
| Week 4 | 139.86 (12.10) | 132.95 (15.79) | 131.85 (13.59) | 143.00 (18.03) | 132.75 (11.85) | 129.76 (12.66) |
| CFB | −3.39 (12.65) | −10.71 (12.27) | −9.63 (12.34) | −3.95 (15.74) | −9.07 (15.77) | −14.60 (11.66) |
| Week 5 | 142.29 (14.76) | 131.44 (13.13) | 132.25 (16.30) | 144.21 (13.11) | 134.27 (15.39) | 130.96 (14.84) |
| CFB | −0.96 (12.68) | −12.53 (13.34) | −9.16 (15.72) | −3.53 (12.49) | −8.38 (15.19) | −13.40 (16.59) |
| Week 6 | 139.89 (11.93) | 129.79 (15.24) | 134.32 (13.96) | 139.00 (16.95) | 131.11 (12.49) | 138.73 (14.13) |
| CFB | −3.70 (13.86) | −14.00 (14.80) | −7.09 (11.66) | −8.64 (16.52) | −10.72 (14.86) | −6.62 (15.69) |
| Week 7 | 137.81 (13.53) | 131.89 (11.93) | 130.74 (15.42) | 136.56 (19.58) | 133.19 (16.39) | 134.32 (18.32) |
| CFB | −5.78 (13.89) | −11.76 (13.06) | −11.00 (13.60) | −11.08 (19.26) | −7.69 (18.91) | −11.04 (16.75) |
| Week 8 | 139.14 (14.15) | 131.89 (12.93) | 131.29 (16.59) | 140.78 (22.01) | 128.64 (13.69) | 131.28 (14.13) |
| CFB | −4.09 (11.71) | −13.79 (17.70) | −10.13 (14.02) | −6.86 (19.85) | −13.18 (17.17) | −14.08 (13.26) |

Baseline was defined as the average of the last two non-missing values prior to first dose of study treatment including unscheduled visits. Abbreviations: BID, twice daily; BL, baseline; CFB, change from baseline; mmHg, millimeters of mercury; QD, once daily; SD, standard deviation.

TABLE 13
Summary of Mean (SD) Change from Baseline in Seated
Automated Office-Measured Systolic Blood Pressure
(mmHg) by Study Visit, Part 2 (Full Analysis Set)

| | | |
|---|---|---|
| Visit | Placebo (N = 6) | 100 mg QD (N = 31) |

| | | | | |
|---|---|---|---|---|
| Baseline | 132.92 | (8.38) | 140.35 | (8.62) |
| Week 1 | 126.67 | (15.87) | 129.00 | (18.15) |
| CFB | −6.25 | (9.67) | −11.35 | (15.60) |
| Week 2 | 131.83 | (15.66) | 131.33 | (14.99) |
| CFB | −1.08 | (9.23) | −8.80 | (13.86) |
| Week 3 | 130.17 | (11.03) | 131.52 | (16.81) |
| CFB | −2.75 | (7.52) | −8.76 | (15.67) |
| Week 4 | 136.17 | (18.70) | 125.44 | (17.18) |
| CFB | 3.25 | (10.93) | −16.04 | (17.07) |
| Week 5 | 125.00 | (12.41) | 127.92 | (20.63) |
| CFB | −7.92 | (6.41) | −12.87 | (19.31) |
| Week 6 | 127.00 | (10.30) | 127.58 | (17.26) |
| CFB | −5.92 | (9.37) | −13.29 | (16.80) |
| Week 7 | 127.83 | (17.10) | 128.75 | (14.28) |
| CFB | −5.08 | (13.25) | −11.66 | (16.54) |
| Week 8 | 128.33 | (18.86) | 129.30 | (15.44) |
| CFB | −4.58 | (11.18) | −11.20 | (15.71) |

Baseline was defined as the average of the last two non-missing values prior to first dose of study treatment including unscheduled visits. Abbreviations: CFB, change from baseline; mmHg, millimeters of mercury; QD, once daily; SD, standard deviation.

TABLE 14
Summary of Mean (SD) Change from Baseline in Seated Automated Office-Measured
Diastolic Blood Pressure (mmHg) by Study Visit, Part 1 (Full Analysis Set)

| | Placebo | 12.5 mg BID | 25 mg BID | 12.5 mg QD | 50 mg QD | 100 mg QD |
|---|---|---|---|---|---|---|
| Visit | (N = 30) | (N = 22) | (N = 30) | (N = 23) | (N = 28) | (N = 30) |
| BL | 83.37 (8.76) | 81.55 (7.58) | 78.92 (7.95) | 82.39 (10.65) | 84.88 (6.37) | 78.87 (9.60) |
| Week 1 | 81.93 (8.93) | 75.59 (8.32) | 76.70 (9.28) | 85.26 (13.88) | 80.54 (7.58) | 79.70 (11.07) |
| CFB | −1.43 (7.41) | −5.95 (6.17) | −2.22 (5.68) | 2.87 (12.31) | −4.34 (8.86) | 0.98 (8.29) |
| Week 2 | 83.50 (10.75) | 74.23 (10.15) | 76.50 (10.74) | 81.23 (9.21) | 78.96 (8.27) | 76.36 (11.90) |
| CFB | 0.00 (7.89) | −7.32 (9.14) | −2.42 (8.03) | 0.68 (8.89) | −5.91 (7.38) | −2.14 (9.60) |
| Week 3 | 83.07 (10.25) | 79.95 (9.17) | 78.72 (15.35) | 81.80 (13.73) | 81.04 (9.15) | 72.84 (10.83) |
| CFB | −0.04 (10.23) | −0.93 (7.51) | −0.55 (14.99) | −1.20 (9.13) | −3.84 (8.83) | −5.48 (9.36) |
| Week 4 | 82.29 (9.68) | 75.79 (10.69) | 73.26 (7.79) | 81.20 (14.03) | 79.57 (8.36) | 71.60 (8.62) |
| CFB | −1.21 (6.86) | −4.18 (10.21) | −5.80 (7.01) | −1.80 (9.84) | −5.30 (8.33) | −6.36 (6.75) |
| Week 5 | 82.61 (9.05) | 73.50 (10.13) | 76.29 (9.91) | 83.74 (14.32) | 78.58 (7.53) | 72.60 (11.27) |
| CFB | −0.89 (7.34) | −7.00 (7.81) | −2.82 (8.82) | 0.82 (14.46) | −6.10 (7.22) | −5.36 (8.12) |
| Week 6 | 84.37 (13.27) | 74.95 (10.29) | 76.04 (10.57) | 79.06 (13.07) | 78.63 (8.17) | 74.77 (10.12) |
| CFB | 0.87 (13.94) | −6.47 (6.39) | −3.07 (8.82) | −4.17 (10.04) | −6.26 (8.48) | −3.33 (7.96) |
| Week 7 | 79.81 (8.39) | 74.21 (9.64) | 73.89 (10.76) | 77.11 (12.74) | 80.88 (11.41) | 73.48 (11.08) |
| CFB | −3.69 (6.06) | −5.76 (7.62) | −4.87 (8.84) | −6.11 (12.05) | −3.75 (11.03) | −4.12 (8.55) |
| Week 8 | 81.28 (9.42) | 75.21 (10.07) | 75.43 (13.06) | 81.44 (17.24) | 76.82 (9.82) | 73.00 (9.43) |
| CFB | −2.24 (8.37) | −5.84 (8.78) | −3.68 (11.71) | −1.78 (13.33) | −8.05 (9.13) | −5.46 (7.85) |

Baseline was defined as the average of the last two non-missing values prior to first dose of study treatment including unscheduled visits. Abbreviations: BID, twice daily; CFB, change from baseline; mmHg, millimeters of mercury; QD, once daily; SD, standard deviation.

TABLE 15
Summary of Mean (SD) Change from Baseline in Seated
Automated Office-Measured Diastolic Blood Pressure
(mmHg) by Study Visit, Part 2 (Full Analysis Set)

| | | |
|---|---|---|
| Visit | Placebo (N = 6) | 100 mg QD (N = 31) |

| | | | | |
|---|---|---|---|---|
| Baseline | 78.25 | (7.58) | 78.05 | (7.64) |
| Week 1 | 79.33 | (12.69) | 72.19 | (8.84) |
| CFB | 1.08 | (7.91) | −5.85 | (11.04) |
| Week 2 | 75.67 | (10.60) | 73.10 | (7.88) |
| CFB | −2.58 | (6.14) | −5.13 | (8.47) |
| Week 3 | 76.50 | (10.65) | 73.72 | (7.97) |
| CFB | −1.75 | (8.20) | −4.76 | (8.11) |
| Week 4 | 77.33 | (10.67) | 71.36 | (8.29) |
| CFB | −0.92 | (7.30) | −6.92 | (10.16) |
| Week 5 | 72.83 | (11.48) | 71.31 | (9.19) |
| CFB | −5.42 | (6.79) | −6.35 | (9.99) |
| Week 6 | 77.17 | (12.12) | 70.65 | (8.56) |
| CFB | −1.08 | (6.88) | −7.25 | (7.83) |
| Week 7 | 72.17 | (9.06) | 71.61 | (9.29) |
| CFB | −6.08 | (4.28) | −6.13 | (8.05) |
| Week 8 | 76.17 | (10.98) | 72.17 | (8.40) |
| CFB | −2.08 | (8.19) | −5.68 | (10.78) |

Baseline was defined as the average of the last two non-missing values prior to first dose of study treatment including unscheduled visits. Abbreviations: CFB, change from baseline; mmHg, millimeters of mercury; QD, once daily; SD, standard deviation.

Proportion of Subjects with Seated Automated Office-Measured Diastolic Blood Pressure ≤130/80 mmHg

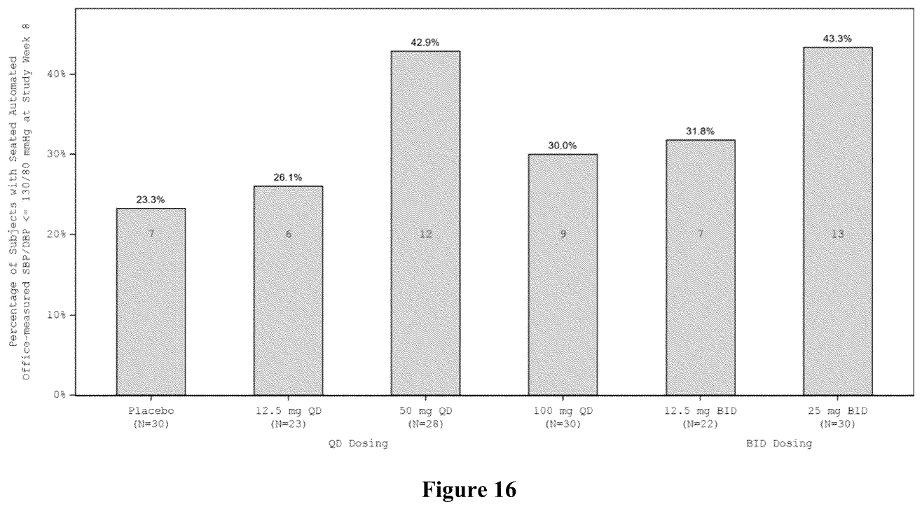

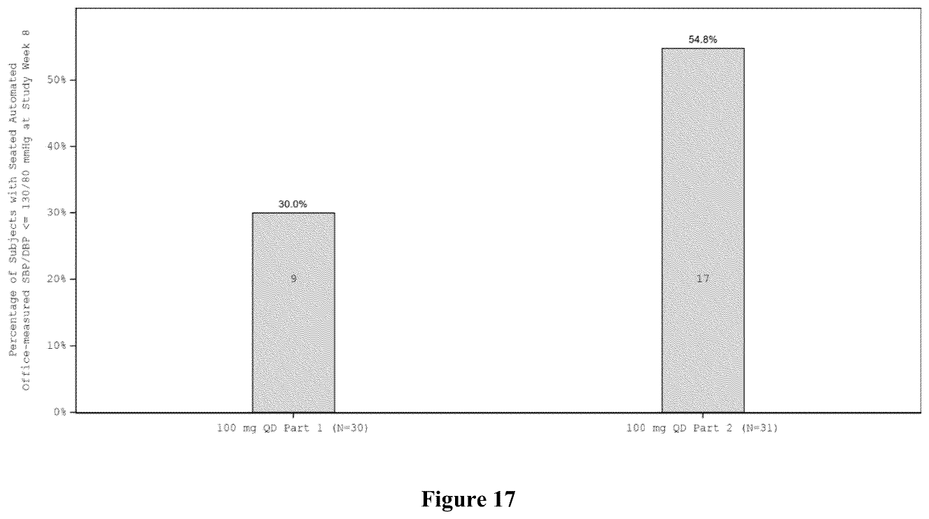

Overall, six subjects in Part 1 and three subjects in Part 2 had AOBP at or below the AHA cut-point for hypertension (130/80 mmHg) at baseline (Table 16 and Table 17). By Week 8 the proportion of subjects with AOBP ≤130/80 mmHg across all doses in Part 1 ranged from 23.3% for the Placebo cohort to 43.3% for the 25 mg BID cohort (FIG. 16). In Part 2, 54.8% of the 100 mg QD cohort achieved this treatment goal compared to 30% in the 100 mg QD cohort of Part 1 (FIG. 17).

TABLE 16
Proportion of Subjects with Seated Automated Office-Measured
SBP/DBP ≤130/80 mmHg at Week 8, Part 1 (Full Analysis Set)

| | | Placebo | 12.5 mg BID | 25 mg BID | 12.5 mg QD | 50 mg QD | 100 mg QD |
|---|---|---|---|---|---|---|---|
| Visit | | (N = 30) | (N = 22) | (N = 30) | (N = 23) | (N = 28) | (N = 30) |
| Baseline | SBP/DBP ≤130/ | | | | | | |
| | 80 mmHg | | | | | | |
| | Yes, n (%) | 1 (3.3) | 1 (4.5) | 3 (10.0) | 0 | 0 | 1 (3.3) |
| | No, n (%) | 29 (96.7) | 21 (95.5) | 27 (90.0) | 23 (100) | 28 (100) | 29 (96.7) |
| Week 8 | SBP/DBP ≤130/ | | | | | | |
| | 80 mmHg | | | | | | |
| | Yes, n (%) | 7 (23.3) | 7 (31.8) | 13 (43.3) | 6 (26.1) | 12 (42.9) | 9 (30.0) |
| | No, n (%) | 23 (76.7) | 15 (68.2) | 17 (56.7) | 17 (73.9) | 16 (57.1) | 21 (70.0) |

Baseline was defined as the average of the last two non-missing values prior to first dose of study treatment including unscheduled visits. Abbreviations: BID, twice daily; DBP, diastolic blood pressure; mmHg, millimeters of mercury; QD, once daily; SBP, systolic blood pressure.

TABLE 17
Proportion of Subjects with Seated Automated Office-Measured
SBP/DBP ≤ 130/80 mmHg at Week 8, Part 2 (Full Analysis Set)

| | | Placebo | 100 mg QD |
|---|---|---|---|
| Visit | | (N = 6) | (N = 31) |
| Baseline | SBP/DBP ≤ | | |
| | 130/80 mmHg | | |
| | Yes, n (%) | 1 (16.7) | 2 (6.5) |
| | No, n (%) | 5 (83.3) | 29 (93.5) |
| Week 8 | SBP/DBP ≤ | | |
| | 130/80 mmHg | | |
| | Yes, n (%) | 3 (50.0) | 17 (54.8) |
| | No, n (%) | 3 (50.0) | 14 (45.2) |

Baseline was defined as the average of the last two non-missing values prior to first dose of study treatment including unscheduled visits. Abbreviations: DBP, diastolic blood pressure; mmHg, millimeters of mercury; QD, once daily; SBP, systolic blood pressure.

Time to First Occurrence of Seated Automated Office-Measured SBP/DBP ≤130/80 mmHg

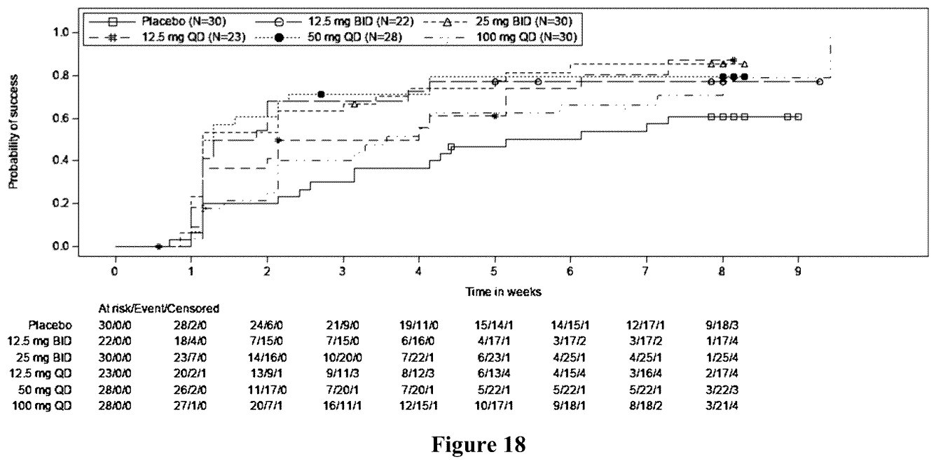

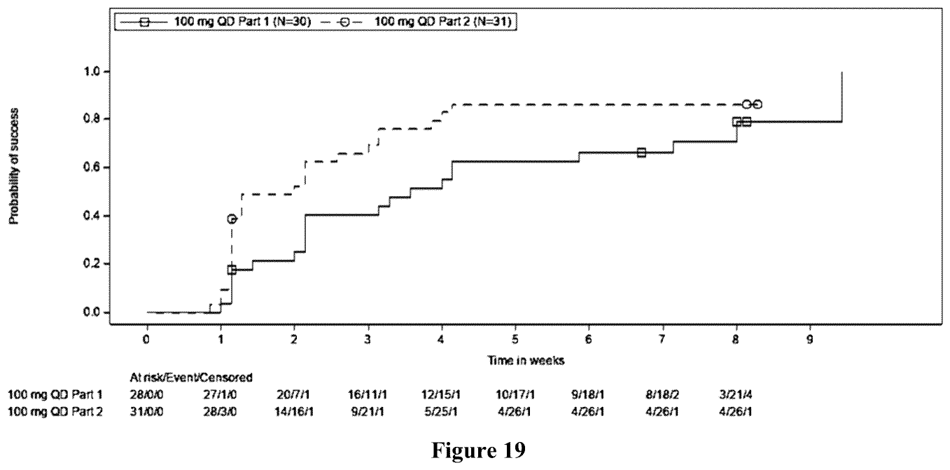

With the exception of subjects assigned to the Placebo group, the majority of subjects who achieved SBP/DBP of 130/80 mmHg did so between Weeks 2 and 4 of treatment in both Part 1 and Part 2 (FIG. 18 and FIG. 19).

Change from Baseline at End of Treatment in 24-Hour Ambulatory Blood Pressure Monitoring Parameters

The modelled changes in ABPM parameters (24-hour SBP, DBP, MAP, central SBP, central DBP and central MAP; daytime SBP, DBP and MAP; nighttime SBP and DBP) demonstrated a consistent AHT effect (Table 18 and Table 19). The results are compatible with those reported for AOBP parameters (SBP and DBP).

The changes from baseline to EoT in measured 24-hour, daytime and nighttime mean ABPM SBP, DBP and MAP are summarized in Table 20 and. The changes from baseline in mean 24-hour ABPM SBP and DBP at EoT were numerically smaller than those reported for AOBP which is an expected finding for the different modes of measurement.

TABLE 18
Modelled (ANCOVA) Least Square Mean Change from Baseline at End of Treatment
in 24-hour ABPM Parameters, Part 1 (Full Analysis Set; Post-hoc Analysis)

| 24-hr ABPM | | 12.5 mg BID | 25 mg BID | 12.5 mg QD | 50 mg QD | 100 mg QD |
|---|---|---|---|---|---|---|
| Parameter | Statistic | (N = 22) | (N = 30) | (N = 23) | (N = 28) | (N = 30) |
| 24-hr SBP | LSM Δ with Placebo | −4.76 | −8.04 | −1.5 | −3.85 | −5.33 |
| (mmHg) | p-value | 0.2181 | 0.0181 | 0.7043 | 0.2496 | 0.1256 |
| 24-hr DBP | LSM Δ with Placebo | −4.24 | −5.13 | −1.44 | −2.74 | −5.23 |
| (mmHg) | p-value | 0.0742 | 0.0139 | 0.5504 | 0.18 | 0.0141 |
| 24-hr Mean | LSM Δ with Placebo | −4.58 | −6.68 | −1.62 | −3.48 | −5.45 |
| MAP (mmHg) | p-value | 0.1268 | 0.0113 | 0.5951 | 0.1785 | 0.0422 |
| Daytime Mean | LSM Δ with Placebo | −4.96 | −8.76 | −2.14 | −6.85 | −4.12 |
| SBP (mmHg) | p-value | 0.2038 | 0.0109 | 0.5918 | 0.0424 | 0.2409 |
| Daytime Mean | LSM Δ with Placebo | −4.19 | −5.64 | −2.47 | −4.18 | −4.98 |
| DBP (mmHg) | p-value | 0.0828 | 0.0079 | 0.3155 | 0.0443 | 0.0216 |
| Nighttime Mean | LSM Δ with Placebo | −3.9 | −4.66 | −0.21 | 2.37 | −7.36 |
| SBP (mmHg) | p-value | 0.421 | 0.2823 | 0.9675 | 0.576 | 0.0931 |
| Nighttime Mean | LSM Δ with Placebo | −3.92 | −3.28 | −1.53 | 0.15 | −5.68 |
| DBP (mmHg) | p-value | 0.2022 | 0.2336 | 0.6328 | 0.9556 | 0.0427 |
| Mean Central | LSM Δ with Placebo | −7.19 | −10.19 | 1.92 | −5.46 | −7.79 |
| SBP (mmHg) | p-value | 0.1148 | 0.0069 | 0.6616 | 0.1481 | 0.0409 |
| Mean Central | LSM Δ with Placebo | −4.85 | −7.36 | −1.29 | −2.58 | −7.01 |
| DBP (mmHg) | p-value | 0.1123 | 0.0037 | 0.6623 | 0.3068 | 0.0063 |
| Mean Central | LSM Δ with Placebo | −6.08 | −8.87 | 0.06 | −4.11 | −7.77 |
| Mean BP (mmHg) | p-value | 0.1029 | 0.0041 | 0.9875 | 0.1826 | 0.0126 |

P-values in bold indicate p < 0.05.

TABLE 19
Modelled (ANCOVA) Least Square Mean Change from
Baseline at End of Treatment in 24-hour ABPM Parameters
in Subjects Randomized to 100 mg QD, Parts 1 &
2 (Full Analysis Set; Post-hoc Analysis)

| | | 100 mg QD |
|---|---|---|
| 24-hr ABPM | | Part 2 |
| Parameter | Statistic | (N = 31) |
| 24-hr Mean SBP | LSM Δ with 100 mg QD Part 1 | 4.13 |
| (mmHg) | p-value | 0.3145 |
| 24-hr Mean DBP | LSM Δ with 100 mg QD Part 1 | 4.26 |
| (mmHg) | p-value | 0.0984 |
| 24-hr Mean | LSM Δ with 100 mg QD Part 1 | 3.33 |
| AP (mmHg) | p-value | 0.2967 |
| Daytime Mean | LSM Δ with 100 mg QD Part 1 | 1.64 |
| SBP (mmHg) | p-value | 0.705 |
| Daytime Mean | LSM Δ with 100 mg QD Part 1 | 3.92 |
| DBP (mmHg) | p-value | 0.1326 |
| Nighttime Mean | LSM Δ with 100 mg QD Part 1 | 8.91 |
| SBP (mmHg) | p-value | 0.0422 |
| Nighttime Mean | LSM Δ with 100 mg QD Part 1 | 5.04 |
| DBP (mmHg) | p-value | 0.0755 |
| Mean Central | LSM Δ with 100 mg QD Part 1 | 4.68 |
| SBP (mmHg) | p-value | 0.2716 |
| Mean Central | LSM Δ with 100 mg QD Part 1 | 4.53 |
| DBP (mmHg) | p-value | 0.1211 |
| Mean Central | LSM Δ with 100 mg QD Part 1 | 3.98 |
| Mean BP (mmHg) | p-value | 0.2521 |

P-values in bold indicate p < 0.05.

TABLE 20
Measured Mean (SD) Change from Baseline to End of Treatment in 24-hour Ambulatory
Blood Pressure Monitoring Parameters, Part 1 (Full Analysis Set)

| | | Placebo | | 12.5 mg BID | | 25 mg BID | | 12.5 mg QD | | 50 mg QD | | 100 mg QD | |
|---|---|---|---|---|---|---|---|---|---|---|---|---|---|
| | Visit | (N = 30) | | (N = 22) | | (N = 30) | | (N = 23) | | (N = 28) | | (N = 30) | |
| 24-hr | BL | 138.30 | (12.62) | 139.86 | (11.90) | 138.97 | (15.30) | 142.82 | (8.61) | 133.14 | (16.48) | 143.41 | (14.56) |
| Mean SBP | EoT | 137.85 | (12.49) | 133.19 | (14.86) | 129.69 | (12.57) | 139.00 | (16.31) | 131.36 | (15.58) | 135.04 | (11.25) |
| (mmHg) | CFB | −0.81 | (10.73) | −5.69 | (12.90) | −8.73 | (15.67) | −5.20 | (16.55) | −1.79 | (15.82) | −8.88 | (11.90) |
| 24-hr | BL | 83.83 | (8.62) | 82.50 | (8.53) | 81.97 | (10.86) | 82.45 | (6.46) | 80.75 | (10.87) | 82.38 | (10.96) |
| Mean DBP | EoT | 82.62 | (9.70) | 77.63 | (11.03) | 76.50 | (10.59) | 79.93 | (7.33) | 77.75 | (9.33) | 75.92 | (8.42) |
| (mmHg) | CFB | −1.62 | (5.79) | −5.38 | (8.59) | −6.12 | (9.77) | −2.27 | (8.35) | −3.00 | (8.52) | −5.92 | (8.44) |
| 24-hr | BL | 111.07 | (9.76) | 111.18 | (9.36) | 110.47 | (12.25) | 112.64 | (5.46) | 106.95 | (13.03) | 112.90 | (11.32) |
| Mean | EoT | 110.23 | (10.11) | 105.41 | (12.62) | 103.10 | (10.92) | 109.47 | (10.81) | 104.55 | (11.74) | 105.48 | (8.26) |
| MAP | CFB | −1.21 | (7.66) | −5.53 | (10.63) | −7.42 | (12.49) | −3.73 | (12.21) | −2.39 | (11.80) | −7.40 | (10.04) |
| (mmHg) | | | | | | | | | | | | | |
| Daytime | BL | 140.43 | (11.30) | 140.64 | (12.35) | 142.40 | (16.83) | 144.55 | (8.48) | 136.86 | (14.62) | 145.66 | (14.64) |
| Mean SBP | EoT | 140.73 | (12.54) | 135.38 | (14.97) | 132.58 | (13.62) | 141.13 | (15.62) | 132.32 | (14.36) | 139.42 | (11.65) |
| (mmHg) | CFB | 0.42 | (12.23) | −4.06 | (14.48) | −9.08 | (17.41) | −4.80 | (15.10) | −4.54 | (13.59) | −7.08 | (12.60) |
| Daytime | BL | 85.77 | (9.53) | 83.59 | (8.59) | 84.70 | (11.74) | 84.36 | (6.49) | 83.61 | (9.34) | 84.03 | (11.04) |
| Mean DBP | EoT | 85.19 | (9.86) | 79.69 | (10.81) | 79.04 | (10.41) | 81.53 | (6.79) | 79.50 | (9.08) | 78.79 | (9.44) |
| (mmHg) | CFB | −0.92 | (6.75) | −4.25 | (9.64) | −6.23 | (10.43) | −2.60 | (8.43) | −4.11 | (6.81) | −4.96 | (8.74) |
| Nighttime | BL | 133.07 | (16.86) | 135.18 | (13.57) | 129.50 | (13.89) | 136.32 | (12.38) | 127.22 | (16.95) | 135.96 | (15.36) |
| Mean SBP | EoT | 130.58 | (14.77) | 126.81 | (16.69) | 123.92 | (12.17) | 132.00 | (18.11) | 129.54 | (21.68) | 124.50 | (14.93) |
| (mmHg) | CFB | −3.46 | (12.79) | −7.50 | (16.17) | −5.96 | (15.37) | −5.43 | (20.36) | 2.44 | (22.25) | −11.48 | (13.87) |
| Nighttime | BL | 78.77 | (8.07) | 77.95 | (9.66) | 74.10 | (9.84) | 78.05 | (7.91) | 73.85 | (11.94) | 77.21 | (13.13) |
| Mean DBP | EoT | 76.88 | (10.89) | 72.44 | (12.25) | 71.38 | (11.94) | 74.29 | (9.59) | 74.25 | (13.08) | 69.00 | (9.04) |
| (mmHg) | CFB | −2.46 | (9.51) | −6.00 | (10.17) | −4.13 | (10.86) | −3.21 | (9.55) | 0.00 | (12.34) | −6.57 | (9.97) |

TABLE 21
Measured Mean (SD) Change from Baseline to End of
Treatment in 24-hour Ambulatory Blood Pressure Monitoring
Parameters, Part 2 (Full Analysis Set)

| | Visit | Placebo (N = 6) | | 100 mg QD (N = 31) | |
|---|---|---|---|---|---|
| 24-hr Mean SBP | Baseline | 137.50 | (16.01) | 131.06 | (13.09) |
| (mmHg) | EoT | 128.83 | (15.66) | 132.59 | (16.72) |
| | CFB | −8.67 | (7.34) | 2.30 | (16.68) |
| 24-hr Mean DBP | Baseline | 80.00 | (9.53) | 76.42 | (8.92) |
| (mmHg) | EoT | 75.00 | (10.64) | 77.07 | (11.15) |
| | CFB | −5.00 | (3.10) | 1.33 | (10.97) |
| 24-hr Mean MAP | Baseline | 108.75 | (10.73) | 103.74 | (10.18) |
| (mmHg) | EoT | 101.92 | (12.28) | 104.83 | (12.95) |
| | CFB | −6.83 | (4.32) | 1.81 | (13.46) |
| Daytime Mean | Baseline | 139.33 | (16.19) | 133.71 | (12.56) |
| SBP (mmHg) | EoT | 130.33 | (14.50) | 135.22 | (16.83) |
| | CFB | −9.00 | (9.53) | 2.41 | (17.69) |
| Daytime Mean | Baseline | 81.00 | (10.00) | 78.52 | (8.81) |
| DBP (mmHg) | EoT | 76.83 | (10.91) | 79.41 | (11.01) |
| | CFB | −4.17 | (3.06) | 1.67 | (10.63) |
| Nighttime Mean | Baseline | 132.83 | (17.63) | 124.94 | (16.29) |
| SBP (mmHg) | EoT | 124.50 | (20.04) | 126.04 | (18.97) |
| | CFB | −8.33 | (8.07) | 1.52 | (16.44) |
| Nighttime Mean | Baseline | 76.83 | (10.34) | 71.16 | (10.77) |
| DBP (mmHg) | EoT | 69.83 | (11.20) | 71.85 | (12.27) |
| | CFB | −7.00 | (5.18) | 1.04 | (12.49) |

In many of the treatment cohorts, the proportion of subjects achieving a nighttime dip in ABPM SBP of ≥10%˜increased from baseline to the last post-baseline assessment (Table 22 and Table 23).

TABLE 22
Proportion of Subjects with Nighttime Dip in 24-hour Systolic
Ambulatory Blood Pressure, Part 1 (Full Analysis Set)

| | | Placebo | | 12.5 mg BID | | 25 mg BID | | 12.5 mg QD | | 50 mg QD | | 100 mg QD | |
|---|---|---|---|---|---|---|---|---|---|---|---|---|---|
| Visit | Category | (N = 30) | | (N = 22) | | (N = 30) | | (N = 23) | | (N = 28) | | (N = 30) | |
| Baseline | <10%, n (%) | 20 | (66.7) | 18 | (81.8) | 19 | (63.3) | 15 | (68.2) | 18 | (66.7) | 20 | (71.4) |
| | 10-20%, n (%) | 9 | (30.0) | 4 | (18.2) | 9 | (30.0) | 7 | (31.8) | 9 | (33.3) | 7 | (25.0) |
| | >20%, n (%) | 1 | (3.3) | 0 | | 2 | (6.7) | 0 | | 0 | | 1 | (3.6) |
| EoT | <10%, n (%) | 16 | (61.5) | 11 | (68.8) | 19 | (79.2) | 9 | (64.3) | 22 | (78.6) | 11 | (45.8) |
| | 10-20%, n (%) | 10 | (38.5) | 5 | (31.3) | 4 | (16.7) | 5 | (35.7) | 6 | (21.4) | 11 | (45.8) |

| | | | | | | | | |
|---|---|---|---|---|---|---|---|---|
| >20%, n (%) | 0 | 0 | 1 | (4.2) | 0 | 0 | 2 | (8.3) |

Nighttime Dip was defined as 100%×(24-hour ABPM Mean Daytime SBP−24-hour ABPM Mean Nighttime SBP)/24-hour ABPM Mean Daytime SBP.

TABLE 23
Proportion of Subjects with Nighttime Dip in 24-hour Systolic
Ambulatory Blood Pressure, Part 2 (Full Analysis Set)

| | | Placebo | | 100 mg QD | |
|---|---|---|---|---|---|
| Visit | Category | (N = 6) | | (N = 31) | |
| Baseline | <10%, n (%) | 4 | (66.7) | 19 | (61.3) |
| | 10-20%, n (%) | 2 | (33.3) | 11 | (35.5) |
| | >20%, n (%) | 0 | | 1 | (3.2) |
| Week 4 | <10%, n (%) | 6 | (100) | 18 | (72.0) |
| | 10-20%, n (%) | 0 | | 6 | (24.0) |
| | >20%, n (%) | 0 | | 1 | (4.0) |
| EoT | <10%, n (%) | 4 | (66.7) | 18 | (66.7) |
| | 10-20%, n (%) | 2 | (33.3) | 9 | (33.3) |

| | | |
|---|---|---|
| >20%, n (%) | 0 | 0 |

The Nighttime Dip was defined as 100%×(24-hour ABPM Mean Daytime SBP−24-hour ABPM Mean Nighttime SBP)/24-hour ABPM Mean Daytime SBP.

### Analysis of Factors Affecting Change in Blood Pressure

An analysis was conducted to identify factors affecting the degree of blood pressure reduction in hypertensive subjects. As shown in the tables below, it was discovered that Lorundrostat is particularly effective at reducing the blood pressure of individuals with a body mass index (BMI) of greater than 30. This effect was observed across dosing cohorts.

Further, as shown in the tables below, it was discovered that Lorundrostat is particularly effective at reducing the blood pressure of individuals who were receiving a thiazide diuretic as one of their background hypertension medications. This effect was observed across dosing cohorts.

There was no apparent consistent difference between mean change from baseline in AOBP SBP at Week 8 when summarized by sex (Male, Female) age (<65, 65-79, ≥80 years), race (Black or African American, Other), number of AHT medications at baseline (2, ≥3), or use of concomitant angiotensin converting enzyme inhibitors (ACEi) or ARBs in Part 1 (Table 24) or Part 2 (Table 25).

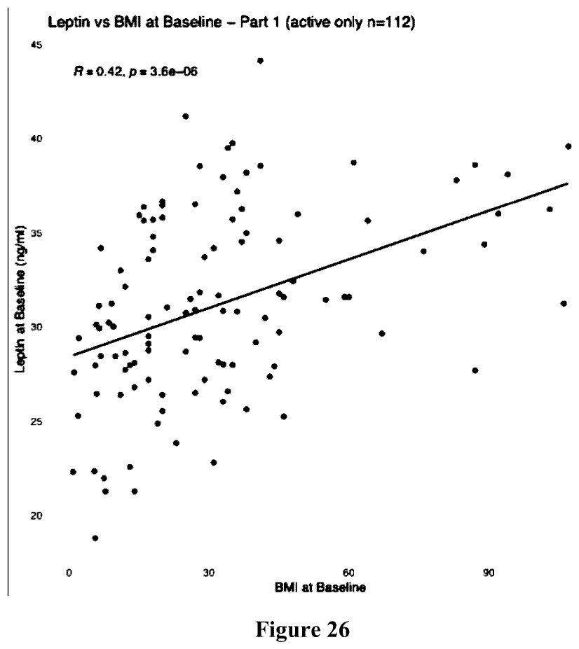

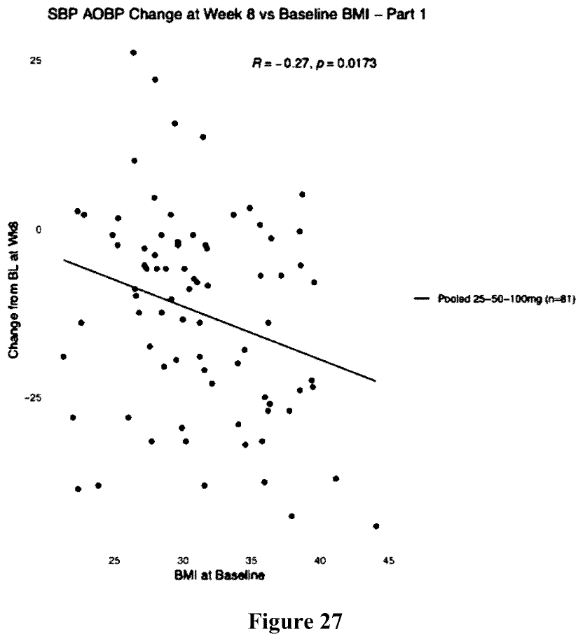

Using pooled data from low renin subjects (Part-1) from the lorundrostat 25 mg BID, 50 mg QD and 100 mg QD cohorts, the relationship between median baseline body mass index (BMI) and serum leptin (ng/dl) and the relationship between BMI at baseline and mean change in systolic BP (mmHg), measured by AOBP, at week 8 compared to baseline were analyzed (FIG. 26 and FIG. 27). Each of these cohorts demonstrated significant reduction in median serum aldosterone at week 4 compared to baseline. Treated as continuous variables, there was a strong relationship between increasing BMI and increasing serum leptin. Treated as continuous variables there was a strong relationship between BMI and reduction in BP. These two associations support the postulate that elevated serum leptin is associated with a larger reduction in BP. These data are consistent with the hypothesis that increased production of leptin in the setting of visceral obesity contributes to the increase in aldosterone-mediated hypertension. They further support the hypothesis that inhibition of aldosterone synthesis abrogates the stimulatory effect of leptin on aldosterone production.

TABLE 24
Mean (SD) Change from Baseline at Week 8 in Automated Office-Measured Systolic Blood Pressure, by Subgroup, Part 1 (Full Analysis Set)

| | Sub- | | Placebo | | 12.5 mg BID | | 25 mg BID | | 12.5 mg QD | | 50 mg QD | | 100 mg QD | |
|---|---|---|---|---|---|---|---|---|---|---|---|---|---|---|
| | group | Visit | (N = 30) | | (N = 22) | | (N = 30) | | (N = 23) | | (N = 28) | | (N = 30) | |
| Sex | Male | n | 13 | | 8 | | 11 | | 11 | | 13 | | 12 | |
| | | BL | 145.23 | (11.27) | 146.50 | (13.29) | 138.18 | (8.02) | 147.45 | (14.10) | 142.04 | (11.51) | 142.79 | (13.06) |
| | | Wk 8 | 142.92 | (10.00) | 134.40 | (9.42) | 128.82 | (16.45) | 145.56 | (26.10) | 132.08 | (15.20) | 129.60 | (11.45) |
| | | CFB | −3.79 | (8.91) | −17.50 | (15.84) | −9.36 | (13.49) | −4.50 | (26.71) | −9.96 | (15.67) | −15.45 | (15.48) |
| | Female | n | 17 | | 14 | | 19 | | 12 | | 15 | | 18 | |
| | | BL | 140.76 | (9.24) | 143.46 | (8.97) | 142.53 | (10.29) | 146.75 | (9.45) | 141.63 | (10.68) | 144.47 | (11.54) |
| | | Wk 8 | 136.47 | (16.22) | 131.00 | (14.17) | 132.88 | (16.99) | 136.00 | (17.23) | 125.67 | (11.97) | 132.40 | (15.95) |
| | | CFB | −4.29 | (13.61) | −12.46 | (18.69) | −10.62 | (14.75) | −9.22 | (10.54) | −15.97 | (18.44) | −13.17 | (12.05) |
| Age | <65 | n | 17 | | 6 | | 14 | | 10 | | 15 | | 9 | |
| (yr) | | BL | 140.68 | (9.44) | 139.08 | (9.02) | 140.39 | (6.49) | 149.30 | (15.40) | 140.73 | (11.21) | 147.00 | (12.65) |
| | | Wk 8 | 139.06 | (14.69) | 132.17 | (11.13) | 127.38 | (13.07) | 144.88 | (25.16) | 129.27 | (14.04) | 129.33 | (20.63) |
| | | CFB | −2.44 | (11.56) | −6.92 | (7.84) | −12.31 | (13.78) | −5.75 | (22.93) | −11.47 | (16.60) | −22.75 | (14.75) |
| | 65-79 | n | 12 | | 13 | | 15 | | 10 | | 13 | | 17 | |
| | | BL | 146.29 | (10.92) | 147.23 | (10.81) | 140.17 | (11.19) | 144.45 | (8.83) | 143.08 | (10.76) | 142.26 | (12.30) |
| | | Wk 8 | 138.58 | (14.47) | 135.73 | (11.26) | 133.86 | (19.37) | 135.63 | (15.23) | 127.92 | (13.82) | 133.73 | (12.53) |
| | | CFB | −7.71 | (11.04) | −12.77 | (19.08) | −7.82 | (14.88) | −8.50 | (14.04) | −15.15 | (18.27) | −9.53 | (11.49) |
| | ≥80 | n | 1 | | 3 | | 1 | | 3 | | 0 | | 4 | |
| | | BL | 134.00 | (NE) | 144.00 | (11.14) | 160.00 | (NE) | 148.50 | (2.78) | | | 143.13 | (10.41) |
| | | Wk 8 | 147.00 | (NE) | 110.00 | (1.41) | 146.00 | (NE) | 145.00 | (42.43) | | | 125.00 | (8.04) |
| | | CFB | 13.00 | (NE) | −40.00 | (4.24) | −14.00 | (NE) | −4.75 | (39.95) | | | −18.13 | (12.34) |
| Race | Black | n | 16 | | 7 | | 7 | | 11 | | 8 | | 15 | |
| | or | BL | 142.28 | (10.81) | 145.14 | (9.03) | 137.29 | (7.54) | 146.86 | (14.31) | 142.69 | (15.03) | 145.43 | (10.76) |
| | AA | Wk 8 | 137.00 | (16.33) | 137.71 | (13.50) | 124.00 | (15.68) | 147.50 | (22.06) | 130.38 | (14.12) | 129.92 | (16.18) |
| | | CFB | −5.28 | (13.58) | −7.43 | (20.17) | −13.29 | (15.91) | −1.44 | (16.04) | −12.31 | (18.13) | −17.13 | (11.55) |
| | Other | n | 14 | | 15 | | 23 | | 12 | | 20 | | 15 | |
| | | BL | 143.18 | (9.92) | 144.30 | (11.46) | 142.04 | (10.04) | 147.29 | (9.18) | 141.48 | (9.17) | 142.17 | (13.25) |
| | | Wk 8 | 141.77 | (10.95) | 128.50 | (11.84) | 133.71 | (16.53) | 135.40 | (21.55) | 127.95 | (13.83) | 132.54 | (12.47) |
| | | CFB | −2.62 | (9.21) | −17.50 | (15.80) | −9.07 | (13.59) | −11.20 | (22.29) | −13.53 | (17.25) | −11.27 | (14.54) |
| AHTs | 2 | n | 17 | | 7 | | 14 | | 14 | | 20 | | 14 | |
| | | BL | 142.38 | (11.91) | 144.00 | (14.58) | 143.54 | (9.04) | 150.32 | (13.04) | 139.88 | (9.41) | 140.39 | (9.10) |
| | | Wk 8 | 135.44 | (15.09) | 136.33 | (11.76) | 136.92 | (18.45) | 142.80 | (25.41) | 129.60 | (13.98) | 124.82 | (13.25) |
| | | CFB | −7.88 | (10.12) | −8.33 | (13.28) | −6.15 | (13.68) | −9.70 | (21.49) | −10.28 | (15.34) | −17.95 | (15.83) |
| | ≥3 | n | 13 | | 15 | | 16 | | 9 | | 8 | | 16 | |
| | | BL | 143.12 | (7.99) | 144.83 | (8.66) | 138.66 | (9.79) | 142.06 | (7.00) | 146.69 | (13.31) | 146.78 | (13.61) |
| | | Wk 8 | 143.69 | (11.90) | 129.85 | (13.37) | 126.40 | (13.57) | 138.25 | (18.26) | 126.25 | (13.56) | 136.36 | (13.05) |
| | | CFB | 0.58 | (12.21) | −16.31 | (19.35) | −13.57 | (13.83) | −3.31 | (18.37) | −20.44 | (20.36) | −11.04 | (10.45) |
| ACEi/ | Yes | n | 22 | | 17 | | 27 | | 18 | | 20 | | 23 | |
| ARB | | BL | 145.09 | (10.31) | 143.97 | (10.62) | 140.72 | (8.88) | 147.78 | (11.24) | 143.40 | (11.57) | 144.89 | (13.29) |
| | | Wk 8 | 140.64 | (14.76) | 132.00 | (12.59) | 132.20 | (15.32) | 141.50 | (23.12) | 127.65 | (14.07) | 134.90 | (13.12) |
| | | CFB | −4.45 | (12.63) | −12.47 | (17.09) | −9.04 | (14.04) | −6.39 | (16.84) | −15.75 | (17.46) | −11.68 | (13.23) |

| | | | | | | | |
|---|---|---|---|---|---|---|---|
| No | n | 8 | 5 | 3 | 5 | 8 | 7 |

| | | | | | | | | | | | | |
|---|---|---|---|---|---|---|---|---|---|---|---|---|
| BL | 136.13 | (6.93) | 146.60 | (11.17) | 142.83 | (17.55) | 144.60 | (14.05) | 137.88 | (8.19) | 140.21 | (5.08) |
| Wk 8 | 134.43 | (11.70) | 131.50 | (16.22) | 123.67 | (28.36) | 138.25 | (20.45) | 131.13 | (13.27) | 116.80 | (7.19) |
| CFB | −2.93 | (8.89) | −18.75 | (21.77) | −19.17 | (12.25) | −8.50 | (31.61) | −6.75 | (15.62) | −23.70 | (8.89) |

Abbreviations: AA, African American; ARB, angiotensin receptor blocker; ACEi, angiotensin converting enzyme inhibitor; AHT, antihypertensive; BID, twice daily; BL, baseline; CFB change from baseline; mg, milligrams; QD, once daily; SD, standard deviation; Wk 8, Week 8; yr, years.

TABLE 25
Mean (SD) Change from Baseline at Week 8 in Automated
Office-Measured Systolic Blood Pressure, by
Subgroup, Part 2 (Full Analysis Set)

| | Sub- | | Placebo | | 100 mg QD | |
|---|---|---|---|---|---|---|
| | group | Visit | (N = 6) | | (N = 31) | |
| Sex | Male | n | 2 | | 10 | |
| | | BL | 130.50 | (12.02) | 143.40 | (11.39) |
| | | Wk 8 | 122.50 | (28.99) | 132.10 | (10.89) |
| | | CFB | −8.00 | (16.97) | −11.30 | (17.09) |
| | Female | n | 4 | | 21 | |
| | | BL | 134.13 | (7.94) | 138.90 | (6.80) |
| | | Wk 8 | 131.25 | (16.70) | 127.90 | (17.36) |
| | | CFB | −2.88 | (10.04) | −11.15 | (15.43) |
| Age | <65 | n | 4 | | 8 | |
| (yr) | | BL | 135.88 | (8.09) | 133.94 | (5.73) |
| | | Wk 8 | 134.50 | (17.62) | 120.88 | (19.85) |
| | | CFB | −1.38 | (10.64) | −13.06 | (20.62) |
| | 65-79 | n | 2 | | 22 | |
| | | BL | 127.00 | (7.07) | 142.20 | (8.40) |
| | | Wk 8 | 116.00 | (19.80) | 132.81 | (12.82) |
| | | CFB | −11.00 | (12.73) | −9.69 | (13.82) |
| | ≥80 | n | 0 | | 1 | |
| | | BL | | | 151.00 | (NE) |
| | | Wk 8 | | | 123.00 | (NE) |
| | | CFB | | | −28.00 | (NE) |
| Race | Black | n | 2 | | 6 | |
| | or | BL | 136.25 | (13.08) | 141.67 | (7.70) |
| | AA | Wk 8 | 139.00 | (22.63) | 125.83 | (7.96) |
| | | CFB | 2.75 | (9.55) | −15.83 | (11.63) |
| | Other | n | 4 | | 25 | |
| | | BL | 131.25 | (6.99) | 140.04 | (8.94) |
| | | Wk 8 | 123.00 | (17.57) | 130.17 | (16.82) |
| | | CFB | −8.25 | (11.15) | −10.04 | (16.57) |
| AHTs | 2 | n | 4 | | 20 | |
| | | BL | 128.25 | (4.79) | 138.10 | (7.83) |
| | | Wk 8 | 118.00 | (11.92) | 127.84 | (16.77) |
| | | CFB | −10.25 | (8.66) | −10.37 | (15.86) |
| | ≥3 | n | 2 | | 11 | |
| | | BL | 142.25 | (4.60) | 144.45 | (8.81) |
| | | Wk 8 | 149.00 | (8.49) | 131.82 | (13.19) |
| | | CFB | 6.75 | (3.89) | −12.64 | (16.10) |
| ACEi/ | Yes | n | 5 | | 30 | |
| ARB | | BL | 134.10 | (8.79) | 140.53 | (8.71) |
| | | Wk 8 | 129.40 | (20.89) | 129.24 | (15.71) |
| | | CFB | −4.70 | (12.50) | −11.45 | (15.92) |

| | | | |
|---|---|---|---|
| No | n | 1 | 1 |

| | | | | |
|---|---|---|---|---|
| BL | 127.00 | (NE) | 135.00 | (NE) |
| Wk 8 | 123.00 | (NE) | 131.00 | (NE) |
| CFB | −4.00 | (NE) | −4.00 | (NE) |

Abbreviations: AA, African American; ARB, angiotensin receptor blocker; ACEi, angiotensin converting enzyme inhibitor; AHT, antihypertensive; BL, baseline; CFB change from baseline; mg, milligrams; NE, not evaluable; QD, once daily; SD, standard deviation; Wk 8, Week 8; yr, years.

There was an apparent increase in the AHT effect of lorundrostat with increasing BMI (Table 26) that was most apparent in the 100 mg QD dose cohorts in Part 1 (Table 26) and Part 2 (Table 27). Exploratory analysis using MMRM resulted in a placebo-adjusted reduction in AOBP SBP of −16.7 mmHg (p=0.0023) in the Part 1 50 mg QD cohort and −12.3 mmHg (p=0.0297) in the Part 1 100 mg QD cohort (data not shown).

TABLE 26
Mean (SD) Change from Baseline at Week 8 in Automated Office-Measured
Systolic Blood Pressure, by BMI at Baseline, Part 1 (Full Analysis Set)

| Sub- | | Placebo | | 12.5 mg BID | | 25 mg BID | | 12.5 mg QD | | 50 mg QD | | 100 mg QD | |
|---|---|---|---|---|---|---|---|---|---|---|---|---|---|
| group | Visit | (N = 30) | | (N = 22) | | (N = 30) | | (N = 23) | | (N = 28) | | (N = 30) | |
| <25 | n | 1 | | 2 | | 4 | | 3 | | 1 | | 5 | |
| kg/m² | BL | 164.00 | (NE) | 139.75 | (10.96) | 144.38 | (12.41) | 150.00 | (4.92) | 156.00 | (NE) | 144.90 | (18.74) |
| | Wk 8 | 159.00 | (NE) | 150.00 | (NE) | 126.25 | (18.52) | 137.00 | (NE) | 118.00 | (NE) | 146.00 | (18.33) |
| | CFB | −5.00 | (NE) | 2.50 | (NE) | −18.13 | (15.56) | −11.50 | (NE) | −38.00 | (NE) | −7.83 | (17.47) |
| 25-30 | n | 13 | | 4 | | 10 | | 8 | | 11 | | 10 | |
| kg/m² | BL | 146.19 | (11.88) | 149.38 | (10.16) | 137.00 | (8.59) | 141.06 | (6.90) | 139.09 | (10.46) | 139.95 | (8.19) |
| | Wk 8 | 138.62 | (14.65) | 125.50 | (14.89) | 134.11 | (15.72) | 134.14 | (18.96) | 136.64 | (14.70) | 129.67 | (10.05) |
| | CFB | −7.58 | (8.31) | −23.88 | (24.55) | −4.89 | (13.17) | −7.71 | (19.07) | −2.45 | (15.21) | −11.17 | (11.94) |
| >30 | n | 16 | | 16 | | 15 | | 12 | | 15 | | 14 | |
| kg/m² | BL | 138.53 | (5.52) | 143.97 | (10.82) | 142.07 | (9.48) | 150.38 | (13.97) | 143.40 | (11.05) | 146.79 | (11.75) |
| | Wk 8 | 138.27 | (13.68) | 132.43 | (11.93) | 130.93 | (17.43) | 145.80 | (24.73) | 124.13 | (10.98) | 129.00 | (15.28) |
| | CFB | −1.00 | (13.91) | −12.07 | (15.48) | −11.13 | (13.83) | −5.80 | (22.31) | −19.27 | (15.13) | −18.50 | (13.40) |

Abbreviations: BID, twice daily; BL, baseline; CFB, change from baseline; NE, not evaluable; QD, once daily; SD, standard deviation; Wk 8, Week 8.

TABLE 27
Mean (SD) Change from Baseline at Week 8 in Automated
Office-Measured Systolic Blood Pressure, by BMI
at Baseline, Part 2 (Full Analysis Set)

| Sub- | | Placebo | | 100 mg QD | |
|---|---|---|---|---|---|
| group | Visit | (N = 6) | | (N = 31) | |
| <25 | n | 1 | | 2 | |
| kg/m² | BL | 132.00 | (NE) | 136.50 | (2.12) |
| | Wk 8 | 117.00 | (NE) | 130.00 | (1.41) |
| | CFB | −15.00 | (NE) | −6.50 | (3.54) |
| 25-30 | n | 0 | | 15 | |
| kg/m² | BL | | | 141.40 | (7.98) |
| | Wk 8 | | | 126.80 | (17.49) |
| | CFB | | | −14.60 | (15.76) |
| >30 | n | 5 | | 14 | |
| kg/m² | BL | 133.10 | (9.36) | 139.79 | (9.93) |
| | Wk 8 | 130.60 | (20.16) | 132.08 | (14.23) |
| | CFB | −2.50 | (11.12) | −8.00 | (16.62) |

Abbreviations: BL, baseline; CFB, change from baseline; NE, not evaluable; QD, once daily; SD, standard deviation; Wk 8, Week 8.

There was an apparent increase in the AHT effect of lorundrostat with increasing baseline AOBP SBP in both Part 1 and Part 2 (Table 28 and Table 29, respectively). It should be noted that a greater AHT effect with increasing baseline AOBP SBP was also observable in the Part 1 placebo cohort and only the 50 mg QD cohort in Part 1 demonstrated a statistically significant placebo-adjusted reduction in AOBP SBP from baseline at Week 8 (−19.9 mmHg, p=0.0113).

TABLE 28
Mean (SD) Change from Baseline at Week 8 in Automated Office-Measured Systolic Blood
Pressure, by Systolic Blood Pressure Tertile at Baseline, Part 1 (Full Analysis Set)

| Sub- | | Placebo | | 12.5 mg BID | | 25 mg BID | | 12.5 mg QD | | 50 mg QD | | 100 mg QD | |
|---|---|---|---|---|---|---|---|---|---|---|---|---|---|
| group | Visit | (N = 30) | | (N = 22) | | (N = 30) | | (N = 23) | | (N = 28) | | (N = 30) | |
| Low | n | 10 | | 5 | | 11 | | 8 | | 12 | | 10 | |
| | BL | 133.75 | (3.16) | 132.80 | (4.72) | 131.91 | (4.93) | 135.13 | (1.71) | 132.83 | (3.43) | 132.15 | (6.41) |
| | Wk 8 | 131.11 | (13.06) | 132.25 | (10.34) | 120.70 | (13.06) | 131.17 | (12.77) | 130.25 | (12.04) | 129.86 | (9.26) |
| | CFB | −3.33 | (12.89) | −0.75 | (5.48) | −12.50 | (13.48) | −3.75 | (13.24) | −2.58 | (12.54) | −4.71 | (8.58) |
| Middle | n | 12 | | 10 | | 11 | | 4 | | 8 | | 9 | |
| | BL | 141.08 | (2.64) | 141.90 | (3.14) | 140.86 | (2.56) | 144.88 | (0.85) | 140.63 | (1.73) | 140.94 | (2.14) |
| | Wk 8 | 139.50 | (13.81) | 131.75 | (14.00) | 135.45 | (15.29) | 143.33 | (22.23) | 127.63 | (13.35) | 124.56 | (13.30) |
| | CFB | −1.58 | (13.38) | −10.56 | (16.34) | −5.41 | (14.64) | −1.17 | (21.80) | −13.00 | (14.05) | −16.39 | (13.97) |
| High | n | 8 | | 7 | | 8 | | 11 | | 8 | | 11 | |
| | BL | 156.31 | (8.92) | 156.79 | (7.57) | 153.44 | (5.64) | 156.59 | (8.92) | 156.50 | (6.88) | 156.73 | (6.96) |
| | Wk 8 | 147.63 | (11.86) | 131.86 | (14.81) | 139.86 | (16.99) | 146.33 | (26.41) | 127.25 | (17.59) | 139.11 | (15.36) |
| | CFB | −8.69 | (6.44) | −24.93 | (18.46) | −14.14 | (13.63) | −10.83 | (23.91) | −29.25 | (14.31) | −19.06 | (12.91) |

Abbreviations: BID, twice daily; BL, baseline; CFB, change from baseline; QD, once daily; SD, standard deviation; Wk 8, Week 8.

TABLE 29
Mean (SD) Change from Baseline at Week 8 in Automated Office-
measured Systolic Blood Pressure, by Systolic Blood Pressure
Tertile at Baseline, Part 2 (Full Analysis Set)

| Sub- | | Placebo | 100 mg QD |
|---|---|---|---|
| group | Visit | (N = 6) | (N = 31) |
| Low | n | 4 | 10 |

| | | | | |
|---|---|---|---|---|
| BL | 128.25 | (4.79) | 132.10 | (3.28) |
| Wk 8 | 118.00 | (11.92) | 124.11 | (19.94) |
| CFB | −10.25 | (8.66) | −7.56 | (19.91) |

| | | | |
|---|---|---|---|
| Middle | n | 1 | 10 |

| | | | | |
|---|---|---|---|---|
| BL | 139.00 | (NE) | 138.65 | (1.20) |
| Wk 8 | 143.00 | (NE) | 130.60 | (16.91) |
| CFB | 4.00 | (NE) | −8.05 | (16.54) |

| | | | |
|---|---|---|---|
| High | n | 1 | 11 |

| | | | | |
|---|---|---|---|---|
| BL | 145.50 | (NE) | 149.41 | (7.12) |
| Wk 8 | 155.00 | (NE) | 132.36 | (8.91) |
| CFB | 9.50 | (NE) | −17.05 | (9.63) |

Abbreviations: BL, baseline; CFB, change from baseline; NE, not evaluable; QD, once daily; SD, standard deviation; Wk 8, Week 8.

There also appeared to be a greater reduction in AOBP SBP from baseline in subjects with concomitant use of thiazide diuretics in Part 1 (Table 30), but this was not as apparent in Part 2 (Table 31). In Part 1, statistically significant placebo-adjusted reductions in AOBP SBP at Week 8 were achieved in the 12.5 mg BID and 50 mg QD cohorts with mean changes of −10.7 mmHg (p=0.047) and −12.9 mmHg (p=0.0108), respectively.

TABLE 30
Mean (SD) Change from Baseline at Week 8 in Automated Office-Measured Systolic Blood
Pressure, by Thiazide Diuretic Use at Baseline [Yes/No], Part 1 (Full Analysis Set)

| Sub- | | Placebo | | 12.5 mg BID | | 25 mg BID | | 12.5 mg QD | | 50 mg QD | | 100 mg QD | |
|---|---|---|---|---|---|---|---|---|---|---|---|---|---|
| group | Visit | (N = 30) | | (N = 22) | | (N = 30) | | (N = 23) | | (N = 28) | | (N = 30) | |
| Yes | n | 16 | | 13 | | 18 | | 12 | | 16 | | 17 | |
| | BL | 142.84 | (8.18) | 145.69 | (9.02) | 141.64 | (9.28) | 141.46 | (8.43) | 144.94 | (12.46) | 144.88 | (10.09) |
| | Wk 8 | 139.81 | (13.46) | 127.58 | (11.07) | 130.53 | (17.84) | 132.44 | (14.02) | 127.63 | (14.08) | 128.27 | (16.26) |
| | CFB | −3.03 | (12.33) | −19.25 | (16.90) | −10.65 | (14.39) | −7.89 | (14.31) | −17.31 | (17.91) | −17.37 | (14.74) |
| No | n | 14 | | 9 | | 12 | | 11 | | 12 | | 13 | |
| | BL | 142.54 | (12.51) | 142.94 | (12.82) | 139.88 | (10.41) | 153.23 | (11.82) | 137.67 | (6.71) | 142.38 | (14.38) |
| | Wk 8 | 138.31 | (15.47) | 139.29 | (13.28) | 132.45 | (15.21) | 149.11 | (26.02) | 130.00 | (13.65) | 135.80 | (9.10) |
| | CFB | −5.38 | (11.25) | −4.43 | (15.93) | −9.32 | (14.08) | −5.83 | (25.10) | −7.67 | (15.11) | −9.15 | (9.27) |

Abbreviations: BID, twice daily; BL, baseline; CFB, change from baseline; QD, once daily; Wk 8, Week 8.

TABLE 31
Mean (SD) Change from Baseline at Week 8 in Automated Office-
Measured Systolic Blood Pressure, by Thiazide Diuretic Use
at Baseline [Yes/No], Part 2 (Full Analysis Set)

| Sub- | | Placebo | 100 mg QD |
|---|---|---|---|
| group | Visit | (N = 6) | (N = 31) |
| Yes | n | 5 | 19 |

| | | | | |
|---|---|---|---|---|
| BL | 135.10 | (7.21) | 141.89 | (8.86) |
| Wk 8 | 133.60 | (15.39) | 131.72 | (11.71) |
| CFB | −1.50 | (9.22) | −10.50 | (13.87) |

| | | | |
|---|---|---|---|
| No | n | 1 | 12 |

| | | | | |
|---|---|---|---|---|
| BL | 122.00 | (NE) | 137.92 | (7.98) |
| Wk 8 | 102.00 | (NE) | 125.67 | (19.81) |
| CFB | −20.00 | (NE) | −12.25 | (18.73) |

Abbreviations: BL, baseline; CFB, change from baseline; NE, not evaluable; QD, once daily; Wk 8, Week 8.

There was no apparent consistent difference between mean change from baseline in AOBP DBP at Week 8 when summarized by sex (male, female), age (<65, 65-79, ≥80 years), race (Black or African American, Other), BMI (<25, 25-30, >30 kg/m²), seated AOBP DBP at baseline, number of AHT medications at baseline (2, ≥3), or use of concomitant ACEi or ARBs in Part 1 (Table 32) or Part 2 (Table 33).

TABLE 32
Mean (SD) Change from Baseline at Week 8 in Automated Office-Measured Diastolic Blood Pressure, by Subgroup, Part 1 (Full Analysis Set)

| | Sub- | | Placebo | | 12.5 mg BID | | 25 mg BID | | 12.5 mg QD | | 50 mg QD | | 100 mg QD | |
|---|---|---|---|---|---|---|---|---|---|---|---|---|---|---|
| Category | group | Visit | (N = 30) | | (N = 22) | | (N = 30) | | (N = 23) | | (N = 28) | | (N = 30) | |
| Sex | Male | n | 13 | | 8 | | 11 | | 11 | | 13 | | 12 | |
| | | BL | 85.42 | (6.00) | 85.44 | (10.67) | 78.45 | (9.55) | 86.27 | (12.87) | 84.88 | (8.36) | 84.29 | (7.81) |
| | | Wk 8 | 84.92 | (8.22) | 78.40 | (9.63) | 72.82 | (9.34) | 87.78 | (21.70) | 78.15 | (6.52) | 76.20 | (6.81) |
| | | CFB | −1.04 | (9.31) | −7.50 | (5.61) | −5.64 | (7.70) | 1.28 | (17.06) | −6.73 | (7.65) | −8.15 | (8.28) |
| | Female | n | 17 | | 14 | | 19 | | 12 | | 15 | | 18 | |
| | | BL | 81.79 | (10.30) | 79.32 | (4.13) | 79.18 | (7.14) | 78.83 | (6.89) | 84.87 | (4.28) | 75.25 | (9.11) |
| | | Wk 8 | 78.71 | (9.59) | 74.07 | (10.32) | 77.12 | (15.02) | 75.11 | (8.40) | 75.67 | (12.10) | 70.87 | (10.51) |
| | | CFB | −3.09 | (7.82) | −5.25 | (9.78) | −2.41 | (13.79) | −4.83 | (8.08) | −9.20 | (10.37) | −3.67 | (7.27) |
| Age | <65 | n | 17 | | 6 | | 14 | | 10 | | 15 | | 9 | |
| (yr) | | BL | 87.06 | (7.30) | 84.83 | (5.48) | 83.29 | (5.64) | 84.85 | (14.74) | 87.87 | (4.25) | 86.39 | (5.68) |
| | | Wk 8 | 84.56 | (7.83) | 79.17 | (9.58) | 76.54 | (7.57) | 82.63 | (20.67) | 80.53 | (9.93) | 78.33 | (9.69) |
| | | CFB | −3.00 | (7.04) | −5.67 | (5.02) | −6.69 | (8.06) | −2.19 | (9.98) | −7.33 | (9.30) | −8.67 | (10.16) |
| | 65-79 | n | 12 | | 13 | | 15 | | 10 | | 13 | | 17 | |
| | | BL | 78.13 | (8.59) | 81.81 | (7.33) | 74.73 | (7.97) | 81.50 | (5.98) | 81.42 | (6.79) | 76.85 | (8.73) |
| | | Wk 8 | 76.92 | (10.29) | 76.18 | (7.87) | 74.71 | (17.26) | 77.25 | (5.52) | 72.54 | (8.04) | 72.80 | (7.89) |
| | | CFB | −1.21 | (10.43) | −3.18 | (7.91) | −0.46 | (14.22) | −5.06 | (7.49) | −8.88 | (9.23) | −4.37 | (7.01) |
| | ≥80 | n | 1 | | 3 | | 1 | | 3 | | 0 | | 4 | |
| | | BL | 83.50 | (NE) | 73.83 | (9.17) | 80.50 | (NE) | 77.17 | (5.77) | | | 70.50 | (10.56) |
| | | Wk 8 | 81.00 | (NE) | 58.00 | (7.07) | 71.00 | (NE) | 93.50 | (37.48) | | | 65.75 | (11.73) |
| | | CFB | −2.50 | (NE) | −21.00 | (9.90) | −9.50 | (NE) | 13.00 | (37.48) | | | −4.75 | (8.03) |
| Race | Black or | n | 16 | | 7 | | 7 | | 11 | | 8 | | 15 | |
| | AA | BL | 85.81 | (8.77) | 78.93 | (5.16) | 83.21 | (7.11) | 84.95 | (13.98) | 88.13 | (3.96) | 79.20 | (9.98) |
| | | Wk 8 | 83.69 | (10.16) | 79.14 | (8.49) | 75.43 | (10.05) | 85.00 | (19.41) | 83.25 | (8.88) | 71.92 | (10.83) |
| | | CFB | −2.13 | (9.04) | 0.21 | (7.03) | −7.79 | (9.03) | −0.69 | (7.56) | −4.88 | (8.84) | −7.38 | (7.65) |
| | Other | n | 14 | | 15 | | 23 | | 12 | | 20 | | 15 | |
| | | BL | 80.57 | (8.17) | 82.77 | (8.36) | 77.61 | (7.87) | 80.04 | (6.05) | 83.58 | (6.76) | 78.53 | (9.54) |
| | | Wk 8 | 78.31 | (7.80) | 72.92 | (10.54) | 75.43 | (14.14) | 78.60 | (15.76) | 74.25 | (9.14) | 74.00 | (8.26) |
| | | CFB | −2.38 | (7.82) | −9.38 | (7.89) | −2.31 | (12.36) | −2.65 | (17.00) | −9.33 | (9.15) | −3.69 | (7.91) |
| BMI | <25 | n | 1 | | 2 | | 4 | | 3 | | 1 | | 5 | |
| (kg/m²) | | BL | 89.00 | (NE) | 74.75 | (15.91) | 77.50 | (3.56) | 74.17 | (3.51) | 85.50 | (NE) | 81.00 | (10.52) |
| | | Wk 8 | 83.00 | (NE) | 92.00 | (NE) | 69.25 | (9.50) | 73.00 | (NE) | 61.00 | (NE) | 78.33 | (19.14) |
| | | CFB | −6.00 | (NE) | 6.00 | (NE) | −8.25 | (7.63) | −1.50 | (NE) | −24.50 | (NE) | −0.67 | (7.01) |
| | 25-30 | n | 13 | | 4 | | 10 | | 8 | | 11 | | 10 | |
| | | BL | 80.38 | (8.06) | 79.75 | (3.88) | 76.85 | (10.34) | 82.00 | (5.76) | 84.77 | (7.28) | 79.70 | (11.37) |
| | | Wk 8 | 77.77 | (10.02) | 71.75 | (9.07) | 78.89 | (20.58) | 76.00 | (6.22) | 80.27 | (6.29) | 72.67 | (9.33) |
| | | CFB | −2.62 | (9.24) | −8.00 | (7.56) | 1.11 | (16.18) | −4.64 | (7.81) | −4.50 | (8.75) | −6.50 | (7.61) |
| | >30 | n | 16 | | 16 | | 15 | | 12 | | 15 | | 14 | |
| | | BL | 85.44 | (9.06) | 82.84 | (7.20) | 80.33 | (7.20) | 84.71 | (13.38) | 85.73 | (5.35) | 77.96 | (8.69) |
| | | Wk 8 | 84.20 | (8.41) | 75.00 | (9.80) | 75.00 | (6.97) | 86.10 | (21.92) | 76.07 | (11.02) | 71.42 | (7.10) |
| | | CFB | −1.67 | (8.09) | −6.07 | (9.01) | −5.33 | (8.88) | 0.20 | (16.85) | −9.67 | (8.59) | −6.88 | (7.81) |
| Seated | Low | n | 10 | | 5 | | 11 | | 8 | | 12 | | 10 | |
| SBP Tertile | | BL | 81.30 | (7.04) | 77.30 | (8.82) | 77.64 | (9.33) | 83.31 | (4.92) | 83.50 | (8.31) | 75.95 | (9.36) |
| | | Wk 8 | 78.11 | (9.06) | 78.50 | (9.88) | 72.40 | (9.55) | 78.50 | (7.48) | 81.08 | (9.65) | 73.86 | (11.01) |
| | | CFB | −3.44 | (7.02) | −2.25 | (7.56) | −6.15 | (8.84) | −4.17 | (6.69) | −2.42 | (8.22) | −0.29 | (6.44) |
| | Middle | n | 12 | | 10 | | 11 | | 4 | | 8 | | 9 | |
| | | BL | 85.04 | (10.15) | 83.50 | (7.71) | 78.18 | (8.42) | 76.25 | (5.81) | 84.63 | (4.07) | 79.39 | (8.71) |
| | | Wk 8 | 84.67 | (8.33) | 73.88 | (9.48) | 79.09 | (17.89) | 79.00 | (5.57) | 74.00 | (10.06) | 73.22 | (5.97) |
| | | CFB | −0.38 | (7.76) | −6.69 | (10.60) | 0.91 | (15.57) | 0.83 | (5.62) | −10.63 | (7.25) | −6.17 | (6.64) |
| | High | n | 8 | | 7 | | 8 | | 11 | | 8 | | 11 | |
| | | BL | 83.44 | (9.03) | 81.79 | (6.23) | 81.69 | (4.91) | 83.95 | (14.28) | 87.19 | (4.67) | 81.09 | (10.64) |
| | | Wk 8 | 79.75 | (10.82) | 74.86 | (11.87) | 74.00 | (7.39) | 84.22 | (23.90) | 73.25 | (8.33) | 72.11 | (11.78) |
| | | CFB | −3.69 | (10.91) | −6.93 | (7.79) | −7.36 | (5.62) | −1.06 | (18.28) | −13.94 | (7.82) | −8.78 | (8.56) |
| AHTs | 2 | n | 17 | | 7 | | 14 | | 14 | | 20 | | 14 | |
| | | BL | 84.53 | (5.44) | 86.00 | (5.78) | 81.61 | (5.93) | 84.32 | (12.72) | 83.43 | (6.54) | 82.61 | (7.68) |
| | | Wk 8 | 81.31 | (9.05) | 79.67 | (11.78) | 79.31 | (15.91) | 80.80 | (18.35) | 76.40 | (10.12) | 74.09 | (4.66) |
| | | CFB | −3.56 | (8.99) | −5.25 | (7.78) | −2.12 | (15.46) | −4.60 | (10.04) | −7.03 | (9.50) | −8.09 | (8.81) |
| | ≥3 | n | 13 | | 15 | | 16 | | 9 | | 8 | | 16 | |
| | | BL | 81.85 | (11.90) | 79.47 | (7.58) | 76.56 | (8.90) | 79.39 | (5.69) | 88.50 | (4.38) | 75.59 | (10.12) |
| | | Wk 8 | 81.23 | (10.24) | 73.15 | (8.93) | 72.07 | (9.26) | 82.25 | (16.96) | 77.88 | (9.58) | 72.14 | (12.08) |
| | | CFB | −0.62 | (7.57) | −6.12 | (9.49) | −5.03 | (7.45) | 1.75 | (16.61) | −10.63 | (8.12) | −3.39 | (6.60) |
| ACEi/ | Yes | n | 22 | | 17 | | 27 | | 18 | | 20 | | 23 | |
| ARBs | | BL | 83.57 | (9.59) | 82.91 | (7.06) | 78.91 | (8.00) | 83.39 | (11.47) | 85.55 | (6.70) | 77.74 | (8.97) |
| | | Wk 8 | 81.32 | (10.11) | 74.93 | (9.86) | 76.24 | (13.40) | 83.79 | (17.89) | 76.05 | (8.09) | 73.40 | (9.37) |
| | | CFB | −2.25 | (8.51) | −6.33 | (9.02) | −2.88 | (11.56) | −0.71 | (13.94) | −9.50 | (8.30) | −4.58 | (8.10) |

| | | | | | | | |
|---|---|---|---|---|---|---|---|
| No | n | 8 | 5 | 3 | 5 | 8 | 7 |

| | | | | | | | | | | | | |
|---|---|---|---|---|---|---|---|---|---|---|---|---|
| BL | 82.81 | (6.46) | 76.90 | (8.23) | 79.00 | (9.18) | 78.80 | (6.64) | 83.19 | (5.47) | 82.57 | (11.38) |
| Wk 8 | 81.14 | (7.54) | 76.25 | (12.37) | 68.67 | (8.33) | 73.25 | (13.50) | 78.75 | (13.72) | 71.40 | (10.62) |
| CFB | −2.21 | (8.58) | −4.00 | (8.79) | −10.33 | (13.25) | −5.50 | (11.85) | −4.44 | (10.67) | −9.00 | (6.21) |

Abbreviations: AA, African American; ARB, angiotensin receptor blocker; ACEi, angiotensin converting enzyme inhibitor; AHT, antihypertensive; BID, twice daily; BL, baseline; BMI, body mass index; CFB change from baseline; kg, kilogram; m, meters; QD, once daily; SBP, systolic blood pressure; SD, standard deviation; Wk 8, Week 8; yr, years.

TABLE 33
Mean (SD) Change from Baseline at Week 8 in Automated
Office-Measured Diastolic Blood Pressure, by
Subgroup, Part 2 (Full Analysis Set)

| Cate- | Sub- | | Placebo | | 100 mg QD | |
|---|---|---|---|---|---|---|
| gory | group | Visit | (N = 6) | | (N = 31) | |
| Sex | Male | n | 2 | | 10 | |
| | | BL | 80.75 | (7.42) | 79.50 | (8.18) |
| | | Wk 8 | 74.50 | (17.68) | 75.00 | (6.85) |
| | | CFB | −6.25 | (10.25) | −4.50 | (9.14) |
| | Female | n | 4 | | 21 | |
| | | BL | 77.00 | (8.44) | 77.36 | (7.48) |
| | | Wk 8 | 77.00 | (9.70) | 70.75 | (8.89) |
| | | CFB | 0.00 | (7.71) | −6.28 | (11.69) |
| Age | <65 | n | 4 | | 8 | |
| (yr) | | BL | 82.38 | (2.75) | 82.88 | (4.92) |
| | | Wk 8 | 82.25 | (7.09) | 72.50 | (10.73) |
| | | CFB | −0.13 | (7.69) | −10.38 | (12.61) |
| | 65-79 | n | 2 | | 22 | |
| | | BL | 70.00 | (7.78) | 76.57 | (7.90) |
| | | Wk 8 | 64.00 | (2.83) | 72.38 | (7.70) |
| | | CFB | −6.00 | (10.61) | −3.83 | (10.02) |
| | ≥80 | n | 0 | | 1 | |
| | | BL | | | 72.00 | (NE) |
| | | Wk 8 | | | 65.00 | (NE) |
| | | CFB | | | −7.00 | (NE) |
| Race | Black or | n | 2 | | 6 | |
| | AA | BL | 80.25 | (0.35) | 76.67 | (7.74) |
| | | Wk 8 | 85.00 | (2.83) | 72.00 | (10.79) |
| | | CFB | 4.75 | (3.18) | −4.67 | (11.45) |
| | Other | n | 4 | | 25 | |
| | | BL | 77.25 | (9.58) | 78.38 | (7.74) |
| | | Wk 8 | 71.75 | (10.97) | 72.21 | (7.97) |
| | | CFB | −5.50 | (7.86) | −5.94 | (10.85) |
| BMI | <25 | n | 1 | | 2 | |
| (kg/ | | BL | 83.00 | (NE) | 78.25 | (12.37) |
| m²) | | Wk 8 | 72.00 | (NE) | 77.00 | (14.14) |
| | | CFB | −11.00 | (NE) | −1.25 | (1.77) |
| | 25-30 | n | 0 | | 15 | |
| | | BL | | | 76.50 | (7.14) |
| | | Wk 8 | | | 70.40 | (10.22) |
| | | CFB | | | −6.10 | (12.04) |
| | >30 | n | 5 | | 14 | |
| | | BL | 77.30 | (8.07) | 79.68 | (7.90) |
| | | Wk 8 | 77.00 | (12.06) | 73.46 | (4.70) |
| | | CFB | −0.30 | (7.75) | −5.88 | (10.38) |
| Seated | Low | n | 4 | | 10 | |
| SBP | | BBL | 75.88 | (8.20) | 79.60 | (6.56) |
| Tertile | | Wk 8 | 70.75 | (9.14) | 72.33 | (10.76) |
| | | CFB | −5.13 | (8.30) | −6.78 | (13.35) |
| | Middle | n | 1 | | 10 | |
| | | BL | 86.00 | (NE) | 78.50 | (7.42) |
| | | Wk 8 | 87.00 | (NE) | 72.10 | (4.41) |
| | | CFB | 1.00 | (NE) | −6.40 | (9.59) |
| | High | n | 1 | | 11 | |
| | | BL | 80.00 | (NE) | 76.23 | (8.99) |
| | | Wk 8 | 87.00 | (NE) | 72.09 | (9.71) |
| | | CFB | 7.00 | (NE) | −4.14 | (10.37) |
| AHTs | 2 | n | 4 | | 20 | |
| | | BL | 75.88 | (8.20) | 78.10 | (7.16) |
| | | Wk 8 | 70.75 | (9.14) | 71.58 | (9.17) |
| | | CFB | −5.13 | (8.30) | −6.21 | (10.33) |
| | ≥3 | n | 2 | | 11 | |
| | | BL | 83.00 | (4.24) | 77.95 | (8.82) |
| | | Wk 8 | 87.00 | (0.00) | 73.18 | (7.17) |
| | | CFB | 4.00 | (4.24) | −4.77 | (11.98) |
| ACEi/ | Yes | n | 5 | | 30 | |
| ARBs | | BL | 77.80 | (8.39) | 77.75 | (7.59) |
| | | Wk 8 | 74.80 | (11.69) | 71.66 | (8.05) |
| | | CFB | −3.00 | (8.81) | −5.88 | (10.92) |
| | No | n | 1 | | 1 | |
| | | BL | 80.50 | (NE) | 87.00 | (NE) |
| | | Wk 8 | 83.00 | (NE) | 87.00 | (NE) |
| | | CFB | 2.50 | (NE) | 0.00 | (NE) |
| PRA | 1-5 ng/ | n | 5 | | 14 | |
| | mL/hr | BL | 78.80 | (8.34) | 74.07 | (5.74) |
| | | Wk 8 | 79.00 | (9.51) | 71.93 | (6.65) |
| | | CFB | 0.20 | (6.70) | −2.14 | (8.23) |
| | >5 ng/ | n | 1 | | 8 | |
| | mL/hr | BL | 75.50 | (NE) | 83.19 | (5.06) |
| | | Wk 8 | 62.00 | (NE) | 71.29 | (11.40) |
| | | CFB | −13.50 | (NE) | −11.79 | (12.93) |

Abbreviations: AA, African American; ARB, angiotensin receptor blocker; ACEi, angiotensin converting enzyme inhibitor; AHT, antihypertensive; BL, baseline; BMI, body mass index; CFB change from baseline; hr, hour; kg, kilogram; m, meters; mL, milliliter; ng, nanograms; PRA, plasma renin activity; QD, once daily; SBP, systolic blood pressure; SD, standard deviation; Wk 8, Week 8; yr, years.

A modest increase in the AHT effect of lorundrostat was apparent with concomitant use of thiazide diuretics in Part 1 (Table 34), but this was not present in Part 2 (Table 35). In Part 1, a statistically significant placebo-adjusted reduction in AOBP DBP was achieved at Week 8 in patients with thiazide diuretic use at baseline for the 50 mg QD cohort (−7.7 mmHg, p=0.0291), but not for any of the dose cohorts in patients not on thiazide diuretics at baseline.

TABLE 34
Mean (SD) Change from Baseline at Week 8 in Automated Office-Measured Diastolic
Blood Pressure, by Thiazide Diuretic Use at Baseline, Part 1 (Full Analysis Set)

| Sub- | | Placebo | | 12.5 mg BID | | 25 mg BID | | 12.5 mg QD | | 50 mg QD | | 100 mg QD | |
|---|---|---|---|---|---|---|---|---|---|---|---|---|---|
| group | Visit | (N = 30) | | (N = 22) | | (N = 30) | | (N = 23) | | (N = 28) | | (N = 30) | |
| Yes | n | 16 | | 13 | | 18 | | 12 | | 16 | | 17 | |
| | BL | 82.31 | (10.80) | 78.73 | (5.57) | 78.56 | (8.71) | 78.92 | (5.86) | 86.50 | (4.60) | 80.50 | (9.51) |
| | Week 8 | 80.69 | (9.82) | 72.67 | (9.15) | 76.12 | (15.60) | 74.78 | (8.58) | 75.94 | (11.89) | 73.40 | (8.98) |
| | CFB | −1.63 | (8.57) | −7.33 | (8.79) | −2.12 | (13.83) | −5.11 | (6.26) | −10.56 | (9.49) | −6.43 | (8.79) |
| No | n | 14 | | 9 | | 12 | | 11 | | 12 | | 13 | |
| | BL | 84.57 | (5.81) | 85.61 | (8.54) | 79.46 | (6.99) | 86.18 | (13.47) | 82.71 | (7.86) | 76.73 | (9.65) |
| | Week 8 | 82.00 | (9.25) | 79.57 | (10.75) | 74.36 | (8.31) | 88.11 | (21.40) | 78.00 | (6.42) | 72.40 | (10.54) |
| | CFB | −3.00 | (8.39) | −3.29 | (8.80) | −6.09 | (7.34) | 1.56 | (17.70) | −4.71 | (7.77) | −4.00 | (6.33) |

TABLE 35
Mean (SD) Change from Baseline at Week 8 in Automated
Office-Measured Diastolic Blood Pressure, by Thiazide
Diuretic Use at Baseline, Part 2 (Full Analysis Set)

| Sub- | | Placebo | 100 mg QD |
|---|---|---|---|
| group | Visit | (N = 6) | (N = 31) |
| Yes | n | 5 | 19 |

| | | | | |
|---|---|---|---|---|
| BL | 78.80 | (8.34) | 79.00 | (7.73) |
| Week 8 | 79.00 | (9.51) | 74.67 | (7.39) |
| CFB | 0.20 | (6.70) | −4.06 | (10.70) |

| | | | |
|---|---|---|---|
| No | n | 1 | 12 |

| | | | | |
|---|---|---|---|---|
| BL | 75.50 | (NE) | 76.54 | (7.58) |
| Week 8 | 62.00 | (NE) | 68.42 | (8.71) |
| CFB | −13.50 | (NE) | −8.13 | (10.89) |

### Pharmacokinetic Response

Systemic exposure to lorundrostat, expressed as Cₘₐₓ or AUC increased with increasing dose, although there was moderately high inter-subject variability. For the dose of 100 mg QD in Cohort 1 and Cohort 2, steady-state (Day 28) Cₘₐₓ was 1620 ng/mL and 1360 ng/mL, respectively (mean approximately 1490 ng/mL) and AUC₀₋₂₄ was 14900 ng*h/mL and 7480 ng*h/mL, respectively (mean approximately 11190 ng*h/mL). Other key parameters are summarized in Table 36. Values of trough plasma concentrations are shown in Table 37.

TABLE 36
Summary of Key Steady-State Pharmacokinetic Parameters Following Administration of Lorundrostat

| Parameter | Statistics | 12.5 mg BID | | 25 mg BID | | 12.5 mg QD | | 50 mg QD | | 100 mg QD | | 100 mg QD | |
|---|---|---|---|---|---|---|---|---|---|---|---|---|---|
| (Unit) | (Day 28) | (N = 22) | | (N = 30) | | (N = 23) | | (N = 28) | | Part 1 (N = 30) | | Part 2 (N = 31) | |
| AUC₀₋₂₄ | n (missing) | 3 | (19) | 7 | (23) | 5 | (18) | 3 | (25) | 4 | (26) | 11 | (20) |
| (hr*ng/mL) | Mean (SD) | 992 | (398) | 1760 | (774) | 1020 | (1090) | 3390 | (1260) | 14900 | (11200) | 7480 | (3070) |
| | % CV | 40.1 | | 43.9 | | 107 | | 37.3 | | 75.3 | | 41.1 | |
| | Geo Mean | 940 | | 1640 | | 726 | | 3230 | | 10400 | | 6920 | |
| | Geo CV (%) | 42.1 | | 41.8 | | 102 | | 40.3 | | 164 | | 44.5 | |
| | Median | 922 | | 1670 | | 498 | | 3320 | | 15000 | | 7090 | |
| | Min; Max | 634; 1420 | | 931; 3330 | | 397; 2940 | | 2160; 4680 | | 2140; 27600 | | 2870; 14700 | |
| Cₘₐₓ | n (missing) | 7 | (15) | 16 | (14) | 9 | (14) | 6 | (22) | 8 | (22) | 17 | (14) |
| (ng/mL) | Mean (SD) | 142 | (28.3) | 361 | (175) | 153 | (129) | 518 | (223) | 1620 | (867) | 1360 | (913) |
| | % CV | 19.8 | | 48.6 | | 84.6 | | 43.1 | | 53.5 | | 67.2 | |
| | Geo Mean | 140 | | 325 | | 120 | | 477 | | 1370 | | 1000 | |
| | Geo CV (%) | 21 | | 50.9 | | 78.2 | | 47.8 | | 78.9 | | 124 | |
| | Median | 137 | | 335 | | 94.9 | | 488 | | 1590 | | 1250 | |
| | Min; Max | 99.8; 176 | | 112; 862 | | 56.1; 459 | | 266; 790 | | 331; 2890 | | 66.7; 3260 | |
| Tₘₐₓ | n (missing) | 7 | (15) | 16 | (14) | 9 | (14) | 6 | (22) | 8 | (22) | 17 | (14) |

| | | | | | | | |
|---|---|---|---|---|---|---|---|
| (hr) | Median | 2 | 2 | 1.08 | 2.01 | 1.02 | 1 |
| | Min; Max | 1.00; 3.00 | 1.00; 4.02 | 1.00; 3.00 | 1.00; 4.05 | 1.00; 2.08 | 1.00; 4.00 |

Abbreviations: AUC, area under the curve; BID, twice daily; CV, coefficient of variation; geo, geometric; hr, hours; mg, milligrams; mL, milliliters; N, number of subjects; n, number of observations; ng, nanograms; QD, once daily.

TABLE 37
Summary of Trough Plasma Concentrations (ng/mL) Following Administration of Lorundrostat

| | | 12.5 mg BID | | 25 mg BID | | 12.5 mg QD | | 50 mg QD | | 100 mg QD | | 100 mg QD | |
|---|---|---|---|---|---|---|---|---|---|---|---|---|---|
| Visit | Statistic | (N = 22) | | (N = 30) | | (N = 23) | | (N = 28) | | Part 1 (N = 30) | | Part 2 (N = 31) | |
| Day 7 | n (missing) | 20 | (2) | 27 | (3) | 22 | (1) | 26 | (2) | 23 | (7) | 30 | (1) |
| | Mean (SD) | 52.3 | (83.3) | 94.2 | (137) | 11.6 | (18.2) | 93.1 | (216) | 299 | (571) | 151 | (346) |
| | % CV | 159 | | 146 | | 156 | | 233 | | 191 | | 228 | |
| | Geo Mean | 20.4 | | 49.6 | | 6.55 | | 16.6 | | 53.4 | | 35.1 | |
| | Geo CV (%) | 573 | | 364 | | 306 | | 2680 | | 2080 | | 625 | |
| | Median | 26.1 | | 52 | | 5.52 | | 12.1 | | 28.2 | | 29.8 | |
| | Min; Max | 0.00; 366 | | 0.00; 603 | | 0.00; 61.2 | | 0.00; 909 | | 0.00; 2060 | | 0.00; 1370 | |
| Day 28 | n (missing) | 16 | (6) | 23 | (7) | 17 | (6) | 22 | (6) | 22 | (8) | 23 | (8) |
| | Mean (SD) | 33.4 | (37.5) | 109 | (161) | 7.91 | (10.9) | 87.4 | (184) | 303 | (603) | 26.4 | (29.2) |
| | % CV | 112 | | 148 | | 138 | | 210 | | 199 | | 110 | |
| | Geo Mean | 10.7 | | 62.1 | | 5.77 | | 46.8 | | 50.7 | | 12.9 | |
| | Geo CV (%) | 1300 | | 310 | | 297 | | 219 | | 3510 | | 377 | |
| | Median | 27.3 | | 56.3 | | 4.1 | | 20.9 | | 38.6 | | 19.9 | |
| | Min; Max | 0.143; 150 | | 0.00; 787 | | 0.00; 37.9 | | 0.00; 675 | | 0.00; 1980 | | 0.00; 127 | |
| Day 56 | n (missing) | 13 | (9) | 21 | (9) | 12 | (11) | 25 | (3) | 19 | (11) | 26 | (5) |
| | Mean (SD) | 40.4 | (58.9) | 104 | (167) | 39.9 | (61.3) | 155 | (314) | 258 | (487) | 23.2 | (24.6) |

| | | | | | | |
|---|---|---|---|---|---|---|
| % CV | 146 | 160 | 154 | 203 | 189 | 106 |
| Geometric Mean | 18.5 | 46 | 22.1 | 38.1 | 72.1 | 20.3 |
| Geometric CV (%) | 732 | 447 | 540 | 698 | 1060 | 178 |
| Median | 21.6 | 32.3 | 7.6 | 18 | 30.4 | 20 |
| Min; Max | 0.00; 201 | 0.00; 720 | 0.00; 186 | 0.00; 1290 | 0.00; 1900 | 0.00; 110 |

Concentrations are in units of ng/mL.
Abbreviations: BID, twice daily; CV, coefficient of variation; geo, geometric; mg, milligrams; mL, milliliters; N, number of subjects; n, number of observations; ng, nanograms; QD, once daily.

### Pharmacodynamic Response

At baseline in Part 1, active arm mean serum cortisol levels ranged between 9.443 μg/dL and 11.272 μg/dL, compared with 10.507 μg/dL for placebo (Table 38). No consistent substantial changes in serum cortisol levels were observed over the course of treatment and follow-up relative to baseline values with percent change from baseline values of 3.8% to 19.2% (active) vs −0.8% (placebo) at Week 4, 20.6% to 51.4% (active) vs 37.7% (placebo) at Week 8, and −1.2% to 22.7% (active) vs 17.1% (placebo) at Week 12. A similar pattern was observed in Part 2, with active arm serum cortisol baseline levels of 11.118 μg/dL in the 100 mg QD dose group and no substantial changes observed over the course of treatment and subsequent follow-up. Percent change from baseline was 20.1% at Week 4, 153.4% at Week 8, and 1.5% at Week 12 (Table 39).

Transient increases in 11-deoxycorticosterone and 11-deoxycortisol were seen in Part 1 in the active treatment arms at Week 4 compared with placebo, albeit with large variation within each individual dose cohort. Values returned to a similar range as placebo at Week 12 (Table 38). In Part 2, 11-deoxycorticosterone and 11-deoxycortisol were similar to baseline in subjects treated with lorundrostat 100 mg QD at Week 4 and Week 10 (Table 39).

In Part 1, aldosterone levels generally decreased at Week 4 relative to baseline in lorundrostat-treated subjects (−40.1% to 7.4%) compared with those who received placebo (3.6%), and remained lower relative to placebo at Week 12 (−26.6% to 15.9% vs 38.1%, respectively; Table 38). In Part 2, aldosterone levels remained consistent although lower than baseline through Week 4 and Week 10 in subjects receiving lorundrostat. (Table 39).

Renin activity, expressed as a percent change from baseline, increased in all subjects treated with lorundrostat, irrespective of dose and baseline PRA values, reaching highest levels at Week 4 and remaining elevated compared with baseline at Week 12 (Part 1) and Week 10 (Part 2) (Table 38 and Table 39, respectively). Placebo values in Part 1 remained relatively consistent with baseline.

TABLE 38
Mean (SD) Observed and Mean (SD) Percent Change from Baseline
in Pharmacodynamic Parameters, Part 1 (PK/PD Analysis Set)

| | | Placebo | | 12.5 mg BID | | 25 mg BID | |
|---|---|---|---|---|---|---|---|
| Parameter | Visit | (N = 30) | | (N = 22) | | (N = 30) | |
| Cortisol | BL | 10.507 | (3.444) | 10.195 | (2.979) | 10.577 | (2.506) |
| (μg/dL) | Wk 4 | 10.248 | (3.673) | 10.895 | (3.859) | 12.311 | (5.180) |
| | % CFB | −0.81 | (31.53) | 10.26 | (45.03) | 19.20 | (49.39) |
| | Wk 8 | 14.190 | (8.589) | 12.316 | (7.649) | 15.525 | (8.258) |
| | % CFB | 37.72 | (89.31) | 20.56 | (73.01) | 49.57 | (77.17) |
| | Wk 12 | 11.800 | (4.620) | 9.978 | (2.742) | 12.559 | (4.287) |
| | % CFB | 17.13 | (39.18) | −1.22 | (21.10) | 22.65 | (41.94) |
| 11-Deoxy | BL | 8.1 | (0.7) | 8.0 | (0.0) | 8.0 | (0.0) |
| corticosterone | Wk 4 | 8.0 | (0.0) | 9.0 | (3.0) | 13.7 | (13.9) |
| (ng/dL) | % CFB | −1.15 | (6.18) | 12.50 | (37.50) | 71.63 | (173.69) |
| | Wk 12 | 8.0 | (0.0) | 8.0 | (0.0) | 8.0 | (0.0) |
| | % CFB | −1.15 | (6.18) | 0.00 | (0.00) | 0.00 | (0.00) |
| 11-Deoxy | BL | 26.24 | (16.08) | 28.36 | (17.85) | 25.87 | (11.45) |
| cortisol | Wk 4 | 28.41 | (28.72) | 24.89 | (18.99) | 35.23 | (33.00) |
| (ng/dL) | % CFB | −0.23 | (48.42) | 22.56 | (118.56) | 40.12 | (118.32) |
| | Wk 12 | 31.03 | (18.04) | 20.94 | (10.27) | 31.55 | (26.82) |
| | % CFB | 32.98 | (72.53) | −14.58 | (33.13) | 26.81 | (72.59) |
| Aldosterone | BL | 6.475 | (3.419) | 6.636 | (3.357) | 6.558 | (4.454) |
| (ng/dL) | Wk 4 | 6.768 | (4.779) | 4.447 | (3.681) | 3.192 | (3.274) |
| | % CFB | 3.58 | (52.63) | −37.86 | (35.66) | 1.66 | (233.60) |
| | Wk 12 | 7.914 | (5.943) | 6.765 | (4.085) | 4.983 | (3.432) |
| | % CFB | 38.09 | (84.56) | 1.97 | (66.85) | −10.10 | (66.83) |
| Renin | BL | 0.536 | (0.437) | 1.299 | (1.898) | 1.104 | (2.239) |
| Activity | Wk 4 | 0.687 | (0.973) | 5.989 | (11.272) | 2.860 | (4.219) |
| (μg/L/hr) | % CFB | 24.53 | (87.34) | 838.89 | (2411.4) | 344.07 | (480.75) |
| | Wk 12 | 0.615 | (1.033) | 5.118 | (11.751) | 2.898 | (8.323) |
| | % CFB | 3.53 | (86.37) | 212.37 | (402.15) | 198.91 | (423.17) |

| | 12.5 mg QD | | 50 mg QD | | 100 mg QD | |
|---|---|---|---|---|---|---|
| Parameter | (N = 23) | | (N = 28) | | (N = 30) | |
| Cortisol | 9.443 | (4.111) | 10.807 | (2.733) | 11.272 | (3.548) |
| (μg/dL) | 9.305 | (4.164) | 10.875 | (3.368) | 11.842 | (4.350) |
| | 7.47 | (37.82) | 3.83 | (32.48) | 3.91 | (32.33) |
| | 12.511 | (8.784) | 15.650 | (7.102) | 16.680 | (9.083) |
| | 41.84 | (90.37) | 51.35 | (78.86) | 47.29 | (92.65) |
| | 9.317 | (3.294) | 11.262 | (3.590) | 12.680 | (5.659) |
| | 18.06 | (48.59) | 6.91 | (35.36) | 8.40 | (30.09) |
| 11-Deoxy | 8.3 | (1.5) | 8.3 | (1.2) | 8.4 | (2.0) |
| corticosterone | 11.1 | (5.5) | 8.0 | (0.0) | 9.5 | (5.2) |
| (ng/dL) | 31.41 | (60.95) | −2.66 | (9.61) | 16.18 | (66.37) |
| | 9.2 | (4.9) | 8.5 | (2.4) | 8.4 | (2.0) |
| | 11.96 | (62.37) | 3.01 | (31.53) | 2.80 | (28.58) |
| 11-Deoxy | 29.07 | (26.29) | 31.14 | (20.96) | 29.97 | (20.53) |
| cortisol | 24.21 | (19.92) | 27.15 | (13.97) | 29.29 | (23.99) |
| (ng/dL) | 10.07 | (76.22) | 10.80 | (61.95) | 9.43 | (60.26) |
| | 31.56 | (29.77) | 26.15 | (18.89) | 31.17 | (25.98) |
| | 58.62 | (122.14) | 9.25 | (111.16) | 22.05 | (64.71) |
| Aldosterone | 7.065 | (3.728) | 6.205 | (3.777) | 6.533 | (3.298) |
| (ng/dL) | 6.132 | (4.606) | 3.923 | (5.283) | 4.000 | (4.252) |
| | 7.39 | (91.87) | −40.06 | (70.25) | −32.18 | (75.13) |
| | 6.833 | (4.743) | 5.385 | (4.087) | 4.563 | (2.868) |
| | 15.91 | (67.68) | −3.75 | (56.02) | −26.56 | (37.55) |
| Renin | 0.950 | (1.067) | 1.258 | (3.008) | 0.956 | (2.120) |
| Activity | 1.804 | (3.565) | 3.940 | (9.162) | 3.870 | (6.308) |
| (μg/L/hr) | 85.09 | (291.65) | 359.69 | (888.86) | 489.18 | (974.60) |
| | 2.429 | (5.048) | 3.374 | (7.008) | 1.653 | (2.830) |
| | 137.45 | (279.23) | 400.07 | (808.60) | 94.66 | (221.58) |

TABLE 39
Mean Observed (SD) and Mean (SD) Percent Change from Baseline
in Pharmacodynamic Parameters, Part 2 (PK/PD Analysis Set)

| | | Placebo | | 100 mg QD | |
|---|---|---|---|---|---|
| Parameter | Visit | (N = 6) | | (N = 31) | |
| Cortisol | BL | 10.508 | (2.655) | 11.118 | (3.465) |
| (ug/dL) | Wk 4 | 12.217 | (3.898) | 13.464 | (5.061) |
| | % CFB | 20.42 | (39.75) | 20.10 | (37.25) |
| | Wk 8 | 28.050 | (5.400) | 25.393 | (6.743) |
| | % CFB | 190.53 | (122.33) | 153.35 | (106.59) |
| | Wk 10 | 11.917 | (2.934) | 10.763 | (4.677) |
| | % CFB | 14.93 | (16.92) | 1.45 | (37.30) |
| 11-Deoxycortico- | BL | 8.00 | (0.00) | 10.71 | (8.25) |
| sterone | Wk 4 | 9.33 | (3.27) | 10.28 | (4.28) |
| (ng/dL) | % CFB | 16.67 | (40.82) | 6.02 | (40.58) |
| | Wk 10 | 8.00 | (0.00) | 8.00 | (0.00) |
| | % CFB | 0.00 | (0.00) | −8.78 | (23.71) |
| 11-Deoxycortisol | BL | 14.17 | (6.82) | 40.79 | (55.80) |
| (ng/dL) | Wk 4 | 21.83 | (14.92) | 35.96 | (29.50) |
| | % CFB | 65.30 | (110.59) | 22.95 | (81.16) |
| | Wk 10 | 16.17 | (10.96) | 27.60 | (25.66) |
| | % CFB | 22.20 | (62.92) | −2.13 | (71.17) |
| Aldosterone | BL | 5.917 | (1.800) | 6.435 | (3.397) |
| (ng/dL) | Wk 4 | 6.000 | (3.225) | 5.060 | (4.835) |
| | % CFB | 11.78 | (77.75) | −6.05 | (93.64) |
| | Wk 10 | 5.667 | (2.875) | 4.929 | (3.319) |
| | % CFB | 1.35 | (56.01) | −4.06 | (74.89) |
| Renin Activity | BL | 5.531 | (8.663) | 5.417 | (7.052) |
| (ug/L/hr) | Wk 4 | 4.097 | (2.492) | 12.642 | (12.078) |
| | % CFB | 44.08 | (77.01) | 811.54 | (2089.82) |
| | Wk 10 | 5.628 | (8.593) | 7.602 | (7.419) |
| | % CFB | 9.73 | (63.97) | 192.62 | (376.08) |

Subjects dosed with lorundrostat in Part 1, irrespective of dose, exhibited greater declines in eGFR relative to placebo as early as 1 week after the initiation of treatment that persisted at a consistent level until Week 8 (Table 40). The greatest reductions relative to baseline were seen in the 100 mg QD dose group, reaching a mean maximum decline of 10.91 mL/min/1.73 m² at Week 2 (range: −6.80 mL/min/1.73 m² to −10.91 mL/min/1.73 m² over 8 weeks of treatment). In contrast, mean eGFR for the Placebo group was relatively stable throughout treatment, reaching a mean maximal reduction of −2.80 mL/min/1.73 m² at Week 2 (range: −0.83 mL/min/1.73 m² to −2.80 mL/min/1.73 m²). Estimated glomerular filtration rate was estimated using the CKD-EPI equation.

Similar trends were seen with mean eGFR for the 100 mg QD treatment group in Part 2, with a rapid initial decline following a lesser reduction persisting out to Week 8 (Table 41).

TABLE 40
Mean (SD) Observed and Mean (SD) Change from Baseline in Estimated Glomerular
Filtration Rate (mL/min/1.73 m²), Part 1 (Safety Analysis Set)

| | Placebo | | 12.5 mg BID | | 25 mg BID | | 12.5 mg QD | | 50 mg QD | | 100 mg QD | |
|---|---|---|---|---|---|---|---|---|---|---|---|---|
| Visit | (N = 30) | | (N = 22) | | (N = 30) | | (N = 23) | | (N = 28) | | (N = 30) | |
| BL | 81.63 | (17.31) | 81.70 | (16.28) | 80.85 | (12.37) | 77.87 | (18.69) | 77.18 | (14.05) | 77.35 | (13.95) |
| Week 1 | 80.80 | (16.35) | 79.45 | (15.19) | 74.00 | (13.13) | 73.57 | (20.33) | 74.21 | (16.88) | 70.81 | (17.66) |
| CFB | −0.83 | (7.62) | −2.25 | (8.42) | −6.85 | (8.88) | −4.30 | (7.14) | −2.96 | (8.52) | −6.80 | (10.03) |
| Week 2 | 79.61 | (16.07) | 78.86 | (18.56) | 75.43 | (15.32) | 73.00 | (20.81) | 72.86 | (16.55) | 65.56 | (17.66) |
| CFB | −2.80 | (10.11) | −3.69 | (6.46) | −5.42 | (10.01) | −4.87 | (5.03) | −4.32 | (9.06) | −10.91 | (8.57) |
| Week 3 | 79.79 | (17.85) | 77.38 | (16.68) | 75.07 | (11.95) | 77.21 | (20.38) | 74.81 | (15.62) | 67.22 | (19.24) |
| CFB | −1.34 | (6.34) | −5.17 | (8.54) | −5.90 | (8.63) | −2.05 | (6.43) | −2.35 | (8.51) | −9.24 | (10.99) |
| Week 4 | 79.43 | (18.62) | 76.30 | (18.28) | 73.48 | (15.26) | 73.40 | (19.29) | 73.63 | (16.75) | 67.23 | (19.70) |
| CFB | −2.79 | (5.94) | −6.58 | (9.17) | −6.69 | (8.97) | −5.45 | (6.37) | −3.61 | (9.24) | −9.46 | (12.53) |
| Week 5 | 80.55 | (17.44) | 73.84 | (20.78) | 73.25 | (14.75) | 75.25 | (19.58) | 70.58 | (13.23) | 66.19 | (19.58) |
| CFB | −2.33 | (7.69) | −7.58 | (8.00) | −7.41 | (9.65) | −3.60 | (5.70) | −4.10 | (9.61) | −10.50 | (10.94) |
| Week 6 | 81.70 | (16.92) | 74.70 | (16.86) | 72.64 | (15.33) | 73.39 | (20.48) | 74.63 | (16.59) | 66.19 | (21.44) |
| CFB | −1.72 | (6.75) | −6.45 | (8.05) | −8.02 | (9.17) | −6.28 | (6.77) | −2.83 | (8.61) | −9.94 | (12.91) |
| Week 7 | 81.75 | (17.43) | 75.85 | (21.29) | 72.22 | (13.89) | 72.50 | (22.45) | 69.68 | (12.23) | 66.96 | (21.02) |
| CFB | −1.77 | (7.67) | −7.03 | (8.98) | −7.85 | (9.47) | −7.17 | (8.80) | −5.46 | (10.42) | −9.50 | (12.17) |
| Week 8 | 83.83 | (17.32) | 76.32 | (19.63) | 75.11 | (14.81) | 76.00 | (21.00) | 72.54 | (16.89) | 68.42 | (20.14) |
| CFB | 0.95 | (7.06) | −6.66 | (7.92) | −5.55 | (9.22) | −3.67 | (8.10) | −4.64 | (9.87) | −7.83 | (11.62) |

TABLE 41
Mean (SD) Observed and Mean (SD) Change from Baseline
in Estimated Glomerular Filtration Rate (mL/min/1.73
m²), Part 2 (Safety Analysis Set)

| | | |
|---|---|---|
| Visit | Placebo (N = 6) | 100 mg QD (N = 31) |

| | | | | |
|---|---|---|---|---|
| BL | 83.92 | (18.59) | 79.90 | (13.10) |
| Week 1 | 78.67 | (22.40) | 70.73 | (20.11) |
| CFB | −5.25 | (4.78) | −9.65 | (10.96) |
| Week 2 | 81.17 | (17.78) | 72.07 | (17.16) |
| CFB | −2.75 | (5.24) | −8.05 | (6.72) |
| Week 3 | 78.17 | (14.74) | 72.69 | (16.83) |
| CFB | −5.75 | (7.83) | −7.33 | (8.39) |
| Week 4 | 82.83 | (11.30) | 72.92 | (15.44) |
| CFB | −1.08 | (8.55) | −7.20 | (7.02) |
| Week 5 | 84.00 | (17.92) | 71.23 | (18.13) |
| CFB | 0.08 | (8.23) | −8.69 | (10.16) |
| Week 6 | 74.80 | (12.68) | 68.04 | (15.17) |
| CFB | −2.90 | (2.68) | −10.87 | (8.39) |
| Week 7 | 80.17 | (17.14) | 72.82 | (13.35) |
| CFB | −3.75 | (3.39) | −7.34 | (9.07) |
| Week 8 | 79.83 | (15.78) | 72.43 | (16.32) |
| CFB | −4.08 | (3.94) | −7.95 | (9.13) |

### Safety

At least one on-treatment TEAE was reported by 83 subjects in Part 1 (73 [54.9%] treated with lorundrostat and 10 [33.30%] treated with placebo) and by 20 subjects in Part 2 (19 [61.3%] treated with lorundrostat and 1 [16.7%] treated with placebo). Of these subjects, 30 (27 [20.30%] treated with active drug and 3 [10%] treated with placebo) from Part 1 and 9 (29.0% with lorundrostat only) from Part 2 experienced TEAEs that were considered at least possibly related to study drug.

Two (1.5%) subjects in Part 1 and one (3.2%) subject in Part 2 reported an SAE, one of which (a worsening of pre-existing hyponatremia) was considered possibly related to study drug. All events occurred in lorundrostat treated subjects.

Thirty-six subjects in Part 1 (35 [26.3%] treated with lorundrostat and 1 (3.3) treated with placebo) and four (12.9%) subjects treated with active drug in Part 2 reported at least one AESI.

Ten subjects in Part 1 and two subjects in Part 2, all in active treatment cohorts, experienced a TEAE that resulted in permanent discontinuation of study drug and 24 in Part 1 and 7 in Part 2 experienced a TEAE that resulted in dose modification.

No deaths were reported in either Part 1 or Part 2.

The most frequently reported events by PT were hyperkalemia (1 [3.3%] subject in the Part 1 Placebo cohort, 31 [23.3%] subjects in the Part 1 active treatment cohorts, and 8 [25.8%] subjects in the Part 2 100 mg QD cohort), eGFR decreased (1 [3.3%] subject in the Part 1 Placebo cohort, and 9 [6.8%] subjects in the Part 1 active treatment cohorts), and urinary tract infection (7 [5.3%] subjects in Part 1 active treatment cohorts, and 2 [6.5%] subjects in Part 2 100 mg QD cohort). Muscle spasms were reported by 3 (9.7%) subjects in the Part 2 100 mg QD cohort.

There was no apparent association between dose of study drug and frequency of on-treatment TEAEs, on-treatment TEAEs of at least moderate severity, or on-treatment TEAEs considered at least possible related to study drug by the Investigator.

Serum potassium levels and serum creatinine levels increased in the active treatment cohorts compared with placebo in both Parts 1 and 2 while serum sodium levels decreased. These changes are consistent with the mechanism of action of lorundrostat.

Seven subjects experienced transient elevated serum potassium (greater than 6.0 mmol/L), none of which were considered an SAE, and all rapidly resolved after intervention (study drug discontinuation or dose adjustment).

There were no notable findings in terms of vital signs, physical examination, or ECG parameters.

### Treatment-Emergent Adverse Events (TEAEs)

Overall, 28 subjects in Part 1 experienced on-treatment TEAEs of moderate intensity (3 [10.0%] subjects in the placebo cohort and 25 [18.8%] subjects in the active treatment cohorts) and one subject (0.8%) in the 12.5 mg QD cohort experienced a severe on-treatment TEAE (Table 42). The only on-treatment TEAEs of moderate severity reported in ≥2 subjects in any dose group by PT were hyperkalemia (4 [3.0%] subjects) and hypertension (3 [2.3%] subjects), all of which were reported in subjects in the active treatment cohorts. The one severe on-treatment TEAE was a case of hyperkalemia.

In Part 2, there were 12 (38.7%) subjects who experienced ≥1 moderate on-treatment TEAE, and one (3.2%) subject who experienced ≥1 severe on-treatment TEAE, all of whom were in the 100 mg QD cohort (Table 43). The only on-treatment TEAEs of moderate severity reported in ≥2 subjects were hyperkalemia (5 [16.1%] subjects) and muscle spasms (2 [6.5%] subjects. The one severe on-treatment TEAE was a case of hyponatremia.

TABLE 42
Severity of On-Treatment Treatment-Emergent Adverse Events by System Organ Class and Preferred
Term Reported by ≥2 Subjects in Any Treatment Group, Part 1 (Safety Analysis Set)

| | | | | | | | |
|---|---|---|---|---|---|---|---|
| System Organ | | | | | | | |
| Class | Placebo | Active | 12.5 mg BID | 25 mg BID | 12.5 mg QD | 50 mg QD | 100 mg QD |
| Preferred Term | (N = 30) | (N = 133) | (N = 22) | (N = 30) | (N = 23) | (N = 28) | (N = 30) |
| Severity | n (%) | n (%) | n (%) | n (%) | n (%) | n (%) | n (%) |

Subjects with any
On-treatment TEAEs

| Mild | 7 | (23.3) | 47 | (35.3) | 8 | (36.4) | 14 | (46.7) | 12 | (52.2) | 4 | (14.3) | 9 | (30.0) |
|---|---|---|---|---|---|---|---|---|---|---|---|---|---|---|
| Moderate | 3 | (10.0) | 25 | (18.8) | 5 | (22.7) | 4 | (13.3) | 3 | (13.0) | 6 | (21.4) | 7 | (23.3) |
| Severe | 0 | | 1 | (0.8) | 0 | | 0 | | 1 | (4.3) | 0 | | 0 | |
| Blood and | 0 | | 2 | (1.5) | 0 | | 2 | (6.7) | 0 | | 0 | | 0 | |
| lymphatic system | | | | | | | | | | | | | | |
| disorders | | | | | | | | | | | | | | |
| Mild | 0 | | 2 | (1.5) | 0 | | 2 | (6.7) | 0 | | 0 | | 0 | |
| Cardiac disorders | 0 | | 4 | (3.0) | 1 | (4.5) | 0 | | 2 | (8.7) | 1 | (3.6) | 0 | |
| Gastrointestinal | 2 | (6.7) | 6 | (4.5) | 0 | | 1 | (3.3) | 2 | (8.7) | 1 | (3.6) | 2 | (6.7) |
| disorders | | | | | | | | | | | | | | |
| Mild | 1 | (3.3) | 5 | (3.8) | 0 | | 1 | (3.3) | 1 | (4.3) | 1 | (3.6) | 2 | (6.7) |
| General disorders | 2 | (6.7) | 5 | (3.8) | 2 | (9.1) | 0 | | 1 | (4.3) | 2 | (7.1) | 0 | |
| and administration | | | | | | | | | | | | | | |
| site conditions | | | | | | | | | | | | | | |
| Mild | 2 | (6.7) | 2 | (1.5) | 1 | (4.5) | 0 | | 0 | | 1 | (3.6) | 0 | |
| Infections and | 1 | (3.3) | 11 | (8.3) | 1 | (4.5) | 3 | (10.0) | 3 | (13.0) | 2 | (7.1) | 2 | (6.7) |
| infestations | | | | | | | | | | | | | | |
| Mild | 0 | | 5 | (3.8) | 0 | | 2 | (6.7) | 3 | (13.0) | 0 | | 0 | |
| Moderate | 1 | (3.3) | 6 | (4.5) | 1 | (4.5) | 1 | (3.3) | 0 | | 2 | (7.1) | 2 | (6.7) |
| Urinary tract | 0 | | 7 | (5.3) | 0 | | 3 | (10.0) | 1 | (4.3) | 2 | (7.1) | 1 | (3.3) |
| infection | | | | | | | | | | | | | | |
| Mild | 0 | | 4 | (3.0) | 0 | | 2 | (6.7) | 1 | (4.3) | 1 | (3.6) | 0 | |
| Investigations | 4 | (13.3) | 21 | (15.8) | 6 | (27.3) | 3 | (10.0) | 3 | (13.0) | 1 | (3.6) | 8 | (26.7) |
| Mild | 4 | (13.3) | 17 | (12.8) | 5 | (22.7) | 2 | (6.7) | 3 | (13.0) | 1 | (3.6) | 6 | (20.0) |
| Moderate | 0 | | 4 | (3.0) | 1 | (4.5) | 1 | (3.3) | 0 | | 0 | | 2 | (6.7) |
| Blood creatinine | 0 | | 3 | (2.3) | 0 | | 0 | | 0 | | 0 | | 3 | (10.0) |
| increased | | | | | | | | | | | | | | |
| Mild | 0 | | 3 | (2.3) | 0 | | 0 | | 0 | | 0 | | 3 | (10.0) |
| Cortisol decreased | 1 | (3.3) | 3 | (2.3) | 2 | (9.1) | 1 | (3.3) | 0 | | 0 | | 0 | |
| Mild | 1 | (3.3) | 3 | (2.3) | 2 | (9.1) | 1 | (3.3) | 0 | | 0 | | 0 | |
| Glomerular | 1 | (3.3) | 9 | (6.8) | 3 | (13.6) | 0 | | 1 | (4.3) | 0 | | 5 | (16.7) |
| filtration rate | | | | | | | | | | | | | | |
| decreased | | | | | | | | | | | | | | |
| Mild | 1 | (3.3) | 8 | (6.0) | 3 | (13.6) | 0 | | 1 | (4.3) | 0 | | 4 | (13.3) |
| Metabolism and | 2 | (6.7) | 34 | (25.6) | 5 | (22.7) | 10 | (33.3) | 6 | (26.1) | 5 | (17.9) | 8 | (26.7) |
| nutrition disorders | | | | | | | | | | | | | | |
| Mild | 2 | (6.7) | 29 | (21.8) | 4 | (18.2) | 8 | (26.7) | 4 | (17.4) | 5 | (17.9) | 8 | (26.7) |
| Moderate | 0 | | 4 | (3.0) | 1 | (4.5) | 2 | (6.7) | 1 | (4.3) | 0 | | 0 | |
| Hyperkalaemia | 1 | (3.3) | 31 | (23.3) | 5 | (22.7) | 9 | (30.0) | 6 | (26.1) | 3 | (10.7) | 8 | (26.7) |
| Mild | 1 | (3.3) | 26 | (19.5) | 4 | (18.2) | 7 | (23.3) | 4 | (17.4) | 3 | (10.7) | 8 | (26.7) |
| Moderate | 0 | | 4 | (3.0) | 1 | (4.5) | 2 | (6.7) | 1 | (4.3) | 0 | | 0 | |
| Severe | 0 | | 1 | (0.8) | 0 | | 0 | | 1 | (4.3) | 0 | | 0 | |
| Nervous system | 0 | | 8 | (6.0) | 1 | (4.5) | 3 | (10.0) | 2 | (8.7) | 2 | (7.1) | 0 | |
| disorders | | | | | | | | | | | | | | |
| Mild | 0 | | 6 | (4.5) | 1 | (4.5) | 2 | (6.7) | 2 | (8.7) | 1 | (3.6) | 0 | |
| Dizziness | 0 | | 3 | (2.3) | 0 | | 0 | | 1 | (4.3) | 2 | (7.1) | 0 | |
| Headache | 0 | | 3 | (2.3) | 0 | | 2 | (6.7) | 0 | | 1 | (3.6) | 0 | |
| Mild | 0 | | 3 | (2.3) | 0 | | 2 | (6.7) | 0 | | 1 | (3.6) | 0 | |
| Psychiatric | 0 | | 3 | (2.3) | 1 | (4.5) | 0 | | 0 | | 0 | | 2 | (6.7) |
| disorders | | | | | | | | | | | | | | |
| Moderate | 0 | | 3 | (2.3) | 1 | (4.5) | 0 | | 0 | | 0 | | 2 | (6.7) |
| Vascular disorders | 1 | (3.3) | 9 | (6.8) | 2 | (9.1) | 1 | (3.3) | 3 | (13.0) | 2 | (7.1) | 1 | (3.3) |

| | | | | | | | | | | | | |
|---|---|---|---|---|---|---|---|---|---|---|---|---|
| Mild | 1 | (3.3) | 5 | (3.8) | 2 | (9.1) | 1 | (3.3) | 2 | (8.7) | 0 | 0 |

| | | | | | | | | | | | |
|---|---|---|---|---|---|---|---|---|---|---|---|
| Moderate | 0 | 4 | (3.0) | 0 | 0 | 1 | (4.3) | 2 | (7.1) | 1 | (3.3) |

| | | | | | | | | | | | |
|---|---|---|---|---|---|---|---|---|---|---|---|
| Hypertension | 0 | 5 | (3.8) | 0 | 1 | (3.3) | 2 | (8.7) | 2 | (7.1) | 0 |

| | | | | | | | | | | |
|---|---|---|---|---|---|---|---|---|---|---|
| Moderate | 0 | 3 | (2.3) | 0 | 0 | 1 | (4.3) | 2 | (7.1) | 0 |

| Orthostatic | 0 | 3 | (2.3) | 1 | (4.5) | 0 | 2 | (8.7) | 0 | 0 |
|---|---|---|---|---|---|---|---|---|---|---|
| hypotension | | | | | | | | | | |
| Mild | 0 | 3 | (2.3) | 1 | (4.5) | 0 | 2 | (8.7) | 0 | 0 |

Adverse events occurring in at least two subjects in any dose group by SOC and/or PT and/or severity are presented. SOCs with no PT and/or severity with at least two subjects are presented by SOC only. PTs with no severity with at least two subjects are presented by SOC and PT only.

TABLE 43
Severity of On-Treatment Treatment-Emergent Adverse Events
by System Organ Class and Preferred Term Reported by ≥2
Subjects in Any Treatment Group, Part 1 (Safety Analysis Set)

| System Organ Class | Placebo | 100 mg QD | |
|---|---|---|---|
| Preferred Term | (N = 6) | (N = 31) | |
| Severity | n (%) | n (%) | |
| Subjects with any On- | | | |
| treatment TEAEs | | | |
| Mild | 1 (16.7) | 6 | (19.4) |
| Moderate | 0 | 12 | (38.7) |
| Severe | 0 | 1 | (3.2) |
| Gastrointestinal disorders | 0 | 5 | (16.1) |
| Mild | 0 | 3 | (9.7) |
| Moderate | 0 | 2 | (6.5) |
| Diarrhoea | 0 | 2 | (6.5) |
| Mild | 0 | 2 | (6.5) |
| Nausea | 0 | 2 | (6.5) |
| Mild | 0 | 2 | (6.5) |
| Infections and infestations | 0 | 5 | (16.1) |
| Mild | 0 | 2 | (6.5) |
| Moderate | 0 | 3 | (9.7) |
| COVID-19 | 0 | 2 | (6.5) |
| Urinary tract infection | 0 | 2 | (6.5) |
| Investigations | 1 (16.7) | 2 | (6.5) |
| Metabolism and nutrition | 1 (16.7) | 10 | (32.3) |
| disorders | | | |
| Mild | 1 (16.7) | 2 | (6.5) |
| Moderate | 0 | 7 | (22.6) |
| Hyperkalaemia | 0 | 8 | (25.8) |
| Mild | 0 | 3 | (9.7) |
| Moderate | 0 | 5 | (16.1) |
| Hyponatraemia | 0 | 2 | (6.5) |
| Musculoskeletal and | 0 | 5 | (16.1) |
| connective tissue | | | |
| disorders | | | |
| Mild | 0 | 2 | (6.5) |
| Moderate | 0 | 3 | (9.7) |
| Muscle spasms | 0 | 3 | (9.7) |
| Moderate | 0 | 2 | (6.5) |
| Nervous system disorders | 0 | 2 | (6.5) |
| Mild | 0 | 2 | (6.5) |
| Dizziness | 0 | 2 | (6.5) |
| Mild | 0 | 2 | (6.5) |
| Renal and urinary | 0 | 2 | (6.5) |
| disorders | | | |
| Mild | 0 | 2 | (6.5) |
| Chronic kidney disease | 0 | 2 | (6.5) |
| Mild | 0 | 2 | (6.5) |

Adverse events occurring in at least two subjects in any dose group by SOC and/or PT and/or severity are presented. SOCs with no PT and/or severity with at least two subjects are presented by SOC only. PTs with no severity with at least two subjects are presented by SOC and PT only.

Ten (7.5%) subjects in Part 1, all in active treatment cohorts, experienced on-treatment TEAEs that resulted in permanent discontinuation of study drug (Table 44); hyperkalemia (5 [3.8%] subjects) was the only such event that was reported in ≥2 subjects.

In Part 2, there were 2 (6.5%) subjects who experienced an on-treatment TEAEs that resulted in permanent discontinuation of study drug, both of whom were in the 100 mg QD cohort ( ). No such events occurred in both subjects.

TABLE 44
On-Treatment Treatment-Emergent Adverse Events Leading to Permanent Discontinuation from
Study Drug by System Organ Class and Preferred Term, Part 1 (Safety Analysis Set)

| System Organ | Placebo | Active | | 12.5 mg BID | 25 mg BID | 12.5 mg QD | | 50 mg QD | 100 mg QD | |
|---|---|---|---|---|---|---|---|---|---|---|
| Class | (N = 30) | (N = 133) | | (N = 22) | (N = 30) | (N = 23) | | (N = 28) | (N = 30) | |
| Preferred Term | n (%) | n (%) | | n (%) | n (%) | n (%) | | n (%) | n (%) | |
| Subjects with any On- | 0 | 10 | (7.5) | 1 (4.5) | 1 (3.3) | 4 | (17.4) | 0 | 4 | (13.3) |
| treatment TEAEs leading to | | | | | | | | | | |
| withdrawal from study drug | | | | | | | | | | |
| Cardiac disorders | 0 | 1 | (0.8) | 0 | 0 | 1 | (4.3) | 0 | 0 | |
| Coronary artery disease | 0 | 1 | (0.8) | 0 | 0 | 1 | (4.3) | 0 | 0 | |
| Investigations | 0 | 1 | (0.8) | 0 | 0 | 0 | | 0 | 1 | (3.3) |
| Activated partial | 0 | 1 | (0.8) | 0 | 0 | 0 | | 0 | 1 | (3.3) |
| thromboplastin time | | | | | | | | | | |
| prolonged | | | | | | | | | | |
| Metabolism and nutrition | 0 | 5 | (3.8) | 0 | 1 (3.3) | 2 | (8.7) | 0 | 2 | (6.7) |
| disorders | | | | | | | | | | |
| Hyperkalaemia | 0 | 5 | (3.8) | 0 | 1 (3.3) | 2 | (8.7) | 0 | 2 | (6.7) |

| | | | | | | | | | |
|---|---|---|---|---|---|---|---|---|---|
| Nervous system disorders | 0 | 1 | (0.8) | 0 | 0 | 1 | (4.3) | 0 | 0 |
| Dizziness | 0 | 1 | (0.8) | 0 | 0 | 1 | (4.3) | 0 | 0 |

| Vascular disorders | 0 | 3 | (2.3) | 1 (4.5) | 1 (3.3) | 0 | 0 | 1 | (3.3) |
|---|---|---|---|---|---|---|---|---|---|
| Hypertension | 0 | 1 | (0.8) | 0 | 1 (3.3) | 0 | 0 | 0 | |
| Hypotension | 0 | 1 | (0.8) | 0 | 0 | 0 | 0 | 1 | (3.3) |

| | | | | | | | | |
|---|---|---|---|---|---|---|---|---|
| Orthostatic hypotension | 0 | 1 | (0.8) | 1 (4.5) | 0 | 0 | 0 | 0 |

TABLE 45
On-Treatment Treatment-Emergent Adverse Events Leading
to Permanent Withdrawal from Study Drug by System Organ
Class and Preferred Term, Part 2 (Safety Analysis Set)

| | Placebo | 100 mg QD |
|---|---|---|
| System Organ Class | (N = 6) | (N = 31) |
| Preferred Term | n (%) | n (%) |
| Subjects with any On- | 0 | 2 (6.5) |
| treatment TEAEs leading | | |
| to withdrawal from study | | |
| drug | | |
| Investigations | 0 | 1 (3.2) |
| Glomerular filtration rate | 0 | 1 (3.2) |
| decreased | | |
| Metabolism and nutrition | 0 | 1 (3.2) |
| disorders | | |
| Hyponatraemia | 0 | 1 (3.2) |
| Musculoskeletal and | 0 | 1 (3.2) |
| connective tissue | | |
| disorders | | |
| Muscle spasms | 0 | 1 (3.2) |

Change in Estimated Glomerular Filtration Rate (eGFR)

A dose-dependent and reversible reduction in eGFR was observed. This phenomenon has been reported with ACE/ARB and more recently SGLT2 inhibition due to reduced intra-glomerular pressure and felt to attenuate progression of hypertensive nephropathy. A graph showing the change in estimated glomerular filtration rate (eGFR) in different dosing cohorts is provided in FIG. 8.

### Clinical Laboratory Evaluations

There were no meaningful changes from baseline to Week 8 in bicarbonate, calcium, glucose, magnesium, phosphate, alanine aminotransferase (ALT), aspartate aminotransferase (AST), lactate dehydrogenase (LDH), alanine phosphatase (ALP), total or indirect bilirubin, albumin, protein, urate or urea nitrogen in Part 1 or Part 2. There were no meaningful changes from baseline to Week 8 in hematology and coagulation parameters in Part 1 or Part 2, or in urinalysis and spot/24-hour urine parameters.

### Serum Potassium

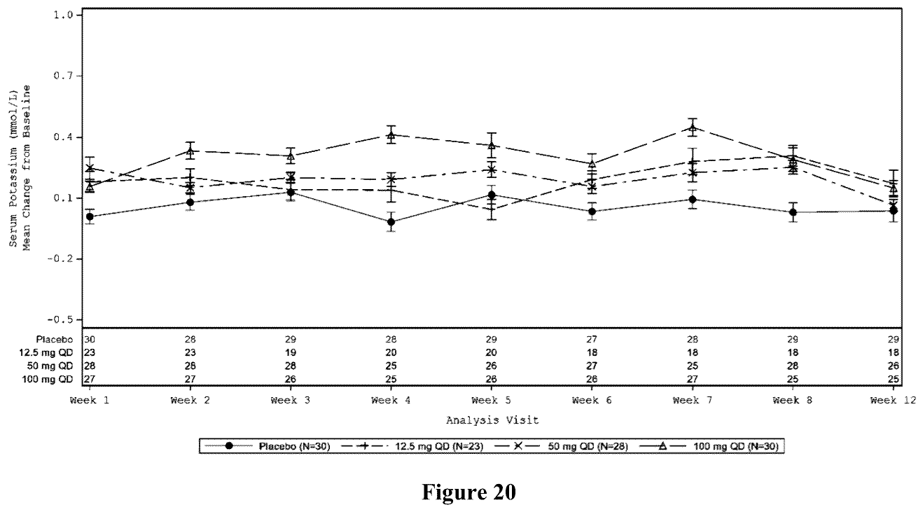

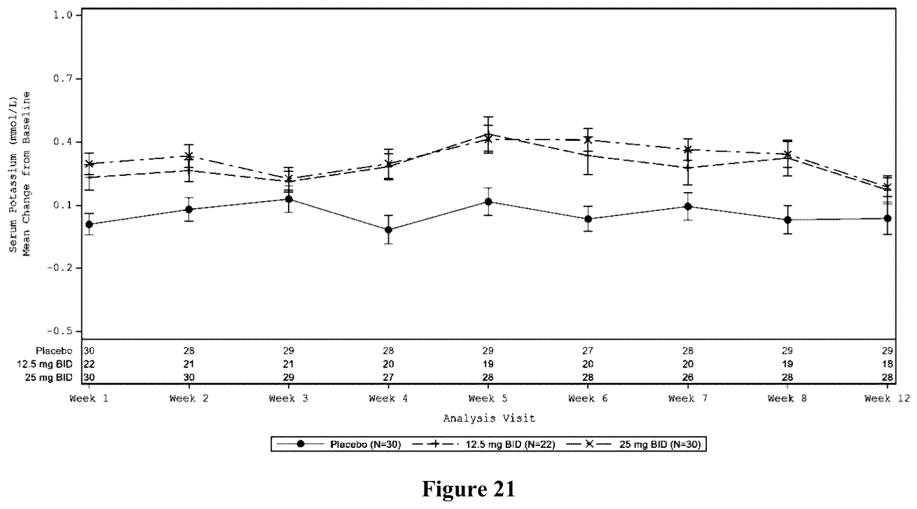

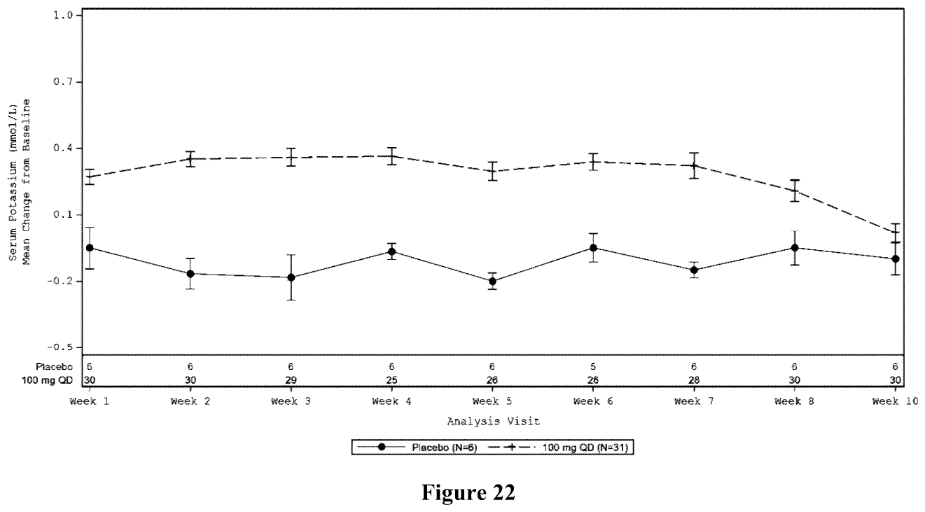

Serum potassium levels increased in the active treatment cohorts compared with the placebo cohort in both Parts 1 and 2, with the greatest increases seen in the 100 mg QD cohorts in Parts 1 and 2, and the two BID cohorts in Part 1 (FIG. 20, FIG. 21, and FIG. 22).

Change in group mean serum potassium (K+) is summarized in the table below.

TABLE 46

| | Part 1 | Part 2 | Pooled |
|---|---|---|---|
| Mean (mMol/L) | +0.50 | +0.23 | +0.35 |
| SD | 0.96 | 0.51 | 0.75 |
| N | 26* | 31 | 57 |
| SEM | 0.19 | 0.09 | 0.10 |

Number of individuals with verified or multiple increased serum potassium above normal range during treatment is shown in the table below.

TABLE 47

| | Mean Change | Mild | | Moderate | Severe |
|---|---|---|---|---|---|
| | from Baseline | 5.6-6.0 | | 6.1-6.5 | >6.5 |
| Serum K⁺ | to Wk 8 | mMol/L | | mMol/L | mMol/L |
| 50 mg QD | +0.25 mmol/L | 1 | (10.7%) | 0 | 1* (3.6%) |
| (n = 28) | | | | | |
| 100 mg QD | +0.29 mmol/L | 8 | (13.1%) | 1 (1.6%) | 1 (1.6%) |
| (n = 61) | | | | | |
| All Active | | 16 | (9.8%) | 4 (2.5%) | 2* |
| (n = 163) | | | | | |
| Placebo (n = 36) | | 1 | (2.8%) | 0 | 0 |

6/163 (3.6%) of subjects with one or more observed episodes of serum K⁺ >6.0 mmol/L. 5 of 6 judged not related to study medication.
*Measure in 1 subject was an isolated incident not verified by repeat measurement with study drug discontinuation (protocol deviation).

### Serum Sodium

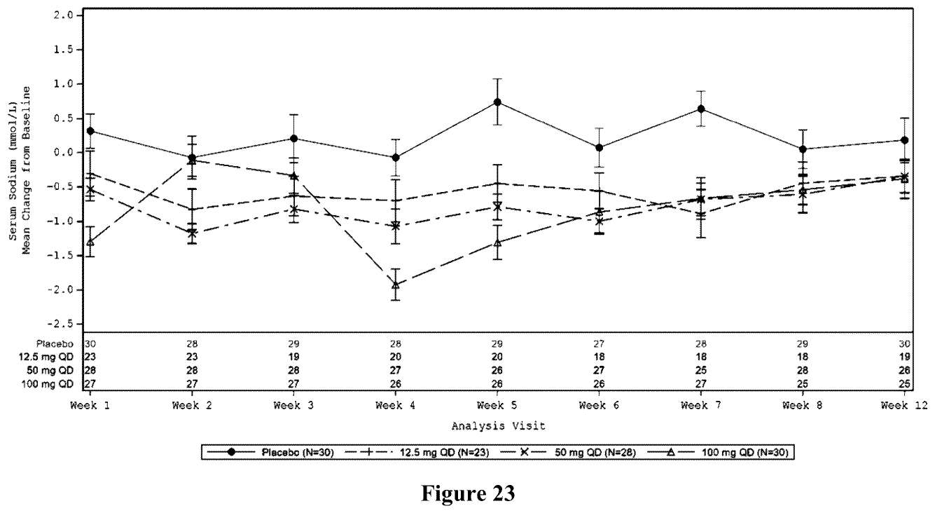

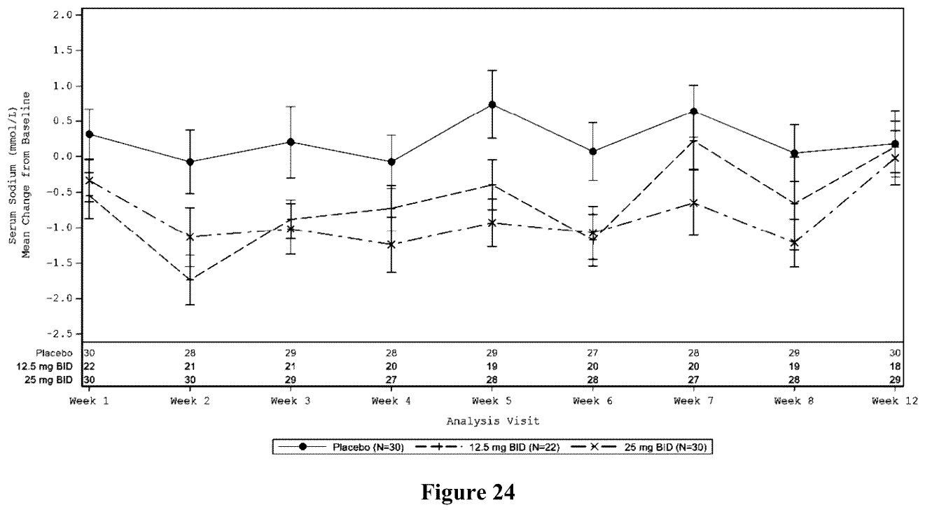

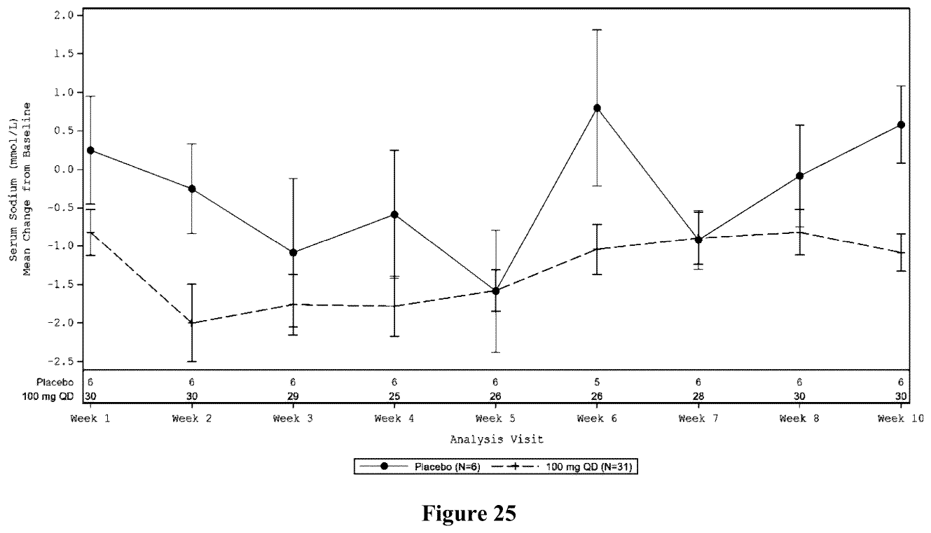

Serum sodium concentration decreased in the active treatment arms (FIG. 23, FIG. 24, and FIG. 25).

### DISCUSSION

This example describes a multicenter, prospective, randomized, placebo-controlled, double-blind, dose ranging study among adults with uncontrolled hypertension despite treatment with at least 2 background AHT medications. The results of this study demonstrate that aldosterone synthase inhibition with lorundrostat as an add-on therapy to a stable regimen of AHT medications was well tolerated, and induced a reduction in AOBP that was both clinically meaningful and statistically significant following treatment for 8 weeks.

Overall, 42 clinical trial sites across the USA participated in the trial, with 163 subjects randomized into Part 1 (of which 141 [86.5%] completed the trial on study drug) and 37 subjects randomized into Part 2 (of which 33 [89.2%] completed the trial on study drug). Baseline demographic, lifestyle and cardiovascular history characteristics were similar across all doses and study parts, with a relatively even distribution by sex, relevant race and ethnicity minority groups. As anticipated, the study population was primarily an older, overweight/obese group with a large minority of subjects being treated for Type II diabetes mellitus. Approximately 36% of participants were Black or African American. Furthermore, approximately half of the enrolled subjects had been prescribed a 2-drug AHT regimen, with most subjects (>77%) being treated with an ACEi or ARB, and a large majority (58%) being treated with a thiazide diuretic.

Analysis of the primary efficacy endpoint demonstrated that the majority of BP reductions achieved were within 4 weeks of starting therapy and were generally well-maintained for the remainder of the 8-week treatment period. Clinically meaningful placebo-adjusted reductions in AOBP SBP were seen across all doses tested, with statistically significant reductions of 9.58 mmHg (p=0.0114) and 7.81 mmHg (p=0.0422) seen in the 50 mg and 100 mg QD cohorts, respectively, in Part 1. Such reductions in SBP are salient to the effective treatment of subjects suffering with uncontrolled hypertension. In a recent meta-analysis of 147 randomized trials, 10 mmHg reductions in SBP were shown to reduce the risk of stroke by 41% and coronary heart disease by 22%. (Law, Morris, and Wald 2009) Furthermore, the reductions in AOBP SBP were validated by comparable reductions in 24-hour ABPM SBP, and confirmed with the associated benefit of lorundrostat reducing both central and nighttime ABPM SBP, two indicators of elevated cardiovascular event risk (Hermida et al. 2014; Mousa et al. 2004).

Beneficial effects were seen despite non-standardized background therapy, supporting the generalizability of effect across all subjects with uncontrolled hypertension. Moreover, the identification of sub-groups with statistically significant and clinically meaningful changes in AOBP SBP (i.e., subjects with a BMI ≥30 kg/m² and subjects with concomitant thiazide diuretic use) suggest the full efficacy of lorundrostat after 8 weeks of treatment may still be underestimated. Further studies exploring the effects of standardized background regimens and/or current prescribing paradigms with add-on lorundrostat therapy, or within specific sub-populations are warranted.

Suppressed renin is common among patients with hypertension and in theory, these patients should benefit most from drugs that decrease aldosterone production. Interestingly, subjects enrolled in Part 2 (i.e., subjects with non-suppressed renin levels) treated with lorundrostat demonstrated similar BP lowering efficacy to subjects with suppressed plasma renin levels (i.e., subjects enrolled in Part 1). While the PATHWAY-2 trial demonstrated other MRAs (such as spironolactone) are effective at lowering BP across a wide range of plasma renin levels, further study of lorundrostat in a population not selected for baseline PRA will be important to confirm the effect seen here.

Pharmacodynamic responses demonstrated dose-dependent reduction in serum aldosterone levels and a commensurate increase in plasma renin activity, in line with the mechanism of action of lorundrostat. No substantial decreases in serum cortisol levels were observed over the course of treatment and follow-up relative to placebo with percent change from baseline values of 20.6% to 51.4% (active) vs 37.7% (placebo) at Week 8, and −1.2 to 22.6% (active) vs 17.1% (placebo) at Week 12. A similar pattern was observed in Part 2, with active arm serum cortisol baseline levels of 11.1 μg/dL in the 100 mg QD dose group and no substantial changes observed over the course of treatment and subsequent follow-up. Most importantly, no adrenocortical insufficiency occurred during the trial.

Overall, lorundrostat was safe and well tolerated. Two (1.5%) subjects in Part 1 and one (3.2%) subject in Part 2 reported a treatment emergent SAE, of which the worsening of pre-existing hyponatremia was considered possibly related to study drug. No treatment emergent SAEs occurred in a subject treated with placebo. No deaths were reported in either Part 1 or Part 2.

At least one on-treatment TEAE was reported by 83 subjects in Part 1 (73 [54.9%] treated with lorundrostat versus 10 [33.3%] treated with placebo) and by 20 subjects in Part 2 (19 [61.3%] treated with lorundrostat versus 1 [16.7%] treated with placebo). Of the subjects treated with lorundrostat, 27 (20.3%) and 9 (29.0%) from Part 1 and Part 2, respectively, experienced TEAEs that were considered at least possibly related to study drug. The most commonly reported AE by PT was hyperkalemia and a decreased eGFR. All active doses saw modest increases in potassium levels at Week 8 relative to baseline ranging from 0.208 mmol/L (100 mg QD Part 2) to 0.341 mmol/L (25 mg BID Part 1). While seven subjects experienced transient elevated serum potassium greater than 6.0 mmol/L, none were considered an SAE and all rapidly resolved after discontinuation or dose adjustment, consistent with the short half-life of lorundrostat. It should be noted that one of these events was assessed as erroneous due to incorrect sample processing. In a manner similar to ACE inhibitors and ARBs, the BP lowering effect of lorundrostat led to a beneficial, reversible dose-dependent reduction in eGFR.

Hypotension related to treatment was seen in three subjects and was reversible with treatment cessation and expected based on lorundrostat's mechanism of action. Two of these subjects were randomized to the 100 mg QD cohorts (1 in both Part 1 and Part 2), and one to 12.5 mg BID. In addition, three subjects developed orthostatic hypotension, defined as a fall of 20 mmHg in SBP or 10 mmHg in DBP when going from sitting to standing. In each case, the episode resolved and did not recur for the remainder of the treatment period. Ongoing observation for symptoms of hypotension, even if minimal and reversible, is warranted for future clinical studies.

In conclusion, the primary objective of the study was to characterize the safety and efficacy of lorundrostat on blood pressure at 5 dosing levels and 2 dosing regimens versus placebo when administered orally for the treatment of uncontrolled hypertension as add-on therapy to stable background treatment. The results of this study indicate:
(a) Lorundrostat was effective at lowering BP in individuals with inadequately treated or treatment resistant hypertension;
(b) There was a dose- and exposure-response relationship, with mean placebo-adjusted reduction in SBP of 9.58 mmHg and 7.81 mmHg seen at doses of 50 mg QD and 100 mg QD, respectively;
(c) 24-hour ABPM, central BP and nighttime SBP values confirmed the reduction in SBP seen with AOBP, most prominently in the 100 mg QD dose cohort; and
(d) The safety dataset lorundrostat was safe and well tolerated, with no effects on serum cortisol and an expected modest increase in serum potassium with relatively few episodes of clinically meaningful hyperkalemia.

Future studies evaluating the long-term efficacy and safety of lorundrostat in patients with uncontrolled hypertension are warranted.

### BIBLIOGRAPHY

Ahmad Y, Francis D P, Bhatt D L, Howard J P. Renal denervation for hypertension: a systematic review and meta-analysis of randomized, blinded, placebo-controlled trials. Cardiovascular Interventions. 2021 Dec. 13; 14(23):2614-24.
Arriza J L, Weinberger C, Cerelli G, et al. Cloning of human mineralocorticoid receptor complementary DNA: structural and functional kinship with the glucocorticoid receptor. Science. 1987; 237(4812):268-275.
Baudrand R, Vaidya A. The low-renin hypertension phenotype: genetics and the role of the mineralocorticoid receptor. Int J Mol Sci. 2018;19(2):546.
Briet M, Schiffrin EL. Vascular actions of aldosterone. J Vasc Res. 2013; 50(2):89-99.
De Sousa K, Boulkroun S, Baron S, et al. Genetic, cellular, and molecular heterogeneity in adrenals with aldosterone-producing adenoma. Hypertension. 2020 April; 75(4):1034-44.
Decision Resources Group. Treatment algorithms: claims data analysis in hypertension. Published June 2019.
Dinh Cat A N, Friederich-Persson M, White A, Touyz R M. Adipocytes, aldosterone and obesity-related hypertension. Mol Endocrinol. 2016 July;57(1):F7-F21.
Faulkner J L, Belin de Chantemele E J. Leptin and aldosterone. Vitam Horm. 2019; 109:265-284.
Flynn C, Bakris G L. Interaction between adiponectin and aldosterone. Cardiorenal Medicine. 2011; 1(2):96-101.
Food and Drug Administration, 2020. Clinical drug interaction studies-cytochrome P450 enzyme-and transporter-mediated drug interactions guidance for industry. Center for Drug Evaluation and Research (CDER), US Department of Health and Human Services Food and Drug Administration, Silver Springs, MD.
Funder J W, Carey R M. Primary Aldosteronism: Where are we now? Where to from here? Hypertension. 2022 April; 79(4):726-735.
Good, D. W. (2007). Nongenomic actions of aldosterone on the renal tubule. Hypertension, 49(4), 728-739.
Grossmann, C., & Gekle, M. (2009). New aspects of rapid aldosterone signaling. Molecular and cellular endocrinology, 308(1-2), 53-62.
Hall J E, do Carmo J M, da Silva A A, Wang Z, Hall M E. Obesity-induced hypertension: interaction of neurohumoral and renal mechanisms. Circ Res. 2015 Mar. 13; 116(6):991-1006.
Hargovan M, Ferro A. Aldosterone synthase inhibitors in hypertension: current status and future possibilities. JRSM Cardiovasc Dis. 2014; 3:2048004014522440.
Hermida, R. C., D. E. Ayala, A. Mojon, M. H. Smolensky, F. Portaluppi, and J. R. Fernandez. 2014. ‘Sleep-time ambulatory blood pressure as a novel therapeutic target for cardiovascular risk reduction’, J Hum Hypertens, 28: 567-74.
Ishikawa K, Izawa Y, Ito H, et al. Aldosterone stimulates vascular smooth muscle cell proliferation via big mitogen-activated protein kinase 1 activation. Hypertension. 2005; 46(4):1046-1052.
Laffin L J, Kaufman H W, Chen Z, et al. Rise in blood pressure observed among US adults during the COVID-19 pandemic. Circulation. 2022 Jan. 18; 145(3):235-237.
Lainscak M, Pelliccia F, Rosano G, et al. Safety profile of mineralocorticoid receptor antagonists: Spironolactone and eplerenone. International journal of cardiology. 2015 Dec. 1; 200:25-9.
Law, M. R., J. K. Morris, and N. J. Wald. 2009. ‘Use of blood pressure lowering drugs in the prevention of cardiovascular disease: meta-analysis of 147 randomised trials in the context of expectations from prospective epidemiological studies’, BMJ, 338: b1665.
Mihailidou, A. S., & Funder, J. W. (2005). Nongenomic effects of mineralocorticoid receptor activation in the cardiovascular system. Steroids, 70(5-7), 347-351.
Million Hearts. Estimated hypertension prevalence, treatment, and control among U.S. adults. Updated Feb. 5, 2020. Accessed Feb. 5, 2021.
Mills J N, Thomas S, Williamson K S. The acute effect of hydrocortisone, deoxycorticosterone and aldosterone upon the excretion of sodium, potassium and acid by the human kidney. J Physiol. 1960; 151(2):312-331.
Mineralys Therapeutics, Trial on the Safety and Efficacy of Lorundrostat in Patients with Un-controlled Hypertension (Target-HTN). ClinicalTrials.gov identifier: NCT05001945. Available at: clinicaltrials.gov
Morgan ES, Tami Y, Hu K, et al. Antisense inhibition of angiotensinogen with IONIS-AGT-LRx: results of phase 1 and phase 2 studies. Basic to Translational Science. 2021 Jun. 1; 6(6):485-96.
Mulatero P, Monticone S, Deinum J, et al. Genetics, prevalence, screening and confirmation of primary aldosteronism: a position statement and consensus of the Working Group on Endocrine Hypertension of The European Society of Hypertension. Journal of hypertension. 2020 Oct. 1; 38(10):1919-28.
Muntner P, Hardy S T, Fine L J, et al. Trends in blood pressure control among US adults with hypertension, 1999-2000 to 2017-2018. JAMA. 2020 Sep. 22; 324(12):1190-1200
NCD Risk Factor Collaboration (NCDRF Collaboration). Worldwide trends in hypertension prevalence and progress in treatment and control from 1990 to 2019: a pooled analysis of 1201 population-representative studies with 104 million participants. Lancet. 2021 Sep. 11; 398(10304):957-980.
Nishiyama A, Yao L, Fan Y, et al. Involvement of aldosterone and mineralocorticoid receptors in rat mesangial cell proliferation and deformability. Hypertension. 2005; 45(4):710-716.
Oparil S, Schmieder R E. New approaches in the treatment of hypertension. Circ Res. 2015; 116(6):1074-1095.
Packer M. Leptin-aldosterone-neprilysin axis: identification of its distinctive role in the pathogenesis of the three phenotypes of heart failure in people with obesity. Circulation. 2018 Apr. 10; 137(15):1614-31.
Ramsay L E, Shelton J R, Tidd M J. The pharmacodynamics of single doses of prorenoate potassium and spironolactone in fludrocortisone treated normal subjects. Br J Clin Pharmacol. 1976; 3(3):475-482.
Romagni P, Rossi F, Guerrini L, Quirini C, Santiemma V. Aldosterone induces contraction of the resistance arteries in man. Atherosclerosis. 2003; 166(2):345-349.
Vaidya A, Mulatero P, Baudrand R, Adler G K. The expanding spectrum of primary aldosteronism: implications for diagnosis, pathogenesis, and treatment. Endocr Rev. 2018; 39(6):1057-1088.
Virani S S, Alonso A, Aparicio H J, et al; American Heart Association Council on Epidemiology and Prevention Statistics Committee and Stroke Statistics Subcommittee. Heart Disease and Stroke Statistics-2021 Update: A Report From the American Heart Association. Circulation. 2021 Feb. 23; 143(8):e254-e743.
Whelton P K, Carey R M, Aronow W S, et al. 2018 ACC/AHA/AAPA/ABC/ACPM/AGS/APhA/ASH/ASPC/NMA/PCNA Guideline for the prevention, detection, evaluation, and management of high blood pressure in adults: a report of the American College of Cardiology/American Heart Association Task Force on Clinical Practice Guidelines. Hypertension. 2018;71(6):e13-eli5.
Williams B. MacDonald T M, Morant S, et al. PATHWAY Study Group. Spironolactone versus placebo, bisoprolol, and doxazosin to determine the optimal treatment for drug-resistant hypertension (PATHWAY-2): a randomised, double-blind, crossover trial. Lancet. 2015; 386(10008):2059-2068.
Wright J T Jr, Williamson J D, Whelton P K, et al. SPRINT Research Group. A randomized trial of intensive versus standard blood-pressure control. N Engl J Med. 2015; 373(22):2103-2116.

What is claimed is:

1. A method of treating hypertension in a hypertensive subject, the method comprising administering to the subject, an amount of a diuretic and an amount of a CYP 11β2 beta hydroxylase inhibitor once or twice per day, wherein the amounts taken together are sufficient to treat hypertension in the hypertensive subject.

2. The method of claim 1, wherein the diuretic and CYP 11β2 beta hydroxylase inhibitor are administered in a combined pharmaceutical composition.

3. The method of claim 1, wherein the diuretic and CYP 11β2 beta hydroxylase inhibitor are administered concomitantly.

4. The method of any one of claims 1 to 3, wherein the amounts when taken together are effective to achieve a greater than additive therapeutic result in treating the subject.

5. The method of any one of claims 1 to 4, wherein the amount of CYP 11β2 beta hydroxylase inhibitor and the amount of diuretic when administered together is more effective to treat the subject than when each agent at the same amount is administered alone

6. A method of treating hypertension in a hypertensive subject who is taking at least one diuretic, the method comprising administering to the subject a CYP 11β2 beta hydroxylase inhibitor once or twice per day in an amount sufficient to thereby treat hypertension in the hypertensive subject.

7. The method of any one of claims 1 to 6, wherein the diuretic is a thiazide diuretic.

8. The method of any one of claims 1 to 7, wherein the hypertensive subject has a body mass index of at least 30, preferably more than 30, preferably 30-50, more preferably 30-40.

9. The method of any one of claims 1 to 8, wherein the hypertensive subject is a male hypertensive subject having a waist-hip ratio above 0.90, or a female hypertensive subject having a waist-hip ratio above 0.85.

10. The method of any one of claims 1 to 9, wherein the hypertensive subject has a serum leptin concentration of at least 30 ng/dL, preferably at least 35 ng/dL, more preferably at least 40 ng/dL, or more preferably 30-50 ng/dL, 30-45 ng/dL, or 35-50 ng/dL.

11. The method of any one of claims 1 to 10, wherein the hypertensive subject has:
a) a plasma aldosterone concentration of greater than or equal to 6 ng/dL by immunoassay in said subject; and/or
b) a plasma aldosterone concentration of greater than or equal to 1 ng/dL by LC-MS in said subject.

12. A method of treating hypertension in a hypertensive subject in need thereof, the method comprising
a) measuring
i) a systolic blood pressure of greater than 130 mmHg in said subject;
ii) a diastolic BP of greater than 80 mmHg in said subject; and
b) selecting a subject who is taking at least one diuretic; and
c) administering to said subject an effective amount of a CYP 11β2 beta hydroxylase inhibitor.

13. A method of treating hypertension in a hypertensive subject in need thereof, the method comprising
a) receiving an identification of the hypertensive subject as
i) having a systolic blood pressure of greater than 130 mmHg;
ii) having a diastolic BP of greater than 80 mmHg; and
iii) taking at least one diuretic; and
b) administering to said subject an effective amount of a CYP 11β2 beta hydroxylase inhibitor.

14. The method of claim 12 or 13, wherein the diuretic is a thiazide diuretic.

15. The method of any one of claims 12 to 13, wherein step a) further comprises measuring in said subject or receiving an identification of said subject having a body mass index (BMI) of at least 30, preferably more than 30, preferably 30-50, more preferably 30-40, or a waist-hip ratio above 0.90 if the hypertensive subject is male, or a waist-hip ratio above 0.85 if the hypertensive subject is female.

16. The method of any one of claims 12 to 15, wherein step a) further comprises measuring in said subject or receiving an identification of said subject having a serum leptin concentration of at least 30 ng/dL, preferably at least 35 ng/dL, more preferably at least 40 ng/dL, or more preferably 30-50 ng/dL, 30-45 ng/dL, or 35-50 ng/dL.

17. The method of any one of claims 12 to 16, wherein step a) further comprises:
a) measuring a plasma aldosterone concentration of greater than or equal to 6 ng/dL in said subject by immunoassay; or
b) measuring a plasma aldosterone concentration of greater than or equal to 1 ng/dL in said subject by LC-MS.

18. The method of any one of claims 1 to 16, wherein the hypertensive subject is taking or has taken a hypertension medication selected from an ACE inhibitor, an angiotensin receptor blocker, a calcium channel blocker, or a combination of two or more thereof.

19. The method of claim 18, wherein the hypertensive subject is taking or has taken at least two of said hypertension medications.

20. The method of any one of claims 1 to 19, wherein the CYP 11β2 beta hydroxylase inhibitor is administered to the subject once per day.

21. The method of any one of claims 1 to 20, wherein the CYP 11β2 beta hydroxylase inhibitor is administered in the morning.

22. The method of any one of claims 1 to 19, wherein the CYP 11β2 beta hydroxylase inhibitor is administered to the subject twice per day.

23. The method of any one of claims 1 to 22, wherein the CYP 11β2 beta hydroxylase inhibitor:
a) is administered daily for at least one week;
b) is administered daily for at least two weeks;
c) is administered daily for at least four weeks; or
d) is administered daily for at least eight weeks.

24. The method of any one of claims 1 to 23, wherein the hypertensive subject's ambulatory systolic blood pressure is reduced by at least 10 mmHg, by 10-55 mmHg, by 10-50 mmHg, by 10-45 mmHg, by 10-40 mmHg, by 10-35 mmHg, by 10-30 mmHg, by 10-25 mmHg, by 10-20 mmHg, or by 10-15 mmHg, relative to the hypertensive subject's ambulatory systolic blood pressure prior to administration of the CYP 11β2 beta hydroxylase inhibitor for a period of at least eight weeks.

25. The method of any one of claims 1 to 24, wherein the hypertensive subject's ambulatory diastolic blood pressure is reduced by at least 5 mmHg, by 5-25 mmHg, by 5-20 mmHg, or by 5-15 mmHg relative to the hypertensive subject's ambulatory diastolic blood pressure prior to administration of the CYP 11β2 beta hydroxylase inhibitor for a period of at least eight weeks.

26. The method of any one of claims 1 to 25, wherein the hypertensive subject's aldosterone level follows a substantially normal circadian rhythm.

27. The method of any one of claims 1 to 26, wherein the hypertensive subject's average systolic blood pressure during sleep is reduced (a) relative to the hypertensive subject's average systolic blood pressure during sleep prior to receiving the CYP 11β2 beta hydroxylase inhibitor, and/or (b) relative to the hypertensive subject's average daytime systolic blood pressure.

28. The method of any one of claims 1 to 27, wherein the hypertensive subject's average systolic blood pressure during sleep is reduced:
a) by at least 10%, by between 10% and 40%, by between 10% and 30%, or by between 10% and 20% relative to the hypertensive subject's average daytime systolic blood pressure; and/or
b) by at least 8 mmHg, by at least 10 mmHg, by between 8 and 55 mmHg, by between 10 and 45 mmHg, or by between 10 and 25 mmHg, relative to the hypertensive subject's average systolic blood pressure during sleep prior to receiving the CYP 11β2 beta hydroxylase inhibitor.

29. The method of any one of claims 1 to 28, wherein said CYP 11β2 beta hydroxylase inhibitor is selective for inhibition of CYP 11β2 beta hydroxylase activity relative to inhibition of CYP 1101 beta hydroxylase activity, preferably wherein the inhibition constant (Ki) for CYP 1101 beta hydroxylase divided by the Ki for CYP 11β2 beta hydroxylase is greater than 100.

30. The method of any one of claims 1 to 29, wherein said CYP 11β2 beta hydroxylase inhibitor is a compound of Formula (A) or a pharmaceutically acceptable salt thereof:

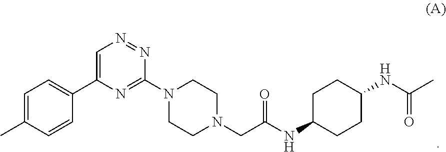 [SMILES: CC(=O)N[C@H]1CC[C@H](NC(=O)CN2CCN(c3nncc(-c4ccc(C)cc4)n3)CC2)CC1]

31. The method of claim 30, wherein the compound is in the form of an HBr salt of the compound of Formula (A).

32. The method of any one of claims 30 to 31, wherein:
a) between 5 mg and 50 mg of the CYP 11β2 beta hydroxylase inhibitor is administered orally twice a day, 12 hours apart;
b) between 10 mg and 30 mg of the CYP 11β2 beta hydroxylase inhibitor is administered orally twice a day, 12 hours apart;
c) between 30 mg and 120 mg of the CYP 11β2 beta hydroxylase inhibitor is administered orally once a day; or
d) between 40 mg and 110 mg of the CYP 11β2 beta hydroxylase inhibitor is administered orally once a day.

33. The method of any one of claims 30 to 31, wherein the diuretic is a thiazide diuretic, and wherein:
a) 12.5 mg of the CYP 11β2 beta hydroxylase inhibitor is administered orally once a day and the hypertensive subject experiences a placebo-adjusted reduction in systolic blood pressure of at least 5 mmHg, preferably by between 5 and 10 mmHg;
b) 50 mg of the CYP 11β2 beta hydroxylase inhibitor is administered orally once a day and the hypertensive subject experiences a placebo-adjusted reduction in systolic blood pressure of at least 10 mmHg, preferably by between 10 and 15 mmHg; or
c) 100 mg of the CYP 11β2 beta hydroxylase inhibitor is administered orally once a day and the hypertensive subject experiences a placebo-adjusted reduction in systolic blood pressure of at least 9 mmHg, preferably by between 9 and 15 mmHg.

34. The method of any one of claims 1 to 33, wherein the hypertensive subject does not have primary aldosteronism, preferably wherein the hypertensive subject has primary hypertension.

35. A method of identifying a subject for hypertension treatment with a CYP 11β2 beta hydroxylase inhibitor, the method comprising:
a) measuring:
i) a systolic blood pressure of greater than 130 mmHg in said subject; and
ii) a diastolic BP of greater than 80 mmHg in said subject; and
b) selecting a subject who is taking at least one diuretic;
thereby identifying said subject for hypertension treatment with a CYP 11β2 beta hydroxylase inhibitor.

36. The method of claim 35, wherein the diuretic is a thiazide diuretic.

37. The method of any one of claims 35 to 36, wherein step a) further comprises measuring a body mass index (BMI) of at least 30, preferably more than 30, preferably 30-50, more preferably 30-40, in said subject, or measuring a waist-hip ratio above 0.90 in said subject if the subject is male, or measuring a waist-hip ratio above 0.85 in said subject if the subject is female.

38. The method of any one of claims 35 to 37, wherein step a) further comprises measuring in said subject a serum leptin concentration of at least 30 ng/dL, preferably at least 35 ng/dL, more preferably at least 40 ng/dL, or more preferably 30-50 ng/dL, 30-45 ng/dL, or 35-50 ng/dL.

39. The method of any one of claims 35 to 38, wherein step a) further comprises:
a) measuring a plasma aldosterone concentration of greater than or equal to 6 ng/dL by immunoassay in said subject; or
b) measuring a plasma aldosterone concentration of greater than or equal to 1 ng/dL by LC-MS in said subject.

40. A package comprising:
a) a first pharmaceutical composition comprising an amount of a compound of Formula (A) or a pharmaceutically acceptable salt thereof: 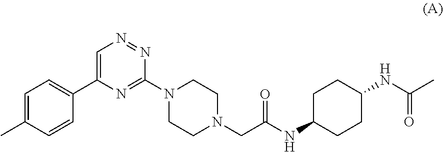 [SMILES: CC(=O)N[C@H]1CC[C@H](NC(=O)CN2CCN(c3nncc(-c4ccc(C)cc4)n3)CC2)CC1]
and a pharmaceutically acceptable carrier;
b) a second pharmaceutical composition comprising an amount of a thiazide diuretic and a pharmaceutically acceptable carrier; and
c) instructions for use of the first and second pharmaceutical compositions together to treat a subject afflicted with hypertension.

41. A compound of Formula (A) or a pharmaceutically acceptable salt thereof: 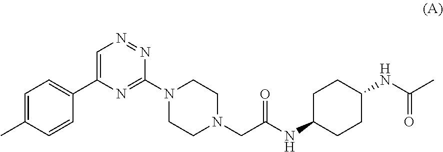 [SMILES: CC(=O)N[C@H]1CC[C@H](NC(=O)CN2CCN(c3nncc(-c4ccc(C)cc4)n3)CC2)CC1]
for use as an add-on therapy or in combination with a thiazide diuretic in treating a subject afflicted with hypertension.

42. A pharmaceutical composition comprising
a) an amount of the compound of Formula (A) or a pharmaceutically acceptable salt thereof: 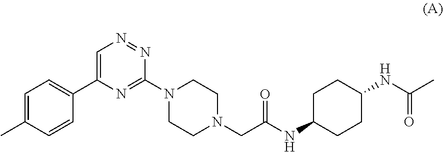 [SMILES: CC(=O)N[C@H]1CC[C@H](NC(=O)CN2CCN(c3nncc(-c4ccc(C)cc4)n3)CC2)CC1]
b) and an amount of a thiazide diuretic for use in treating a subject afflicted with hypertension, wherein the compound of Formula (A) and the thiazide diuretic are administered simultaneously, contemporaneously or concomitantly.

43. A pharmaceutical composition comprising
a) an amount of the compound of Formula (A) or a pharmaceutically acceptable salt thereof: 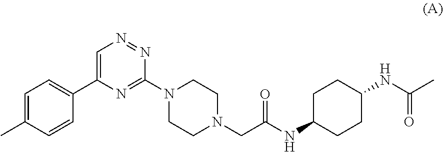 [SMILES: CC(=O)N[C@H]1CC[C@H](NC(=O)CN2CCN(c3nncc(-c4ccc(C)cc4)n3)CC2)CC1]
b) and an amount of a thiazide diuretic
wherein the compound of Formula (A) and thiazide diuretic are each present in amounts which together are effective to treat hypertension in a hypertensive subject.

44. A pharmaceutical composition comprising an amount of the compound of Formula (A) or a pharmaceutically acceptable salt thereof: 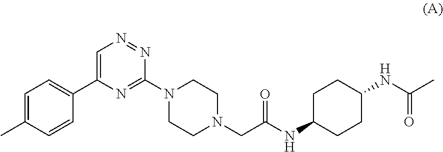 [SMILES: CC(=O)N[C@H]1CC[C@H](NC(=O)CN2CCN(c3nncc(-c4ccc(C)cc4)n3)CC2)CC1]
for use in treating a subject afflicted with hypertension as an add-on therapy or in combination with a thiazide diuretic.

45. A therapeutic package for dispensing to, or for use in dispensing to, a subject afflicted with hypertension, which comprises:
a) one or more unit doses, each such unit dose comprising:
i) an amount of the compound of Formula (A) or a pharmaceutically acceptable salt thereof: 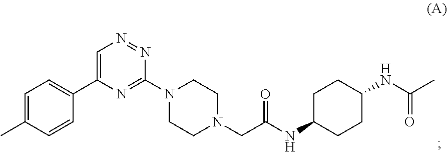 [SMILES: CC(=O)N[C@H]1CC[C@H](NC(=O)CN2CCN(c3nncc(-c4ccc(C)cc4)n3)CC2)CC1]
and
ii) an amount of a thiazide diuretic,
wherein the respective amounts of said compound of Formula (A) and said thiazide diuretic in said unit dose are effective, upon concomitant administration to said subject, to treat the subject, and
b) a finished pharmaceutical container therefor, said container containing said unit dose or unit doses, said container further containing or comprising labeling directing the use of said package in the treatment of said subject.

46. A pharmaceutical composition in unit dosage form, useful in treating a subject afflicted with hypertension, which comprises:
a) an amount of the compound of Formula (A) or a pharmaceutically acceptable salt thereof: 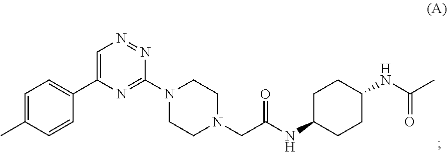 [SMILES: CC(=O)N[C@H]1CC[C@H](NC(=O)CN2CCN(c3nncc(-c4ccc(C)cc4)n3)CC2)CC1]
and
b) an amount of a thiazide diuretic,
wherein the respective amounts of said compound of Formula (A) and said thiazide diuretic in said composition are effective, upon concomitant administration to said subject of one or more of said unit dosage forms of said composition, to treat the subject.
